SEQUENCE LISTING (87 sequences)

SEQ ID NO: 1 - protein, 770 aa, Artificial Sequence
  Synthetic Polypeptide
MLPGLALLLLAAWTARALEVPTDGNAGLLAEPQIAMFCGRLNMHMNVQNGKWDSDPSGTKTCIDTKEGILQYCQEVYPELQITNVVEANQPVTIQNWCKRGRKQCKTHPHFVIPYRCLVGEFVSDALLVPDKCKFLHQERMDVCETHLHWHTVAKETCSEKSTNLHDYGMLLPCGIDKFRGVEFVCCPLAEESDNVDSADAEEDDSDVWWGGADTDYADGSEDKVVEVAEEEEVAEVEEEEADDDEDDEDGDEVEEEAEEPYEEATERTTSIATTTTTTTESVEEVVREVCSEQAETGPCRAMISRWYFDVTEGKCAPFFYGGCGGNRNNFDTEEYCMAVCGSAMSQSLLKTTQEPLARDPVKLPTTAASTPDAVDKYLETPGDENEHAHFQKAKERLEAKHRERMSQVMREWEEAERQAKNLPKADKKAVIQHFQEKVESLEQEAANERQQLVETHMARVEAMLNDRRRLALENYITALQAVPPRPRHVFNMLKKYVRAEQKDRQHTLKHFEHVRMVDPKKAAQIRSQVMTHLRVIYERMNQSLSLLYNVPAVAEEIQDEVDELLQKEQNYSDDVLANMISEPRISYGNDALMPSLTETKTTVELLPVNGEFSLDDLQPWHSFGADSVPANTENEVEPVDARPAADRGLTTRPGSGLTNIKTEEISEVKMDAEFRHDSGYEVHHQKLVFFAEDVGSNKGAIIGLMVGGVVIATVIVITLVMLKKKQYTSIHHGVVEVDAAVTPEERHLSKMQQNGYENPTYKFFEQMQN

SEQ ID NO: 2 - protein, 42 aa, Artificial Sequence [sequence omitted]

SEQ ID NO: 3 - protein, 42 aa, Artificial Sequence
  Synthetic Polypeptide
DAEFRHDSGYEVHHQKLVDFAEDVGSNKGAIIGPMVGGVVIA

SEQ ID NO: 4 - protein, 42 aa, Artificial Sequence
  Synthetic Polypeptide
DAEFRHDSGYEVHHQKLVFPAEDVGSNKGAIIGLMVGGVVIA

SEQ ID NO: 5 - protein, 41 aa, Artificial Sequence
  Synthetic Polypeptide
DAEFRHDSGYEVHHQKLVDFAEDVGSNKGAIIGLMVGGVVI

SEQ ID NO: 6 - protein, 42 aa, Artificial Sequence
  Synthetic Polypeptide
DAEFRHDSGYEVHHQKLVPFAEDVGSNKGAIIGLMVGGVVIA

SEQ ID NO: 7 - protein, 42 aa, Artificial Sequence
  Synthetic Polypeptide
DAEFRHDSGYEVHHQKLPFFAEDVGSNKGAIIGLMVGGVVIA

SEQ ID NO: 8 - protein, 54 aa, Artificial Sequence
  Synthetic Polypeptide
DAEFRHDSGYEVHHQKLVPFAEDVGSNKGAIIGLMVGGVVIATVIVITLVMLKK

SEQ ID NO: 9 - protein, 23 aa, Artificial Sequence
  Synthetic Polypeptide
AIIGLMVGGVVIATVIVITLVML

SEQ ID NO: 10 - protein, 7 aa, Artificial Sequence
  Synthetic Polypeptide
KLVDFAE

SEQ ID NO: 11 - protein, 7 aa, Artificial Sequence
  Synthetic Polypeptide
KLVFPAE

SEQ ID NO: 12 - protein, 7 aa, Artificial Sequence
  Synthetic Polypeptide
KLVPFAE

SEQ ID NO: 13 - protein, 54 aa, Artificial Sequence
  Synthetic Polypeptide
DAEFRHDSGYEVHHQKLVFFAEDVGSNKGAIIGLMVGGVVIATVIVITLVMLKK

SEQ ID NO: 14 - protein, 55 aa, Artificial Sequence
  Synthetic Polypeptide
DAEFRHDSGYEVHHQKLVFFAEDVGSNKGAIIGLMVGGVVIATVIVITLVMLKKK

SEQ ID NO: 15 - protein, 55 aa, Artificial Sequence
  Synthetic Polypeptide
DAEFRHDSGYEVHHQKLVFFAEDVGSNKGAIIGLMVGGVVIATVIVITLVMAKKK

SEQ ID NO: 16 - protein, 55 aa, Artificial Sequence
  Synthetic Polypeptide
DAEFRHDSGYEVHHQKLVFFAEDVGSNKGAIIGLMVGGVVIATVIVITLVMLRKK

SEQ ID NO: 17 - protein, 55 aa, Artificial Sequence
  Synthetic Polypeptide
DAEFRHDSGYEVHHQKLVFFAEDVGSNKGAIIGLMVGGVVIATVIVITLVMARKK

SEQ ID NO: 18 - protein, 23 aa, Artificial Sequence
  Synthetic Polypeptide
AIIGLMVGGVVIATVIVITLVMA

SEQ ID NO: 19 - protein, 17 aa, Artificial Sequence
  Synthetic Polypeptide
MGVKVLFALICIAVAEA

SEQ ID NO: 20 - DNA, 51 bp, Artificial Sequence
  Synthetic Polynucleotide
atgggcgtgaaggtcctgttcgccctgatttgcatcgccgtcgcagaggca

SEQ ID NO: 21 - protein, 19 aa, Artificial Sequence
  Synthetic Polypeptide
MGWSCIILFLVATATGVHS

SEQ ID NO: 22 - DNA, 139 bp, Artificial Sequence
  Synthetic Polynucleotide
atgggatggagctgtatcatcctcttcttggtagcaacagctacaggtaaggggctcacagtagcaggcttgaggtctggacatatatatgggtgacaatgacatccactttgcctttctctccacaggtgtccactcc

SEQ ID NO: 23 - protein, 10 aa, Artificial Sequence
  Synthetic Polypeptide
GRKKRRQRRR

SEQ ID NO: 24 - protein, 11 aa, Artificial Sequence
  Synthetic Polypeptide
YGRKKRRQRRR

SEQ ID NO: 25 - protein, 12 aa, Artificial Sequence
  Synthetic Polypeptide
GRKKRRQRRRPQ

SEQ ID NO: 26 - protein, 20 aa, Artificial Sequence
  Synthetic Polypeptide
RRRRRRRRRRRRRRRRRRRR

SEQ ID NO: 27 - protein, 18 aa, Artificial Sequence
  Synthetic Polypeptide
KLALKLALKALKAALKLA

SEQ ID NO: 28 - protein, 16 aa, Artificial Sequence
  Synthetic Polypeptide
AAVALLPAVLLALLAP

SEQ ID NO: 29 - protein, 27 aa, Artificial Sequence
  Synthetic Polypeptide
GLAFLGFLGAAGSTMGAWSQPKKKRKV

SEQ ID NO: 30 - protein, 16 aa, Artificial Sequence
  Synthetic Polypeptide
RQIKIWFQNRRMKWKK

SEQ ID NO: 31 - protein, 20 aa, Artificial Sequence
  Synthetic Polypeptide
KETWWETWWTEWSQPKKRKV

SEQ ID NO: 32 - protein, 27 aa, Artificial Sequence
  Synthetic Polypeptide
GWTLNSAGYLLGKINLKALAALAKKIL

SEQ ID NO: 33 - protein, 34 aa, Artificial Sequence
  Synthetic Polypeptide
DAATATRGRSAASRPTQRPRAPARSASRPRRPVQ

SEQ ID NO: 34 - RNA, 11 nt, Artificial Sequence
  Synthetic Polynucleotide
gccaccauggg

SEQ ID NO: 35 - protein, 55 aa, Artificial Sequence
  Synthetic Polypeptide
DAEFRHDSGYEVHHQKLVPFAEDVGSNKGAIIGLMVGGVVIATVIVITLVMLKKK

SEQ ID NO: 36 - protein, 55 aa, Artificial Sequence
  Synthetic Polypeptide
DAEFRHDSGYEVHHQKLVPFAEDVGSNKGAIIGLMVGGVVIATVIVITLVMAKKK

SEQ ID NO: 37 - protein, 55 aa, Artificial Sequence
  Synthetic Polypeptide
DAEFRHDSGYEVHHQKLVPFAEDVGSNKGAIIGLMVGGVVIATVIVITLVMLRKK

SEQ ID NO: 38 - protein, 55 aa, Artificial Sequence
  Synthetic Polypeptide
DAEFRHDSGYEVHHQKLVPFAEDVGSNKGAIIGLMVGGVVIATVIVITLVMARKK

SEQ ID NO: 39 - protein, 7 aa, Artificial Sequence
  Synthetic Polypeptide
KLVFFAE

SEQ ID NO: 40 - protein, 59 aa, Artificial Sequence
  Synthetic Polynucleotide
ATGGGCGTGAAGGTCCTGTTCGCCCTGATTTGCATCGCCGTCGCAGAGGCAGATGCAGA

SEQ ID NO: 41 - protein, 59 aa, Artificial Sequence
  Synthetic Polynucleotide
TCTGCATCTGCCTCTGCGACGGCGATGCAAATCAGGGCGAACAGGACCTTCACGCCCAT

SEQ ID NO: 42 - protein, 33 aa, Artificial Sequence
  Synthetic Polynucleotide
GTCGCAGAGGCAGATGCAGAATTCCGACATGAC

SEQ ID NO: 43 - protein, 33 aa, Artificial Sequence
  Synthetic Polynucleotide
GCGCGGATATCCTAGTTCTGCATCTGCTCAAAG

SEQ ID NO: 44 - protein, 37 aa, Artificial Sequence
  Synthetic Polynucleotide
GCGCGAAGCTTGCCACCATGGGCGTGAAGGTCCTGTT

SEQ ID NO: 45 - protein, 33 aa, Artificial Sequence; identical to SEQ ID NO: 43
  Synthetic Polynucleotide

SEQ ID NO: 46 - protein, 37 aa, Artificial Sequence; identical to SEQ ID NO: 44
  Synthetic Polynucleotide

SEQ ID NO: 47 - protein, 39 aa, Artificial Sequence
  Synthetic Polynucleotide
GCGCGGATATCTTACTACTTCTTCAGCATCACCAAGGTG

SEQ ID NO: 48 - protein, 37 aa, Artificial Sequence
  Synthetic Polynucleotide
GCGCGGATATCTTACTACAGCATCACCAAGGTGATGA

SEQ ID NO: 49 - protein, 37 aa, Artificial Sequence
  Synthetic Polynucleotide
GCGCGGATATCTTACTACACCAAGGTGATGACGATCA

SEQ ID NO: 50 - protein, 37 aa, Artificial Sequence
  Synthetic Polynucleotide
GCGCGGATATCTTACTAGGTGATGACGATCACTGTCG

SEQ ID NO: 51 - protein, 37 aa, Artificial Sequence
  Synthetic Polynucleotide
GCGCGGATATCTTACTAGACGATCACTGTCGCTATGA

SEQ ID NO: 52 - protein, 37 aa, Artificial Sequence
  Synthetic Polynucleotide
GCGCGGATATCTTACTACGCTATGACAACACCGCCCA

SEQ ID NO: 53 - protein, 37 aa, Artificial Sequence; identical to SEQ ID NO: 44
  Synthetic Polynucleotide

SEQ ID NO: 54 - protein, 59 aa, Artificial Sequence
  Synthetic Polynucleotide
ACCATGGGTCCAATGATTGCACCTTTGTTTGAACCCACATCTTCTGCAAAGTCCACCAA

SEQ ID NO: 55 - protein, 59 aa, Artificial Sequence
  Synthetic Polynucleotide
TTGGTGGACTTTGCAGAAGATGTGGGTTCAAACAAAGGTGCAATCATTGGACCCATGGT

SEQ ID NO: 56 - protein, 39 aa, Artificial Sequence; identical to SEQ ID NO: 47
  Synthetic Polynucleotide

SEQ ID NO: 57 - protein, 37 aa, Artificial Sequence; identical to SEQ ID NO: 44
  Synthetic Polynucleotide

SEQ ID NO: 58 - protein, 39 aa, Artificial Sequence; identical to SEQ ID NO: 47
  Synthetic Polynucleotide

SEQ ID NO: 59 - protein, 37 aa, Artificial Sequence; identical to SEQ ID NO: 44
  Synthetic Polynucleotide

SEQ ID NO: 60 - protein, 26 aa, Artificial Sequence
  Synthetic Polynucleotide
TCTTCTGCAGGGAACACCAATTTTTG

SEQ ID NO: 61 - protein, 26 aa, Artificial Sequence
  Synthetic Polynucleotide
CAAAAATTGGTGTTCCCTGCAGAAGA

SEQ ID NO: 62 - protein, 39 aa, Artificial Sequence; identical to SEQ ID NO: 47
  Synthetic Polynucleotide

SEQ ID NO: 63 - protein, 37 aa, Artificial Sequence; identical to SEQ ID NO: 44
  Synthetic Polynucleotide

SEQ ID NO: 64 - protein, 39 aa, Artificial Sequence; identical to SEQ ID NO: 47
  Synthetic Polynucleotide

SEQ ID NO: 65 - protein, 7 aa, Artificial Sequence
  Synthetic Polypeptide
KLPFFAE

SEQ ID NO: 66 - protein, 54 aa, Artificial Sequence
  Synthetic Polypeptide
DAEFRHDSGYEVHHQKLVDFAEDVGSNKGAIIGPMVGGVVIATVIVITLVMLKK

SEQ ID NO: 67 - protein, 55 aa, Artificial Sequence
  Synthetic Polypeptide
DAEFRHDSGYEVHHQKLVDFAEDVGSNKGAIIGPMVGGVVIATVIVITLVMLKKK

SEQ ID NO: 68 - protein, 55 aa, Artificial Sequence
  Synthetic Polypeptide
DAEFRHDSGYEVHHQKLVDFAEDVGSNKGAIIGPMVGGVVIATVIVITLVMAKKK

SEQ ID NO: 69 - protein, 55 aa, Artificial Sequence
  Synthetic Polypeptide
DAEFRHDSGYEVHHQKLVDFAEDVGSNKGAIIGPMVGGVVIATVIVITLVMLRKK

SEQ ID NO: 70 - protein, 55 aa, Artificial Sequence
  Synthetic Polypeptide
DAEFRHDSGYEVHHQKLVDFAEDVGSNKGAIIGPMVGGVVIATVIVITLVMARKK

SEQ ID NO: 71 - protein, 54 aa, Artificial Sequence
  Synthetic Polypeptide
DAEFRHDSGYEVHHQKLVFPAEDVGSNKGAIIGLMVGGVVIATVIVITLVMLKK

SEQ ID NO: 72 - protein, 55 aa, Artificial Sequence
  Synthetic Polypeptide
DAEFRHDSGYEVHHQKLVFPAEDVGSNKGAIIGLMVGGVVIATVIVITLVMLKKK

SEQ ID NO: 73 - protein, 55 aa, Artificial Sequence
  Synthetic Polypeptide
DAEFRHDSGYEVHHQKLVFPAEDVGSNKGAIIGLMVGGVVIATVIVITLVMAKKK

SEQ ID NO: 74 - protein, 55 aa, Artificial Sequence
  Synthetic Polypeptide
DAEFRHDSGYEVHHQKLVFPAEDVGSNKGAIIGLMVGGVVIATVIVITLVMLRKK

SEQ ID NO: 75 - protein, 55 aa, Artificial Sequence
  Synthetic Polypeptide
DAEFRHDSGYEVHHQKLVFPAEDVGSNKGAIIGLMVGGVVIATVIVITLVMARKK

SEQ ID NO: 76 - protein, 54 aa, Artificial Sequence
  Synthetic Polypeptide
DAEFRHDSGYEVHHQKLVDFAEDVGSNKGAIIGLMVGGVVIATVIVITLVMLKK

SEQ ID NO: 77 - protein, 55 aa, Artificial Sequence
  Synthetic Polypeptide
DAEFRHDSGYEVHHQKLVDFAEDVGSNKGAIIGLMVGGVVIATVIVITLVMLKKK

SEQ ID NO: 78 - protein, 55 aa, Artificial Sequence
  Synthetic Polypeptide
DAEFRHDSGYEVHHQKLVDFAEDVGSNKGAIIGLMVGGVVIATVIVITLVMAKKK

SEQ ID NO: 79 - protein, 55 aa, Artificial Sequence
  Synthetic Polypeptide
DAEFRHDSGYEVHHQKLVDFAEDVGSNKGAIIGLMVGGVVIATVIVITLVMLRKK

SEQ ID NO: 80 - protein, 55 aa, Artificial Sequence
  Synthetic Polypeptide
DAEFRHDSGYEVHHQKLVDFAEDVGSNKGAIIGLMVGGVVIATVIVITLVMARKK

SEQ ID NO: 81 - protein, 54 aa, Artificial Sequence
  Synthetic Polypeptide
DAEFRHDSGYEVHHQKLPFFAEDVGSNKGAIIGLMVGGVVIATVIVITLVMLKK

SEQ ID NO: 82 - protein, 55 aa, Artificial Sequence
  Synthetic Polypeptide
DAEFRHDSGYEVHHQKLPFFAEDVGSNKGAIIGLMVGGVVIATVIVITLVMLKKK

SEQ ID NO: 83 - protein, 55 aa, Artificial Sequence
  Synthetic Polypeptide
DAEFRHDSGYEVHHQKLPFFAEDVGSNKGAIIGLMVGGVVIATVIVITLVMAKKK

SEQ ID NO: 84 - protein, 55 aa, Artificial Sequence
  Synthetic Polypeptide
DAEFRHDSGYEVHHQKLPFFAEDVGSNKGAIIGLMVGGVVIATVIVITLVMLRKK

SEQ ID NO: 85 - protein, 55 aa, Artificial Sequence
  Synthetic Polypeptide
DAEFRHDSGYEVHHQKLPFFAEDVGSNKGAIIGLMVGGVVIATVIVITLVMARKK

SEQ ID NO: 86 - protein, 23 aa, Artificial Sequence
  Synthetic Polypeptide
AIIGPMVGGVVIATVIVITLVML

SEQ ID NO: 87 - protein, 23 aa, Artificial Sequence
  Synthetic Polypeptide
AIIGPMVGGVVIATVIVITLVMA

(19) United States
(12) Patent Application Publication
(10) Pub. No.: US 2024/0050524 A1
(43) Pub. Date: Feb. 15, 2024

(54) DELIVERY OF ABETA VARIANTS FOR AGGREGATION INHIBITION

(71) Applicant: Baylor College of Medicine, Houston, TX (US)

(72) Inventors: Kyung-Won Park, Houston, TX (US); Joanna Jankowsky, Houston, TX (US)

(21) Appl. No.: 18/267,980

(22) PCT Filed: Dec. 15, 2021
(86) PCT No.: PCT/US2021/072944

§ 371 Date: Jun. 16, 2023

Related U.S. Application Data
(60) Provisional application No. 63/127,815, filed on Dec. 18, 2020.

(51) Int. Cl. A61K 38/17; A61K 48/00; A61P 25/28

(52) U.S. Cl. CPC A61K 38/1716; A61K 48/0041; A61P 25/28

(57) ABSTRACT

Aspects of the present disclosure are directed to compositions and methods for treating a subject having a neurodegenerative disorder, disease, or condition. Certain aspects relate to treatment with a therapeutically effective amount of a composition comprising a vector encoding an Aβ peptide variant. Further aspects relate to methods of inhibiting aggregation of endogenous Aβ peptide in vivo by contacting at least one such peptide with a therapeutically effective amount of an expressed Aβ peptide variant from a vector encoding the Aβ peptide variant, said vector in a composition.

### CROSS-REFERENCE BETWEEN RELATED APPLICATIONS

This application claims priority to U.S. Provisional Patent Application Ser. No. 63/127,815 filed Dec. 18, 2020, which is incorporated by reference herein in its entirety.

This invention was made with government support under NS092615, AG054160, AG056028, and AG058188, awarded by the National Institutes of Health. The government has certain rights in the invention.

### BACKGROUND

### I. Technical Field

Aspects of this disclosure relates to at least the fields of cell biology, molecular biology, protein biology, neurobiology, and medicine.

### II. Background

Alzheimer's disease (AD) is a devastating neurodegenerative disorder and the most common cause of dementia. The pathological hallmarks of AD are the presence of neurofibrillary tangles and amyloid deposits in the patient's brain. Amyloid beta (Aβ) peptides and their aggregated forms are the major component of the amyloid plaques. Aβ1-42 especially is particularly susceptible to aggregation, forming oligomers, protofibrils, insoluble fibrils, and plaques. Engineered antibody therapies have been effective at lowering Aβ aggregation; however, the side effect profile and need for repeated intravenous delivery present obstacles to widespread use of engineered antibodies for treatment of neurodegenerative disorders like AD. Thus, there is a need in the art for novel treatment approaches having high specificity, low toxicity, and an extended half-life in vivo which are capable of preventing the oligomerization and aggregation of Aβ peptides.

### SUMMARY

Aspects of the present disclosure address needs in the art by providing methods and compositions for treating subjects with neurodegenerative diseases, disorders, or conditions (e.g., Alzheimer's disease). Accordingly, provided herein, in some aspects, are methods and compositions for treating a subject with a neurodegenerative disease, disorder, or condition and/or for inhibiting aggregation of, or promoting disaggregation of, amyloid beta (Aβ) peptide in vivo, comprising administration of a therapeutically effective amount of a composition comprising a vector encoding an Aβ peptide variant, or a fragment or functional derivative thereof. In some embodiments, the disclosed methods further comprise diagnosing the subject with the neurodegenerative disease, disorder, or condition; diagnosing the subject as having symptoms of the neurodegenerative disease, disorder, or condition; or diagnosing the subject as being at risk of having the neurodegenerative disease, disorder, or condition. In some embodiments, the neurodegenerative disease, disorder, or condition is Alzheimer's disease, Parkinson's disease, Parkinson's disease dementia, vascular dementia, cerebral amyloid angiopathy, dementia with Lewy bodies, chronic traumatic encephalopathy, Down syndrome, and/or pathological aging. In some embodiments, the neurodegenerative disease, disorder, or condition is Alzheimer's disease.

Embodiments of the disclosure include methods and compositions for treating a subject having a neurodegenerative disease, disorder, or condition and/or for inhibiting aggregation of Aβ peptide in vivo. Methods of the disclosure can include 1, 2, 3, 4, 5, or more of the following steps: providing an Aβ peptide variant to a subject; providing one or more additional therapies for the neurodegenerative disease, disorder, or condition to a subject; diagnosing the subject with the neurodegenerative disease, disorder, or condition; diagnosing the subject as having symptoms of the neurodegenerative disease, disorder, or condition; and diagnosing the subject as being at risk of having the neurodegenerative disease, disorder, or condition. Certain embodiments of the disclosure may exclude one or more of the preceding elements and/or steps.

Disclosed herein, in some aspects, is a method of treating or preventing a neurodegenerative disease, disorder, or condition in a subject, comprising administering to the subject a therapeutically effective amount of a composition comprising a vector encoding an Aβ peptide variant, or a fragment or functional derivative thereof. In some embodiments, administering the vector encoding an Aβ peptide variant or a fragment or functional derivative thereof prevents or decreases protein misfolding, endogenous Aβ peptide aggregation, amyloid plaque formation, neuroinflammation, neurodegeneration, neuronal loss, or synaptic loss; decreases tau levels, phosphorylation of tau, or phosphorylated tau levels; slows seeding of tau or seeding of endogenous Aβ peptide; and/or promotes cognitive improvement. In some embodiments, administering the vector encoding an Aβ peptide variant prevents or decreases formation of endogenous Aβ peptide oligomers, protofibrils, fibrils, or plaques. In some embodiments, administering the vector encoding an Aβ peptide variant prevents or decreases cytotoxicity of endogenous Aβ peptide aggregate.

Disclosed herein, in some aspects, is a method of inhibiting aggregation of endogenous Aβ peptide in vivo, comprising contacting at least one such peptide with a therapeutically effective amount of an expressed Aβ peptide variant from a vector encoding the Aβ peptide variant, said vector in a composition. In some embodiments, inhibiting aggregation of endogenous Aβ peptide treats or prevents a neurodegenerative disease, disorder, or condition in a subject. In some embodiments, inhibiting aggregation of endogenous Aβ peptide prevents or decreases formation of endogenous Aβ peptide oligomers, protofibrils, fibrils, or plaques. In some embodiments, inhibiting aggregation of endogenous Aβ peptide prevents or decreases cytotoxicity of endogenous Aβ peptide aggregate.

In some embodiments, the method further comprises diagnosing the subject with the neurodegenerative disease, disorder, or condition; diagnosing the subject as having symptoms of the neurodegenerative disease, disorder, or condition; and diagnosing the subject as being at risk of having the neurodegenerative disease, disorder, or condition. In some embodiments, the neurodegenerative disease, disorder, or condition is Alzheimer's disease, Parkinson's disease, Parkinson's disease dementia, vascular dementia, cerebral amyloid angiopathy, dementia with Lewy bodies, chronic traumatic encephalopathy, Down syndrome, and/or pathological aging. In some embodiments, the neurodegenerative disease, disorder, or condition is Alzheimer's disease.

Disclosed herein, in some aspects, is a pharmaceutical composition comprising a vector encoding an Aβ peptide variant.

In some embodiments of the methods and compositions disclosed herein, the Aβ peptide variant comprises an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity with SEQ ID NO:3, SEQ ID NO:4, SEQ ID NO:5, or SEQ ID NO:6, or a fragment or functional derivative thereof. In some embodiments, the Aβ peptide variant comprises an amino acid sequence having at least 80% identity with SEQ ID NO:3, SEQ ID NO:4, SEQ ID NO:5, or SEQ ID NO:6, or a fragment or functional derivative thereof. In some embodiments, the Aβ peptide variant comprises SEQ ID NO:3, SEQ ID NO:4, SEQ ID NO:5, or SEQ ID NO:6, or a fragment or functional derivative thereof.

In some embodiments, the Aβ peptide variant comprises an amino acid sequence having at least 85, 90, 91, 92, 93, 94, 95, 96, 97, 98, or 99% identity with SEQ ID NO:3, or a fragment or functional derivative thereof. In some embodiments, the Aβ peptide variant comprises SEQ ID NO:3, or a fragment or functional derivative thereof. In some embodiments, the Aβ peptide variant having at least 85, 90, 91, 92, 93, 94, 95, 96, 97, 98, or 99% identity with or comprising SEQ ID NO:3 comprises a N-terminal truncation. In some embodiments, the N-terminal truncation comprises a 1, 2, 3, 4, 5, 6, 7, 8, 9, 10, 11, 12, 13, 14, 15, 16, 17, 18, 19, 20, 21, or 22 amino acid truncation. In some embodiments, the Aβ peptide variant having at least 85, 90, 91, 92, 93, 94, 95, 96, 97, 98, or 99% identity with or comprising SEQ ID NO:3 comprises a C-terminal truncation. In some embodiments, the C-terminal truncation comprises a 1, 2, 3, 4, 5, 6, 7, 8, 9, 10, 11, 12, 13, 14, 15, 16, 17, 18, 19, 20, 21, 22, 23, 24, 25, 26, 27, or 28 amino acid truncation. In some embodiments, the fragment or functional derivative of SEQ ID NO:3 comprises an amino acid sequence having at least 85, 86, 87, 88, 89, 90, 91, 92, 93, 94, 95, 96, 97, 98, or 99 sequence identity with KLVDFAE (SEQ ID NO:10).

In some embodiments, the Aβ peptide variant comprises an amino acid sequence having at least 85, 90, 91, 92, 93, 94, 95, 96, 97, 98, or 99% identity with SEQ ID NO:4, or a fragment or functional derivative thereof. In some embodiments, the Aβ peptide variant comprises SEQ ID NO:4, or a fragment or functional derivative thereof. In some embodiments, the Aβ peptide variant having at least 85, 90, 91, 92, 93, 94, 95, 96, 97, 98, or 99% identity with or comprising SEQ ID NO:4 comprises a N-terminal truncation. In some embodiments, the N-terminal truncation comprises a 1, 2, 3, 4, 5, 6, 7, 8, 9, 10, 11, 12, 13, 14, 15, 16, 17, 18, 19, 20, 21, or 22 amino acid truncation. In some embodiments, the Aβ peptide variant having at least 85, 90, 91, 92, 93, 94, 95, 96, 97, 98, or 99% identity with or comprising SEQ ID NO:4 comprises a C-terminal truncation. In some embodiments, the C-terminal truncation comprises a 1, 2, 3, 4, 5, 6, 7, 8, 9, 10, 11, 12, 13, 14, 15, 16, 17, 18, 19, 20, 21, 22, 23, 24, 25, 26, 27, or 28 amino acid truncation. In some embodiments, the fragment or functional derivative of SEQ ID NO:4 comprises an amino acid sequence having at least 85, 86, 87, 88, 89, 90, 91, 92, 93, 94, 95, 96, 97, 98, or 99 sequence identity with KLVFPAE (SEQ ID NO:11).

In some embodiments, the Aβ peptide variant comprises an amino acid sequence having at least 85, 90, 91, 92, 93, 94, 95, 96, 97, 98, or 99% identity with SEQ ID NO:5, or a fragment or functional derivative thereof. In some embodiments, the Aβ peptide variant comprises SEQ ID NO:5, or a fragment or functional derivative thereof. In some embodiments, the Aβ peptide variant having at least 85, 90, 91, 92, 93, 94, 95, 96, 97, 98, or 99% identity with or comprising SEQ ID NO:5 comprises a N-terminal truncation. In some embodiments, the N-terminal truncation comprises a 1, 2, 3, 4, 5, 6, 7, 8, 9, 10, 11, 12, 13, 14, 15, 16, 17, 18, 19, 20, 21, or 22 amino acid truncation. In some embodiments, the Aβ peptide variant having at least 85, 90, 91, 92, 93, 94, 95, 96, 97, 98, or 99% identity with or comprising SEQ ID NO:5 comprises a C-terminal truncation. In some embodiments, the C-terminal truncation comprises a 1, 2, 3, 4, 5, 6, 7, 8, 9, 10, 11, 12, 13, 14, 15, 16, 17, 18, 19, 20, 21, 22, 23, 24, 25, 26, 27, or 28 amino acid truncation. In some embodiments, the fragment or functional derivative of SEQ ID NO:5 comprises an amino acid sequence having at least 85, 86, 87, 88, 89, 90, 91, 92, 93, 94, 95, 96, 97, 98, or 99 sequence identity with KLVDFAE (SEQ ID NO:10).

In some embodiments, the Aβ peptide variant comprises an amino acid sequence having at least 85, 90, 91, 92, 93, 94, 95, 96, 97, 98, or 99% identity with SEQ ID NO:6, or a fragment or functional derivative thereof. In some embodiments, the Aβ peptide variant comprises SEQ ID NO:6, or a fragment or functional derivative thereof. In some embodiments, the Aβ peptide variant having at least 85, 90, 91, 92, 93, 94, 95, 96, 97, 98, or 99% identity with or comprising SEQ ID NO:6 comprises a N-terminal truncation. In some embodiments, the N-terminal truncation comprises a 1, 2, 3, 4, 5, 6, 7, 8, 9, 10, 11, 12, 13, 14, 15, 16, 17, 18, 19, 20, 21, or 22 amino acid truncation. In some embodiments, the Aβ peptide variant having at least 85, 90, 91, 92, 93, 94, 95, 96, 97, 98, or 99% identity with or comprising SEQ ID NO:6 comprises a C-terminal truncation. In some embodiments, the C-terminal truncation comprises a 1, 2, 3, 4, 5, 6, 7, 8, 9, 10, 11, 12, 13, 14, 15, 16, 17, 18, 19, 20, 21, 22, 23, 24, 25, 26, 27, or 28 amino acid truncation. In some embodiments, the fragment or functional derivative of SEQ ID NO:6 comprises an amino acid sequence having at least 85, 86, 87, 88, 89, 90, 91, 92, 93, 94, 95, 96, 97, 98, or 99 sequence identity with KLVPFAE (SEQ ID NO:12).

In some embodiments of the methods and compositions disclosed herein, the Aβ peptide variant is a polypeptide or a polynucleotide that encodes an Aβ peptide variant. In some embodiments, the vector encodes a polynucleotide that encodes an Aβ peptide variant.

In some embodiments, the vector or polynucleotide encoded by the vector encodes the Aβ peptide variant or a fragment or functional derivative thereof. In some embodiments, the vector or polynucleotide encoded by the vector encodes a minigene that encodes the Aβ peptide variant or a fragment or functional derivative thereof. In some embodiments, the minigene that encodes the Aβ peptide variant encodes a nucleotide sequence corresponding to an amino acid sequence comprising a truncated beta-carboxyl-terminal fragment (β-CTF) of amyloid precursor protein. In some embodiments, the truncated β-CTF is fused to a signal peptide sequence. In some embodiments, the signal peptide sequence comprises a nucleotide sequence corresponding to an amino acid sequence comprising a Gaussia luciferase signal peptide or a nucleotide sequence corresponding to an amino acid sequence comprising a mouse immunoglobulin heavy chain signal peptide. In some embodiments, the truncated β-CTF comprises the Aβ peptide variant sequence, a transmembrane domain sequence, and a cytosolic sequence. In some embodiments, the transmembrane domain sequence comprises a nucleotide sequence corresponding to an amino acid sequence comprising SEQ ID NO:9, SEQ ID NO:18, SEQ ID NO:86, or SEQ ID NO:87. In some embodiments, the cytosolic sequence comprises a nucleotide sequence corresponding to an amino acid sequence for membrane anchoring, promotion of gamma-secretase cleavage, and/or extracellular release of Aβ peptide variants. In some embodiments, the cytosolic sequence corresponds to an amino acid sequence comprising two lysine residues. In some embodiments, the cytosolic sequence corresponds to an amino acid sequence comprising three lysine residues. In some embodiments, the cytosolic sequence corresponds to an amino acid sequence comprising in the 5′ to 3′ direction an arginine residue followed by two lysine residues.

In some embodiments, the minigene comprises an amino acid sequence having at least 85, 90, 91, 92, 93, 94, 95, 96, 97, 98, or 99% identity with SEQ ID NO:8, SEQ ID NO:35, SEQ ID NO:36, SEQ ID NO:37, or SEQ ID NO:38. In some embodiments, the minigene comprises SEQ ID NO:8, SEQ ID NO:35, SEQ ID NO:36, SEQ ID NO:37, or SEQ ID NO:38. In some embodiments, the minigene comprises an amino acid sequence having at least 85, 90, 91, 92, 93, 94, 95, 96, 97, 98, or 99% identity with SEQ ID NO:66, SEQ ID NO:67, SEQ ID NO:68, SEQ ID NO:69, or SEQ ID NO:70. In some embodiments, the minigene comprises SEQ ID NO:66, SEQ ID NO:67, SEQ ID NO:68, SEQ ID NO:69, or SEQ ID NO:70. In some embodiments, the minigene comprises an amino acid sequence having at least 85, 90, 91, 92, 93, 94, 95, 96, 97, 98, or 99% identity with SEQ ID NO:71, SEQ ID NO:72, SEQ ID NO:73, SEQ ID NO:74, or SEQ ID NO:75. In some embodiments, the minigene comprises SEQ ID NO:71, SEQ ID NO:72, SEQ ID NO:73, SEQ ID NO:74, or SEQ ID NO:75. In some embodiments, the minigene comprises an amino acid sequence having at least 85, 90, 91, 92, 93, 94, 95, 96, 97, 98, or 99% identity with SEQ ID NO:76, SEQ ID NO:77, SEQ ID NO:78, SEQ ID NO:79, or SEQ ID NO:80. In some embodiments, the minigene comprises SEQ ID NO:76, SEQ ID NO:77, SEQ ID NO:78, SEQ ID NO:79, or SEQ ID NO:80.

In some embodiments, expression of the minigene is regulated by a constitutive promoter. In some embodiments, expression of the minigene is regulated by a tissue-specific or cell-specific promoter. In some embodiments, the cell-specific promoter is a neuron-specific promoter. In some embodiments, the neuron-specific promoter is a synapsin promoter. In some embodiments, the tissue-specific promoter is a choroid plexus-specific promoter. In some embodiments, the tissue-specific promoter is Prlr, Spint2, or F5.

In some embodiments, the polynucleotide that encodes the Aβ peptide or a fragment or functional derivative thereof is comprised in a vector. In some embodiments, the vector is a viral vector or a non-viral vector. In some embodiments, the vector is an adenoviral, lentiviral, retroviral, or adeno-associated viral vector. In some embodiments, the vector is an AAV vector. In some embodiments, the vector is AAV1, AAV2, AAV3, AAV4, AAV5, AAV6, AAV7, AAV8, AAV9, AAV10, AAV11, AAV12, AAV2.5, AAV-DJ, AAVrhlO.XX, AAVrh.8, AAVrh.10, AAVrh.43, AAVpi.2, AAVhu.11, AAVhu.32, AAVhu.37, or PHP.eB AAV. In some embodiments, the vector is AAV9, PHP.eB, AAVrh.8, AAVrh.10, or AAVrh.43.

In some embodiments, a dose of between 1×10⁸ to 1×10¹⁸ vector genomes/kg body weight of the subject is administered to the subject. In some embodiments, a dose of about 1×10¹¹ to about 1×10¹⁴ vector genomes/kg body weight of the subject is administered to the subject. In some embodiments, a dose of about 1×10¹² to about 1×10¹⁵ vector genomes/kg body weight of the subject is administered to the subject. In some embodiments, the vector transduces cells of the subject, and the cells of the subject express the minigene.

In some embodiments of the methods and compositions disclosed herein, the composition further comprises a pharmaceutically acceptable carrier. In some embodiments, the pharmaceutically acceptable carrier comprises liposomes, polymeric micelles, microspheres, or nanoparticles.

In some embodiments, the composition is delivered systemically or locally. In some embodiments, the composition is delivered to the central nervous system systemically via peripheral injection. In some embodiments, the peripheral injection is intravenous injection. In some embodiments, the composition is delivered to cerebrospinal fluid (CSF). In some embodiments, the composition is delivered to the CSF by nonsurgical injection. In some embodiments, nonsurgical injection into the CSF comprises nonsurgical intrathecal injection. In some embodiments, the composition is delivered to the CSF by neurosurgical injection. In some embodiments, neurosurgical injection into the CSF comprises neurosurgical injection into the cisterna magna. In some embodiments, the composition is delivered to the ventricular system. In some embodiments, the composition is delivered to the ventricular system by neurosurgical injection. In some embodiments, the composition crosses the blood-brain barrier.

In some embodiments, the composition is delivered to the subject a single time. In some embodiments, the composition is delivered before onset of Aβ peptide oligomer, protofibril, or fibril formation. In some embodiments, the composition is delivered after onset of Aβ peptide oligomer, protofibril, or fibril formation. In some embodiments, the composition is delivered before onset of amyloid plaque formation. In some embodiments, the composition is delivered after onset of amyloid plaque onset.

In some embodiments, the subject is provided an effective amount of one or more additional therapies for the neurodegenerative disease, disorder, or condition. In some embodiments, the one or more additional therapies comprise Alzheimer's disease medications. In some embodiments, the Alzheimer's disease medications comprise aducanumab, donepezil, rivastigmine, galantamine, memantine, or tacrine.

“Individual, “subject,” and “patient” are used interchangeably and can refer to a human or non-human.

Throughout this application, the term “about” is used to indicate that a value includes the inherent variation of error for the measurement or quantitation method.

The use of the word “a” or “an” when used in conjunction with the term “comprising” may mean “one,” but it is also consistent with the meaning of “one or more,” “at least one,” and “one or more than one.”

The phrase “and/or” means “and” or “or”. To illustrate, A, B, and/or C includes: A alone, B alone, C alone, a combination of A and B, a combination of A and C, a combination of B and C, or a combination of A, B, and C. In other words, “and/or” operates as an inclusive or.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. Compositions and methods “consisting essentially of” any of the ingredients or steps disclosed limits the scope of the claim to the specified materials or steps which do not materially affect the basic and novel characteristic of the claimed invention.

Any method in the context of a therapeutic, diagnostic, or physiologic purpose or effect may also be described in “use” claim language such as “Use of” any compound, composition, or agent discussed herein for achieving or implementing a described therapeutic, diagnostic, or physiologic purpose or effect.

It is contemplated that any embodiment discussed in this specification can be implemented with respect to any method or composition of the invention, and vice versa. Furthermore, compositions of the invention can be used to achieve methods of the invention.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1E show that variant Aβ peptides diminish fibrillization and cytotoxicity of wild-type Aβ. FIG. 1A shows thioflavine-T (ThT) assays for self-aggregation of variant peptides compared to wild-type (WT) Aβ42. None of the 5 variants displayed self-aggregation during the reaction; for comparison, WT Aβ42 (green) reached plateau ThT binding within 1 hr. FIG. 1B shows competition assays testing inhibition of WT Aβ42 aggregation. Each variant was mixed 1:1 with WT Aβ42 and incubated with ThT. V18P exacerbated aggregation of WT peptide; the other four variants abated fibrillization. F20P (red) and F19D/L34P (blue) prevented aggregation and were selected for further study. FIG. 1C shows fibril disassembly assays for F19D/L34P. WT Aβ42 fibrils were exposed to varying concentrations of monomeric F19D/L34P peptide. Compared with fibrils alone, incubation with F19D/L34P produced a concentration-dependent reduction of ThT fluorescence, consistent with disassembly of Aβ fibrils. FIG. 1D shows fibril disassembly for F20P, as described for F19D/L34P. F20P also produced a concentration-dependent reduction of ThT fluorescence, but was considerably less effective than F19D/L34P. FIG. 1E shows MTS assays, which show that unlike wild-type Aβ42 oligomers (green), neither F20P (red) or F19D/L34P (blue) caused toxicity of N2a cells on their own; both were similar to untreated controls (black). In contrast, equimolar co-incubation of WT Aβ with either variant during oligomeric Aβ formation diminished subsequent N2a cytotoxicity. X-axis values represent the volume (μl) of the 100 μl oligomeric Aβ reaction that was added to N2a media for testing. This equates to 0.1 or 10 μM of monomeric WT Aβ42 starting material, plus an equivalent amount of variant peptide where indicated. ANOVA, *p<0.05, ** p<0.01 Bonferroni post-test for comparison with WT Aβ42 alone. All data are presented as mean±SEM.

FIGS. 2A-2D show an expression construct optimized for efficient secretion of variant Aβ. FIG. 2A shows a sequential deletion strategy used to identify the shortest CTF fragment sufficient for γ-secretase cleavage. Constructs were transfected into N2a cells and Aβ was harvested from the media to measure secretion. The optimal construct contained the full transmembrane domain plus two intracellular lysines (KK). This minigene achieved Aβ secretion equivalent to full-length CTF (left blot, arrows indicate secreted Aβ vs. uncleaved Aβ+residual transmembrane (TM) domain for each construct indicated by its ending residues). Secretion of wild-type Aβ42 using the ‘KK’ minimal construct was blocked in a dose-dependent manner by β-secretase inhibitor LY411575 (right blot, arrows again indicate secreted Aβ vs. uncleaved Aβ+TM domain, GSI concentrations in nM). FIG. 2B shows design of the AAV vector for delivery of variant Aβ peptides in vivo. The expression cassette contains the Gaussia luciferase signal peptide at the N-terminus of Aβ, followed by the Aβ42 variant, and the minimal APP C-terminal transmembrane sequence, all controlled by the CAG promoter. FIG. 2C shows extracellular release of full-length variant Aβ using mass spectrometry analysis of immunoprecipitated Aβ. N2a cells expressing Aβ F20P were used to isolate secreted peptide from the media by 6E10 immunoprecipitation. MS1 spectra of the eluted peptides display the expected mass for Aβ40 F20P and Aβ42 F20P (data not shown). The peak for intact Aβ40 F20P at m/z=856.4441 with a +5 charge state indicates a monoisotopic mass of 4,277.1841 Da, whereas the most abundant isotopomer indicates a mass of 4,279.1851 Da and is within 10 ppm error of the expected mass. FIG. 2D shows MS2 fragmentation of the peptide matching the mass of Aβ40 F20P to confirm its sequence identity, with fragment ion tolerance less than 20 ppm error.

FIGS. 3A-3G demonstrate that neonatal AAV injection produces neuronal expression of variant Aβ. AAV encoding either F19D/L34P or F20P Aβ was injected into the lateral ventricles of wild-type neonatal mice. Three weeks or 7 months later, mice were harvested for immunostaining and/or ELISA analysis. FIG. 3A shows that anti-human Aβ immunostaining (6E10, green) demonstrates widespread viral expression in the cortex of this sagittal section harvested 3 wks after P0 injection of AAV-F20P. FIG. 3B shows that co-staining for virally-delivered variant Aβ ((F20P; 6E10, green) and endogenous mouse APP (Y188, red) demonstrate good concordance, suggesting membrane delivery of variant peptide in cortical neurons. FIG. 3C shows mass spectrometry of immunoprecipitated Aβ to confirm the production of full-length variant Aβ in vivo. Mice expressing Aβ F20P were used to isolate peptide from brain homogenate by 6E10 immunoprecipitation. MS1 spectra of the eluted peptides display the expected mass for Aβ42 F20P (shown here) and Aβ40 F20P. The peak for intact Aβ42 F20P at m/z=893.2675 with a +5 charge state (d=0.2) indicates a monoisotopic mass of 4,461.3011 Da, whereas the most abundant isotope configuration was 4,463.3136 Da and is within 15 ppm error of the expected mass. FIG. 3D shows MS2 fragmentation of the peptide to confirm the sequence identity of Aβ42 F20P, with a fragment ion tolerance of 20 ppm error. FIG. 3E shows human Aβ detected by ELISA in the soluble fraction of frontal cortex homogenates from wild-type mice euthanized at 3 weeks of age. Both variants produced human Aβ42, however Aβ40 was only detected in mice transduced with F20P. F20P produced high levels of Aβ40 relative to non-injected mice, while F19D/L34P production of Aβ40 appeared negligible, likely due to poor detection of the variant peptide (abbreviated F19D in graph). Aβ42 production was lower than Aβ40, as expected for constructs encoding a wild-type γ-secretase site. FIG. 3F. Expression of variant human Aβ could still be detected by ELISA 7.5 months after P0 viral injection. Values are for soluble fraction of frontal cortex homogenates from non-transgenic animals. Note that the absolute values of panels C and D cannot be directly compared as the assays were performed at different times using kits from different manufacturing lots. FIG. 3G. The ratio of Aβ40:42 produced by each variant remained constant between 3 weeks (upper panel) and 7.5 months (lower panel). ANOVA, * p<0.05, *** p<0.001, **** p<0.0001. Data are presented as mean±SEM. n=2-10/treatment.

FIGS. 4A-4C demonstrate lifelong expression of variant Aβ reduces plaque load and Aβ accumulation in APP/PS1 mice. APP/PS1 mice were injected at P0 with AAV encoding Aβ F19D/L34P or F20P and harvested 7.5 mo later. FIG. 4A shows that Aβ immunostain reveals decreased plaque accumulation in mice treated with variant Aβ peptide. FIG. 4B shows that cortical plaque load measured as % Aβ area confirms that F20P mice harbored less amyloid than untreated mice. n=5 uninjected, n=4 F19D/L34P, n=8 F20P. FIG. 4C shows that MSD ELISA for human Aβ peptide in guanidine extracts of cortical tissue echoes the plaque histology. Aβ40 levels were reduced by both variants, while F20P also reduced Aβ42 levels. Reduction in Aβ reached significance for F20P and showed a trend for F19D/L34P. n=8 uninjected, n=5 F19D/L34P, n=12 F20P. ANOVA, * p<0.05, ** p<0.01, *** p<0.001, **** p<0.0001. Data are presented as mean±SEM.

FIGS. 5A-5B demonstrate use of a viral serotype to control the spatial distribution and timing of transgene expression. FIG. 5A shows PHP.eB virus encoding red fluorescent protein used to demonstrate CNS expression following peripheral injection. 1.6×10¹¹ particles of PHP.eB encoding CAG-tdTomato was injected into the retro-orbital sinus at 3 mo of age; mice were harvested 2 weeks later. FIG. 5B shows that unlike AAV8 used in FIG. 3, low titer AAV1 preferentially transduces ependymal cells when injected into the lateral ventricle of neonatal mice. Note viral spread into the fourth ventricle. Inset shows lateral ventricle (boxed) at higher magnification. Alternative serotypes such as AAV4 or AAV5 could also be used to provide specificity for ependymal cells when injected into the lateral ventricle.

FIGS. 6A-6D show that variant Aβ peptide diminishes reactive gliosis commensurate with plaque load. Both APP/PS1 and wild-type (non-transgenic, NTG) mice were harvested 7.5 mo after icv P0 viral injection to deliver F20P variant Aβ. Uninjected siblings were used for comparison. FIGS. 6A, 6B show GFAP immunostaining used to detect astrocytes; Iba1 to detect microglia. Fluorescent immunostaining shown in the bottom row was counterstained with thioflavin-S to detect amyloid plaques. Both the size of glial foci and the number of surrounding cells were decreased by F20P treatment in APP/PS1 mice compared with uninjected animals (upper and lower rows). In contrast, viral injection had no impact on microglial morphology or density in NTG mice, but increased the number of GFAP+ cells along the corpus callosum (middle row). FIG. 6C shows quantification of the colorimetric immunostains for GFAP and Iba1 and confirms the qualitative findings that F20P treatment diminished the area of glial staining commensurate in APP/PS1 mice, but elevated GFAP levels in NTG mice compared with uninjected controls. FIG. 6D. Viral expression of human Aβ was detected in a subset of forebrain astrocytes. Co-immunofluorescence for human Aβ (6E10, green) and GFAP (red) detected sparse co-labeled cells (arrows) in NTG mice injected with F20P. APP/PS1 n=5 uninjected, n=7-8 F20P; NTG n=5-6 uninjected, n=5 F20P. A small portion of GFAP+ astrocytes (red) co-labeled with anti-human Aβ antibody 6E10 (green), suggesting they had been transduced at P0 by AAV injection. 2-way ANOVA, * p<0.05, ** p<0.01, **** p<0.0001. Data are presented as mean±SEM.

### DETAILED DESCRIPTION

The present disclosure is based, at least in part, on the surprising discovery that some amyloid beta (Aβ) peptide variants, vectors encoding the variants, or vectors encoding minigenes that enable protein expression of variant Aβ peptides can inhibit aggregation of endogenous Aβ peptide. Further, administering vectors encoding the variants or vectors encoding minigenes that enable protein expression of variant Aβ peptides was surprisingly found to prevent or decrease formation of endogenous Aβ peptide oligomers, protofibrils, fibrils, or plaques and the cytotoxicity of endogenous formation of endogenous Aβ peptide aggregate. As disclosed herein, administration of a therapeutically effective amount of a composition comprising a vector encoding an Aβ peptide variant or a vector encoding a minigene that enables protein expression of variant Aβ peptides can prevent or decrease protein misfolding, endogenous Aβ peptide aggregation, amyloid plaque formation, neuroinflammation, neurodegeneration, neuronal loss, or synaptic loss; decrease tau levels, phosphorylation of tau, or phosphorylated tau levels; slow seeding of tau or seeding of endogenous Aβ peptide; and/or promote cognitive improvement.

Accordingly, in some embodiments, disclosed are methods and compositions for treating or preventing a neurodegenerative disease, disorder, or condition in a subject or inhibiting aggregation of endogenous Aβ peptide in vivo comprising administering a therapeutically effective amount of a composition comprising a vector encoding an Aβ peptide variant or a vector encoding a minigene that enables protein expression of variant Aβ peptides. In some embodiments, the neurodegenerative disease, disorder, or condition is Alzheimer's disease, Parkinson's disease, Parkinson's disease dementia, vascular dementia, cerebral amyloid angiopathy, dementia with Lewy bodies, chronic traumatic encephalopathy, Down syndrome, and/or pathological aging. In some embodiments, the neurodegenerative disease, disorder, or condition is Alzheimer's disease. In some embodiments, the subject is or was previously diagnosed with the neurodegenerative disease, disorder, or condition, symptoms of the neurodegenerative disease, disorder, or condition, or risk of having the neurodegenerative disease, disorder, or condition.

### I. Neurodegenerative Diseases, Disorders, and Conditions

In some embodiments, disclosed are methods for treating neurodegenerative diseases. As used herein, the terms “neurodegenerative disease, disorder, or condition” or “neurodegenerative disease” refer to conditions which primarily affect the neurons in the human brain, resulting in the progressive loss of structure or function of neurons, including death of neurons. The neurodegenerative disease may specifically be of the following types, though it is not limited to these: Alzheimer's disease, Parkinson's disease, Parkinson's disease dementia, vascular dementia, cerebral amyloid angiopathy, dementia with Lewy bodies, chronic traumatic encephalopathy, Down syndrome, and/or pathological aging. In some embodiments, the neurodegenerative disease is Alzheimer's disease.

Neurodegenerative diseases are incurable and debilitating conditions that result in progressive degeneration and/or death of nerve cells in the brain or peripheral nervous system. This causes problems with movement, or mental functioning (called dementias). Dementias are the loss of cognitive functioning, for example, thinking, remembering, and reasoning, and behavioral abilities to such an extent that it interferes with a person's daily life and activities. Dementia ranges in severity from the mildest stage, when it is just beginning to affect a person's functioning, to the most severe stage, when the person must depend completely on others for basic activities of daily living.

The causes of dementia can vary depending on the types of brain changes that may be taking place. Dementias are responsible for the greatest burden of neurodegenerative diseases, with Alzheimer's disease representing approximately 60-70% of dementia cases. In 2016, an estimated 5.4 million Americans were living with Alzheimer's disease. Other dementias include Lewy body dementia, frontotemporal disorders, and vascular dementia. In some cases, patients may have mixed dementia comprising a combination of two or more types of dementia. For example, some patients have both Alzheimer's disease and vascular dementia.

Alzheimer's disease (AD) is a chronic neurodegenerative disease that results in loss of neurons and synapses in the cerebral cortex and certain subcortical structures, resulting in gross atrophy and degeneration of the temporal lobe, parietal lobe, and parts of the frontal cortex and cingulate gyrus. Degeneration may also be present in brainstem nuclei like the locus coeruleus. Studies using MRI and PET have documented reductions in the size of specific brain regions in patients with AD as they progressed from mild cognitive impairment to Alzheimer's disease, and in comparison with similar images from healthy older adults. AD pathology is primarily characterized by the presence of senile plaques and neurofibrillary tangles, which disrupt normal brain function and chemistry and lead to a significant deficit of neurotransmitters, resulting in a progressive loss of brain function. Abnormal amounts of beta amyloids and tau proteins form in the brain and begin to encroach on brain cells, forming plaques and neurofibrillary tangles. Both amyloid plaques and neurofibrillary tangles are clearly visible by microscopy in brains of those afflicted by AD, especially in the hippocampus. Although many older individuals develop some plaques and tangles as a consequence of aging, the brains of patients with AD have a greater number of them in specific brain regions such as the temporal lobe.

The most common early symptom is difficulty remembering recent events. As AD advances, symptoms can include problems with language, disorientation, mood swings, loss of motivation, mismanagement of self-care, and behavioral issues. Patients with more severe AD often withdraw from family and society. Gradually, bodily functions are lost, which ultimately leads to death. Although the speed of progression can vary, the typical life expectancy following diagnosis of AD is three to nine years.

A. Alzheimer's Disease Course

The disease course is divided into distinct stages, with a progressive pattern of cognitive and functional impairment. Preclinical AD is characterized by the presence of biomarkers for AD, such as amyloids detected by PET or CSF testing. Mild cognitive impairment is characterized by the onset of AD symptoms. This is often found to be a transitional stage between normal aging and dementia. MCI can present with a variety of symptoms, and when memory loss is the predominant symptom, it is termed “amnestic MCI” and is frequently seen as a prodromal stage of Alzheimer's disease. The first symptoms are often mistakenly attributed to aging or stress, but detailed neuropsychological testing can reveal mild cognitive difficulties up to eight years before clinical criteria for diagnosis of AD are fulfilled. The most noticeable deficit is short term memory loss, which shows up as difficulty in remembering recently learned facts and an inability to acquire new information. Subtle problems with executive functions of attentiveness, planning, flexibility, and abstract thinking, or impairments in semantic memory, the memory of meanings and concept relationships, can also be symptomatic of early stage AD. Apathy can also be observed and remains the most persistent neuropsychiatric symptom throughout the course of the disease. Depressive symptoms, irritability, and reduced awareness of subtle memory difficulties are also common.

In patients with AD, the increasing impairment of learning and memory eventually leads to a definitive diagnosis. In a small percentage of patients, difficulties with language, executive functions, perception (agnosia), or execution of movements (apraxia) are more prominent than memory problems. AD does not affect all memory capacities equally. Older memories of the person's life (episodic memories), facts learned (semantic memory), and implicit memory (the memory of the body on how to do things) are affected to a lesser degree than new facts or memories. Language problems are mainly characterized by a shrinking vocabulary and decreased word fluency, leading to a general deficits in oral and written language. In this stage, basic ideas can usually be adequately communicated. Certain movement coordination and planning difficulties, or apraxia, may be present while performing fine motor tasks, but they can be unnoticed. As AD progresses, patients may continue to perform many tasks independently, but may need assistance or supervision with the most cognitively demanding activities.

Progressive deterioration eventually hinders independence, with patients being unable to perform most common activities of daily living. Speech difficulties may become evident due to an inability to recall vocabulary, which leads to frequent incorrect word substitutions, or paraphasias. Reading and writing skills may be progressively lost. Complex motor sequences may become less coordinated as AD progresses, so falling risk increases. Memory problems worsen, and long-term memory may become impaired. Behavioral and neuropsychiatric changes may become more prevalent and can include wandering, irritability, and labile affect leading to crying, outbursts of unpremeditated aggression, or resistance to caregiving. Sundowning can also appear. Approximately 30% of patients with AD develop illusionary misidentifications and other delusional symptoms. Patients can also lose insight of their disease process and limitations, known as anosognosia.

During the final stages of AD, the patient may be completely dependent upon caregivers. Language may be reduced to simple phrases or even single words, eventually leading to complete loss of speech. Aggressiveness can still be present, as can extreme apathy and exhaustion. Muscle mass and mobility may deteriorate to the point where patients are bedridden and unable to feed themselves. The cause of death is usually an external factor, such as infection or pneumonia, rather than the disease itself.

B. Causes of Alzheimer's Disease

The genetic heritability of Alzheimer's disease, based on reviews of twin and family studies, ranges from 49% to 79%. Around 0.1% of the cases are familial forms of autosomal dominant inheritance, which have an onset before age 65, known as early onset familial Alzheimer's disease. Most autosomal dominant familial AD cases can be attributed to mutations in one of three genes: those encoding amyloid precursor protein (APP) and presenilins 1 and 2. Most mutations in the APP and presenilin genes increase the production of beta-amyloid protein (A-beta protein or Aβ protein), which is the main component of senile plaques, or increase the ratio of Aβ42 protein (consisting of 42 amino acids) to Aβ40 protein (consisting of 40 amino acids). Some of mutations alter the ratio between Aβ42 and the other major forms of amyloid beta protein, such as Aβ40, without increasing total Aβ levels.

Most cases of Alzheimer's disease do not exhibit autosomal-dominant inheritance and are termed sporadic AD, in which environmental and genetic differences may act as risk factors. The best known genetic risk factor is the inheritance of the F4 allele of apolipoprotein E (APOE). Between 40 and 80% of patients with AD possess at least one APOEε4 allele. The APOEε4 allele increases the risk of the disease by three times in heterozygotes and by 15 times in homozygotes. Genome-wide association studies (GWAS) have found more than 30 areas in additional genes that appear to affect AD risk. These genes include but are not limited to: ABCA7, SORL1, CASS4, CELF1, FERMT2, HLA-DRB5, INPP5D, MEF2C, NME8, PTK2B, SORL1, ZCWPW1, SLC24A4, CLU, PICALM, CR1, BIN1, MS4A, ABCA7, EPHA1, and CD2Aβ. Additional genes associated with AD risk can be found in B. W. Kunkle et al., Nature Genetics 51, 414-430 (2019), specifically incorporated by reference herein in its entirety. Alleles in the TREM2 gene have also been associated with a 3 to 5 fold higher risk of developing AD. In some TREM2 variants, microglia in the brain are no longer able to control the amount of beta amyloid present. Many single-nucleotide polymorphisms (SNPs) are associated with Alzheimer's, with a 2018 study by Mukherjee, et al. (Genetic data and cognitively defined late-onset Alzheimer's disease subgroups. Mol Psychiatry (2018)), specifically incorporated by reference herein in its entirety, adding 30 SNPs by differentiating AD into 6 categories, including memory, language, visuospatial, and executive functioning.

A Japanese pedigree of familial Alzheimer's disease was found to be associated with a deletion mutation of codon 693 of APP, known as the Osaka mutation. Homozygotes with this mutation develop Alzheimer's disease. The mutation accelerates Aβ oligomerization but the proteins do not form amyloid fibrils. Mice expressing this mutation have all the usual pathologies of Alzheimer's disease caused by the Osaka mutation.

An alanine to valine substitution mutation at codon 673 in the APP gene increases the risk for Alzheimer disease in homozygote carriers, with the development of amyloid fibrils, possibly by promoting the formation of Aβ, while heterozygote carriers of A673V may be protected against Alzheimer's disease. This substitution is adjacent to the beta secretase cleavage site and can result in an increase in APP cleavage by beta-secretase through the amyloidogenic pathway. Substitution of the same alanine at position 673 to a threonine (A673T), in contrast, can protect against Alzheimer's disease. This substitution is adjacent to the beta secretase cleavage site and can result in a 40% reduction in the formation of amyloid beta in vitro.

It has also been theorized that extracellular Aβ deposits are the fundamental cause of AD. Support for this theory comes from the location of the gene for APP on chromosome 21, together with the fact that many patients with trisomy 21 (Down Syndrome) who have an extra gene copy exhibit at least the earliest symptoms of AD by 40 years of age. Also, APOE4 is a major genetic risk factor for AD. See, e.g., M. Safieh et al., BMC Medicine 17:64 (2019), and C. G. Fernandez et al., Front. Aging Neuroscience 11:14 (2019), specifically incorporated by reference herein in their entirety. Transgenic mice that express a mutant form of the human APP gene develop fibrillar amyloid plaques and Alzheimer's-like brain pathology with spatial learning deficits.

Aβ deposits are formed from Aβ peptides (also known as A-beta peptides or beta-amyloids), which are typically 39-43 amino acids in length. Aβ peptides are fragments from a larger protein called amyloid precursor protein (APP), a transmembrane protein that penetrates through the cell's membrane. APP appears to play roles in normal neuron growth, survival, and post-injury repair. In Alzheimer's disease, gamma secretase and beta secretase act together in a proteolytic process which causes APP to be divided into smaller fragments. These fragments give rise to fibrils of beta-amyloid which can self-assemble into the dense extracellular deposits known as senile plaques or amyloid plaques. Plaques are dense, mostly insoluble deposits of beta-amyloid peptide and cellular material outside and around neurons. An experimental vaccine was found to clear the amyloid plaques in early human trials, but it did not have any significant effect on dementia, which may indicate that non-plaque Aβ oligomers are the primary pathogenic form of Aβ. These toxic oligomers, also referred to as amyloid-derived diffusible ligands, bind to a surface receptor on neurons and change the structure of the synapse, thereby disrupting neuronal communication.

Other studies suggest that tau protein abnormalities initiate the AD cascade. Every neuron has a cytoskeleton partly made up of microtubules. Tau protein stabilizes the microtubules when phosphorylated. In this model of AD, tau undergoes chemical changes, becoming hyperphosphorylated. Hyperphosphorylated tau begins to pair with other threads of tau. Eventually, they form neurofibrillary tangles inside nerve cell bodies. Tangles (neurofibrillary tangles) are aggregates of the microtubule-associated protein tau which has become hyperphosphorylated and accumulates inside cells. When this occurs, the microtubules disintegrate, destroying the structure of the cell's cytoskeleton which collapses the neuron's transport system. This may result first in malfunctions in biochemical communication between neurons and later in the death of the cells. Pathogenic tau may also cause neuronal death through transposable element dysregulation.

Age is a significant risk factor for AD. Age-related changes in the brain may harm neurons and affect other types of brain cells to contribute to Alzheimer's damage. These age-related changes include atrophy (shrinking) of certain parts of the brain, inflammation, vascular damage, production of unstable molecules called free radicals, and mitochondrial dysfunction (a breakdown of energy production within a cell). Thus, advanced (more than 60 years old) age is a risk factor for AD.

There are several additional theories which have been presented to explain the cause of AD. For example, an inflammatory hypothesis is that AD is caused due to a self-perpetuating progressive inflammation in the brain culminating in neurodegeneration. In some models, infectious viral and microbial agents are believed to work in tandem with Aβ to produce a positive feedback mechanism for neuroinflammation. See, e.g., A. L. Komaroff, JAMA 324(3):239-240 (2020). A neurovascular hypothesis has also been proposed which provides that poor functioning of the blood-brain barrier may be involved. The cellular homeostasis of biometals such as ionic copper, iron, and zinc is also disrupted in AD; these ions affect and are affected by tau, APP, and APOE, and their dysregulation may cause oxidative stress that may contribute to the pathology. Another hypothesis posits that dysfunction of oligodendrocytes and their associated myelin during aging contributes to axon damage, which then causes amyloid production and tau hyper-phosphorylation as a side effect.

C. Diagnosis of Alzheimer's Disease

Alzheimer's disease is usually diagnosed based on patients' medical history, history from relatives, and behavioral observations. The presence of characteristic neurological and neuropsychological features and the absence of alternative conditions is supportive. Advanced medical imaging with computed tomography or magnetic resonance imaging and single-photon emission computed tomography or positron emission tomography can be used to help exclude other cerebral pathology or subtypes of dementia. Moreover, it may predict conversion from prodromal stages typified by mild cognitive impairment to Alzheimer's disease.

To diagnose AD, the National Institute on Aging and the Alzheimer's Association Workgroup's Research Framework uses a biomarker classification scheme that divides the current major AD biomarkers into three categories, based on the type of pathologic change each measures: β-amyloid (A), pathological tau (T), and neurodegeneration (N). The ATN nomenclature represents a conceptual framework that is based on the past decade's empiric observations of relationships between markers of amyloid, tau, and neurodegeneration. “A” refers to amyloid R (Aβ) as measured either by amyloid positron emission tomography (PET) imaging of amyloid plaques or in the cerebrospinal fluid (CSF) as Aβ42 or the Aβ42 to Aβ40 ratio. “T” refers to tau pathology as measured by CSF phosphorylated tau or tau PET imaging of parenchymal neurofibrillary tangles. “N” refers to neurodegeneration or neuronal injury and dysfunction, as measured for example by hippocampal volume or cortical volume or thickness. While “A” plus “T” is considered to have diagnostic specificity for AD, “N” is not specific for AD diagnoses because it can reflect any number of etiologies in addition to AD. The ATN system is described in detail in D. S. Knopman et al., Alzheimer's & Dement. 14(4):563-575 (2018), specifically incorporated by reference herein in its entirety.

Assessment of intellectual functioning including memory testing can further characterize the state of the disease. Medical organizations have created diagnostic criteria to standardize the diagnostic process. In 1984, the National Institute of Neurological and Communicative Disorders and Stroke (NINCDS) and the Alzheimer's Disease and Related Disorders Association (ADRDA, now known as the Alzheimer's Association) established the most commonly used NINCDS-ADRDA Alzheimer's Criteria, which require that the presence of cognitive impairment, and a suspected dementia syndrome, be confirmed by neuropsychological testing for a clinical diagnosis of possible or probable AD. Eight intellectual domains are most commonly impaired in AD—memory, language, perceptual skills, attention, motor skills, orientation, problem solving and executive functional abilities. These domains are equivalent to the NINCDS-ADRDA Alzheimer's Criteria as listed in the Diagnostic and Statistical Manual of Mental Disorders (DSM-IV-TR) published by the American Psychiatric Association. Good statistical reliability and validity have been shown between the diagnostic criteria and definitive histopathological confirmation.

In 2011, the National Institute on Aging/Alzheimer's Association released updated approaches for clinicians and scientists to provide more advanced guidelines for moving forward with research on diagnosis and treatments. The 2011 guidelines differ from the 1984 diagnostic criteria in a few key ways. They recognize that Alzheimer's disease progresses on a spectrum with three stages—an early, preclinical stage with no symptoms; a middle stage of mild cognitive impairment; and a final stage marked by symptoms of dementia. The 1984 criteria addressed only one stage of disease—the final stage of dementia; expand the criteria for Alzheimer's dementia beyond memory loss as the first or only major symptom. They recognize that other aspects of cognition, such as word-finding ability or judgment, may become impaired first. The 1984 criteria focused on memory loss as the central emerging characteristic of Alzheimer's dementia; reflect a better understanding of the distinctions and associations between Alzheimer's and non-Alzheimer's dementias, as well as between Alzheimer's and disorders that may influence its development, such as vascular disease. In 1984, these relationships were not well recognized or understood; and recognize the potential use of biomarkers—indicators of underlying brain disease—to diagnose Alzheimer's disease. However, the guidelines state that biomarkers are almost exclusively to be used in research rather than in a clinical setting. These biomarkers did not exist when the original criteria were developed in 1984, and confirmation of the diagnosis commonly by autopsy after death.

Neuropsychological tests such as the mini-mental state examination (MMSE) are widely used to evaluate the cognitive impairments needed for diagnosis. Neurological examination in early AD will usually provide normal results, except for obvious cognitive impairment, which may not differ from that resulting from other diseases processes, including other causes of dementia. Further neurological examinations are crucial in the differential diagnosis of AD and other diseases. Interviews with family members are also utilized in the assessment of the disease. Caregivers can supply important information on the daily living abilities, as well as on the decrease, over time, of the person's mental function. A caregiver's viewpoint can be particularly important, since a person with AD is commonly unaware of his own deficits.

Supplemental testing can provide extra information on features of the disease or can be used to rule out other diagnoses. Blood tests can identify other causes for dementia than AD. It is also common to perform thyroid function tests, assess B12, rule out syphilis, rule out metabolic problems (including tests for kidney function, electrolyte levels and for diabetes), assess levels of heavy metals (e.g., lead, mercury), and anemia. Psychological tests for depression may also be employed, since depression can either be concurrent with AD, an early sign of cognitive impairment, or even the cause.

### II. Aβ Peptide Variants

In some embodiments, the disclosed compositions comprise at least one proteinaceous molecule. In some embodiments, the proteinaceous molecule comprises an amyloid beta (Aβ) peptide or variant thereof. In some embodiments, the proteinaceous molecule comprising a wild-type Aβ peptide or an Aβ peptide variant or a fragment or functional derivative thereof is encoded by a polynucleotide. In some embodiments, the polynucleotide is encoded by a vector. In some embodiments, the polynucleotide is encoded by a vector encoding a minigene that enables protein expression of Aβ peptides. Thus, in some embodiments, the wild-type or variant Aβ peptides or fragments or functional derivatives thereof are encoded by a vector. In some embodiments, the Aβ peptides or fragments or functional derivatives thereof are encoded by a vector encoding a minigene that enables protein expression of Aβ peptides.

Aβ peptides are fragments from a larger protein called amyloid precursor protein (APP), a transmembrane protein that penetrates through the membrane of neurons. A representative mRNA APP sequence can be found at GenBank® Accession No. NM_000484 and comprises the following amino acid sequence:

(SEQ ID NO: 1)
MLPGLALLLLAAWTARALEVPTDGNAGLLAEPQIAMFCGRLNMHMNVQ
NGKWDSDPSGTKTCIDTKEGILQYCQEVYPELQITNVVEANQPVTIQNW
CKRGRKQCKTHPHFVIPYRCLVGEFVSDALLVPDKCKFLHQERMDVCET
HLHWHTVAKETCSEKSTNLHDYGMLLPCGIDKFRGVEFVCCPLAEESDN
VDSADAEEDDSDVWWGGADTDYADGSEDKVVEVAEEEEVAEVEEEEADD
DEDDEDGDEVEEEAEEPYEEATERTTSIATTTTTTTESVEEVVREVCSE
QAETGPCRAMISRWYFDVTEGKCAPFFYGGCGGNRNNFDTEEYCMAVCG
SAMSQSLLKTTQEPLARDPVKLPTTAASTPDAVDKYLETPGDENEHAHF
QKAKERLEAKHRERMSQVMREWEEAERQAKNLPKADKKAVIQHFQEKVE
SLEQEAANERQQLVETHMARVEAMLNDRRRLALENYITALQAVPPRPRH
VENMLKKYVRAEQKDRQHTLKHFEHVRMVDPKKAAQIRSQVMTHLRVIY
ERMNQSLSLLYNVPAVAEEIQDEVDELLQKEQNYSDDVLANMISEPRIS
YGNDALMPSLTETKTTVELLPVNGEFSLDDLQPWHSFGADSVPANTENE
VEPVDARPAADRGLTTRPGSGLTNIKTEEISEVKMDAEFRHDSGYEVHH
QKLVFFAEDVGSNKGAIIGLMVGGVVIATVIVITLVMLKKKQYTSIHHG
VVEVDAAVTPEERHLSKMQQNGYENPTYKFFEQMQN.

Gamma secretase and beta secretase act together in a proteolytic process which causes APP to be divided into smaller amino acid fragments, known as Aβ (amyloid beta or A-beta) peptides. Gamma secretase, which produces the C-terminal end of the Aβ peptide, cleaves within the transmembrane region of APP and can generate a number of C-terminal fragment isoforms of 30-51 amino acid residues in length. Separate from these C-terminal fragments, 42 amino acid Aβ peptide fragments comprising the sequence DAEFRHDSGYEVHHQKLVFFAEDVGSNKGAIIGLMVGGVVIA (SEQ ID NO:2) are also produced by cleavage of APP. These fragments give rise to fibrils of beta-amyloid which can self-assemble into the dense extracellular deposits known as plaques. Due to its more hydrophobic nature, Aβ42 is the most amyloidogenic form of the peptide. However, the central amino acid sequence KLVFFAE (SEQ ID NO:39) is known to form amyloids on its own and may form the core of amyloid fibrils.

Thus, in some embodiments of the disclosure, the disclosed compositions comprise the wild-type Aβ peptide. In some embodiments of the disclosure, the disclosed compositions comprise variants of the wild-type Aβ peptide. In some embodiments, wild-type Aβ peptide or Aβ peptide variants promote aggregation of endogenous Aβ peptide. In some embodiments, wild-type Aβ peptide or Aβ peptide variants self-assemble into oligomers, protofibrils, fibrils, or plaques. In some embodiments, Aβ peptide variants prevent aggregation of endogenous Aβ peptide. In some embodiments, Aβ peptide variants do not self-assemble into oligomers, protofibrils, fibrils, or plaques. In some embodiments, Aβ peptide variants diminish cytotoxicity of remaining Aβ peptide aggregate. In some embodiments, variants of full-length Aβ disclosed herein allow targeting of multiple aggregation domains simultaneously to increase affinity and specificity for Aβ.

In some embodiments, a wild-type Aβ peptide comprises an amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, or any value derivable therein, with DAEFRHDSGYEVHHQKLVFFAEDVGSNKGAIIGLMVGGVVIA (SEQ ID NO:2), or a fragment or functional derivative thereof. In some embodiments, a wild-type Aβ peptide of the disclosure comprises, consists of, or consists essentially of SEQ ID NO:2, or a fragment or functional derivative thereof. In some embodiments, the fragment or functional derivative of SEQ ID NO:2 comprises an amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, or any value derivable therein, with KLVFFAE (SEQ ID NO:39).

In some embodiments, an Aβ peptide variant comprises an amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, or any value derivable therein, with DAEFRHDSGYEVHHQKLVDFAEDVGSNKGAIIGPMVGGVVIA (SEQ ID NO:3), or a fragment or functional derivative thereof, wherein the bolded residues represent substitutions from the wild-type Aβ peptide. In some embodiments, an Aβ peptide variant of the disclosure comprises, consists of, or consists essentially of SEQ ID NO:3, or a fragment or functional derivative thereof. In some embodiments, the fragment or functional derivative of SEQ ID NO:3 comprises an amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, or any value derivable therein, with KLVDFAE (SEQ ID NO:10).

In some embodiments, an Aβ peptide variant comprises an amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, or any value derivable therein, with DAEFRHDSGYEVHHQKLVFPAEDVGSNKGAIIGLMVGGVVIA (SEQ ID NO:4), or a fragment or functional derivative thereof, wherein the bolded residues represent substitutions from the wild-type Aβ peptide. In some embodiments, an Aβ peptide variant of the disclosure comprises, consists of, or consists essentially of SEQ ID NO:4, or a fragment or functional derivative thereof. In some embodiments, the fragment or functional derivative of SEQ ID NO:4 comprises an amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, or any value derivable therein, with KLVFPAE (SEQ ID NO:11).

In some embodiments, an Aβ peptide variant comprises an amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, or any value derivable therein, with DAEFRHDSGYEVHHQKLVDFAEDVGSNKGAIIGLMVGGVVIA (SEQ ID NO:5), or a fragment or functional derivative thereof, wherein the bolded residues represent substitutions from the wild-type Aβ peptide. In some embodiments, an Aβ peptide variant of the disclosure comprises, consists of, or consists essentially of SEQ ID NO:5, or a fragment or functional derivative thereof. In some embodiments, the fragment or functional derivative of SEQ ID NO:5 comprises an amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, or any value derivable therein, with KLVDFAE (SEQ ID NO:10).

In some embodiments, an Aβ peptide variant comprises an amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, or any value derivable therein, with DAEFRHDSGYEVHHQKLVPFAEDVGSNKGAIIGLMVGGVVIA (SEQ ID NO:6), or a fragment or functional derivative thereof, wherein the bolded residues represent substitutions from the wild-type Aβ peptide. In some embodiments, an Aβ peptide variant of the disclosure comprises, consists of, or consists essentially of SEQ ID NO:6, or a fragment or functional derivative thereof. In some embodiments, the fragment or functional derivative of SEQ ID NO:6 comprises an amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, or any value derivable therein, with KLVPFAE (SEQ ID NO:12).

In some embodiments, an Aβ peptide variant comprises an amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, or any value derivable therein, with DAEFRHDSGYEVHHQKLPFFAEDVGSNKGAIIGLMVGGVVIA (SEQ ID NO:7), or a fragment or functional derivative thereof, wherein the bolded residues represent substitutions from the wild-type Aβ peptide. In some embodiments, an Aβ peptide variant of the disclosure comprises, consists of, or consists essentially of SEQ ID NO:7, or a fragment or functional derivative thereof. In some embodiments, the fragment or functional derivative of SEQ ID NO:7 comprises an amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, or any value derivable therein, with KLPFFAE (SEQ ID NO:65). In some embodiments, the Aβ peptide variant does not comprise, consist of, or consist essentially of SEQ ID NO:7.

In some embodiments, a wild-type Aβ peptide or Aβ peptide variant comprising, consisting of, or consisting essentially of SEQ ID NO:2, SEQ ID NO:3, SEQ ID NO:4, SEQ ID NO:5, SEQ ID NO:6, or SEQ ID NO:7 comprises a N-terminal truncation. In some embodiments, the N-terminal truncation comprises a 1 to 22 amino acid truncation. In some embodiments, the N-terminal truncation comprises a 1, 2, 3, 4, 5, 6, 7, 8, 9, 10, 11, 12, 13, 14, 15, 16, 17, 18, 19, 20, 21, or 22 amino acid truncation.

In some embodiments, a wild-type Aβ peptide or Aβ peptide variant comprising, consisting of, or consisting essentially of SEQ ID NO:2, SEQ ID NO:3, SEQ ID NO:4, SEQ ID NO:5, SEQ ID NO:6, or SEQ ID NO:7 comprises a C-terminal truncation. In some embodiments, the C-terminal truncation comprises a 1 to 27 amino acid truncation. In some embodiments, the C-terminal truncation comprises a 1, 2, 3, 4, 5, 6, 7, 8, 9, 10, 11, 12, 13, 14, 15, 16, 17, 18, 19, 20, 21, 22, 23, 24, 25, 26, 27, or 28 amino acid truncation.

In some embodiments, a wild-type Aβ peptide or Aβ peptide variant comprising, consisting of, or consisting essentially of SEQ ID NO:2, SEQ ID NO:3, SEQ ID NO:4, SEQ ID NO:5, SEQ ID NO:6, or SEQ ID NO:7 comprises a N-terminal truncation and a C-terminal truncation. In some embodiments, the N-terminal truncation comprises a 1 to 22 amino acid truncation, and the C-terminal truncation comprises a 1 to 27 amino acid truncation. In some embodiments, the N-terminal truncation comprises a 1, 2, 3, 4, 5, 6, 7, 8, 9, 10, 11, 12, 13, 14, 15, 16, 17, 18, 19, 20, 21, or 22 amino acid truncation, and the C-terminal truncation comprises a 1, 2, 3, 4, 5, 6, 7, 8, 9, 10, 11, 12, 13, 14, 15, 16, 17, 18, 19, 20, 21, 22, 23, 24, 25, 26, 27, or 28 amino acid truncation.

In some embodiments, a wild-type Aβ peptide or Aβ peptide variant comprises a polypeptide. In some embodiments, a wild-type Aβ peptide or Aβ peptide variant comprises a polynucleotide that encodes the wild-type Aβ peptide or Aβ peptide variant or a fragment or functional derivative thereof. In some embodiments, a vector encodes a polynucleotide that encodes a wild-type Aβ peptide or Aβ peptide variant or a fragment or functional derivative thereof. In some embodiments, the vector or polynucleotide encoded by the vector encodes the wild-type Aβ peptide or Aβ peptide variant or a fragment or functional derivative thereof fused to a signal peptide. In some embodiments, the vector or polynucleotide encoded by the vector encodes the entire APP beta-carboxyl-terminal fragment (β-CTF), comprising the 99 C-terminal amino acids of the amyloid precursor protein which includes a wild-type Aβ peptide or Aβ peptide variant amino acid sequence, fused to a signal peptide at the N-terminus of the R-CTF.

In some embodiments, the vector or polynucleotide encoded by the vector encodes a minigene that encodes a wild-type Aβ peptide or Aβ peptide variant or a fragment or functional derivative thereof. In some embodiments, the minigene encodes a nucleotide sequence corresponding to an amino acid sequence comprising a truncated β-CTF fused to a signal peptide at the N-terminus of the β-CTF. In some embodiments, truncated β-CTF comprises a wild-type Aβ peptide or Aβ peptide variant amino acid sequence (or a fragment or functional derivative thereof) which comprises both extracellular and transmembrane amino acids, a transmembrane domain amino acid sequence, and a cytosolic amino acid sequence.

Thus, in some embodiments, a minigene can encode any signal peptide disclosed herein, a wild-type Aβ peptide or any Aβ peptide variant or a fragment or functional derivative thereof disclosed herein, any transmembrane domain amino acid sequence disclosed herein, and any cytosolic amino acid sequence disclosed herein. In some embodiments, the minigene encodes in the 5′ to 3′ direction a signal peptide disclosed herein, a wild-type Aβ peptide or a Aβ peptide variant or a fragment or functional derivative thereof disclosed herein, a transmembrane domain amino acid sequence disclosed herein, and a cytosolic amino acid sequence disclosed herein.

In some embodiments, the minigene comprises a nucleotide sequence corresponding to an amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, or any value derivable therein, with DAEFRHDSGYEVHHQKLVPFAEDVGSNKGAIIGLMVGGVVIATVIVITLVMLKK (SEQ ID NO:8), DAEFRHDSGYEVHHQKLVPFAEDVGSNKGAIIGLMVGGVVIATVIVITLVMLKKK (SEQ ID NO:35), DAEFRHDSGYEVHHQKLVPFAEDVGSNKGAIIGLMVGGVVIATVIVITLVMAKK K (SEQ ID NO:36), DAEFRHDSGYEVHHQKLVPFAEDVGSNKGAIIGLMVGGVVIATVIVITLVMLRKK (SEQ ID NO:37), DAEFRHDSGYEVHHQKLVPFAEDVGSNKGAIIGLMVGGVVIATVIVITLVMARK K (SEQ ID NO:38), or a fragment or functional derivative thereof, wherein the bolded text corresponds to an Aβ peptide variant amino acid sequence, the underlined text corresponds to a transmembrane domain amino acid sequence, and the unmodified text corresponds to a cytosolic amino acid sequence. In some embodiments, the minigene comprises, consists of, or consists essentially of a nucleotide sequence corresponding to the amino acid sequence of SEQ ID NO:8, SEQ ID NO:35, SEQ ID NO:36, SEQ ID NO:37, or SEQ ID NO:38.

In some embodiments, the minigene comprises a nucleotide sequence corresponding to an amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, or any value derivable therein, with DAEFRHDSGYEVHHQKLVDFAEDVGSNKGAIIGPMVGGVVIATVIVITLVMLKK (SEQ ID NO:66), DAEFRHDSGYEVHHQKLVDFAEDVGSNKGAIIGPMVGGVVIATVIVITLVMLKK K (SEQ ID NO:67), DAEFRHDSGYEVHHQKLVDFAEDVGSNKGAIIGPMVGGVVIATVIVITLVMAKK K (SEQ ID NO:68), DAEFRHDSGYEVHHQKLVDFAEDVGSNKGAIIGPMVGGVVIATVIVITLVMLRKK (SEQ ID NO:69), DAEFRHDSGYEVHHQKLVDFAEDVGSNKGAIIGPMVGGVVIATVIVITLVMARK K (SEQ ID NO:70), or a fragment or functional derivative thereof, wherein the bolded text corresponds to an Aβ peptide variant amino acid sequence, the underlined text corresponds to a transmembrane domain amino acid sequence, and the unmodified text corresponds to a cytosolic amino acid sequence. In some embodiments, the minigene comprises, consists of, or consists essentially of a nucleotide sequence corresponding to the amino acid sequence of SEQ ID NO:66, SEQ ID NO:67, SEQ ID NO:68, SEQ ID NO:68, or SEQ ID NO:70.

In some embodiments, the minigene comprises a nucleotide sequence corresponding to an amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, or any value derivable therein, with DAEFRHDSGYEVHHQKLVFPAEDVGSNKGAIIGLMVGGVVIATVIVITLVMLKK (SEQ ID NO:71), DAEFRHDSGYEVHHQKLVFPAEDVGSNKGAIIGLMVGGVVIATVIVITLVMLKKK (SEQ ID NO:72), DAEFRHDSGYEVHHQKLVFPAEDVGSNKGAIIGLMVGGVVIATVIVITLVMAKK K (SEQ ID NO:73), DAEFRHDSGYEVHHQKLVFPAEDVGSNKGAIIGLMVGGVVIATVIVITLVMLRKK (SEQ ID NO:74), DAEFRHDSGYEVHHQKLVFPAEDVGSNKGAIIGLMVGGVVIATVIVITLVMARK K (SEQ ID NO:75), or a fragment or functional derivative thereof, wherein the bolded text corresponds to an Aβ peptide variant amino acid sequence, the underlined text corresponds to a transmembrane domain amino acid sequence, and the unmodified text corresponds to a cytosolic amino acid sequence. In some embodiments, the minigene comprises, consists of, or consists essentially of a nucleotide sequence corresponding to the amino acid sequence of SEQ ID NO:71, SEQ ID NO:72, SEQ ID NO:73, SEQ ID NO:74, or SEQ ID NO:75.

In some embodiments, the minigene comprises a nucleotide sequence corresponding to an amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, or any value derivable therein, with DAEFRHDSGYEVHHQKLVDFAEDVGSNKGAIIGLMVGGVVIATVIVITLVMLKK (SEQ ID NO:76), DAEFRHDSGYEVHHQKLVDFAEDVGSNKGAIIGLMVGGVVIATVIVITLVMLKK K (SEQ ID NO:77), DAEFRHDSGYEVHHQKLVDFAEDVGSNKGAIIGLMVGGVVIATVIVITLVMAKK K (SEQ ID NO:78), DAEFRHDSGYEVHHQKLVDFAEDVGSNKGAIIGLMVGGVVIATVIVITLVMLRK K (SEQ ID NO:79), DAEFRHDSGYEVHHQKLVDFAEDVGSNKGAIIGLMVGGVVIATVIVITLVMARK K (SEQ ID NO:80), or a fragment or functional derivative thereof, wherein the bolded text corresponds to an Aβ peptide variant amino acid sequence, the underlined text corresponds to a transmembrane domain amino acid sequence, and the unmodified text corresponds to a cytosolic amino acid sequence. In some embodiments, the minigene comprises, consists of, or consists essentially of a nucleotide sequence corresponding to the amino acid sequence of SEQ ID NO:76, SEQ ID NO:77, SEQ ID NO:78, SEQ ID NO:79, or SEQ ID NO:80.

In some embodiments, the minigene comprises a nucleotide sequence corresponding to an amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, or any value derivable therein, with DAEFRHDSGYEVHHQKLPFFAEDVGSNKGAIIGLMVGGVVIATVIVITLVMLKK (SEQ ID NO:81), DAEFRHDSGYEVHHQKLPFFAEDVGSNKGAIIGLMVGGVVIATVIVITLVMLKKK (SEQ ID NO:82), DAEFRHDSGYEVHHQKLPFFAEDVGSNKGAIIGLMVGGVVIATVIVITLVMAKKK (SEQ ID NO:83), DAEFRHDSGYEVHHQKLPFFAEDVGSNKGAIIGLMVGGVVIATVIVITLVMLRKK (SEQ ID NO:84), DAEFRHDSGYEVHHQKLPFFAEDVGSNKGAIIGLMVGGVVIATVIVITLVMARKK (SEQ ID NO:85), or a fragment or functional derivative thereof, wherein the bolded text corresponds to an Aβ peptide variant amino acid sequence, the underlined text corresponds to a transmembrane domain amino acid sequence, and the unmodified text corresponds to a cytosolic amino acid sequence. In some embodiments, the minigene comprises, consists of, or consists essentially of a nucleotide sequence corresponding to the amino acid sequence of SEQ ID NO:81, SEQ ID NO:82, SEQ ID NO:83, SEQ ID NO:84, or SEQ ID NO:85.

In some embodiments, the minigene comprises a nucleotide sequence corresponding to an amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, or any value derivable therein, with DAEFRHDSGYEVHHQKLVFFAEDVGSNKGAIIGLMVGGVVIATVIVITLVMLKK (SEQ ID NO:13), DAEFRHDSGYEVHHQKLVFFAEDVGSNKGAIIGLMVGGVVIATVIVITLVMLKKK (SEQ ID NO:14), DAEFRHDSGYEVHHQKLVFFAEDVGSNKGAIIGLMVGGVVIATVIVITLVMAKK K (SEQ ID NO:15), DAEFRHDSGYEVHHQKLVFFAEDVGSNKGAIIGLMVGGVVIATVIVITLVMLRKK (SEQ ID NO:16), DAEFRHDSGYEVHHQKLVFFAEDVGSNKGAIIGLMVGGVVIATVIVITLVMARK K (SEQ ID NO:17), or a fragment or functional derivative thereof, wherein the bolded text corresponds to the wild-type Aβ42 peptide amino acid sequence, the underlined text corresponds to a transmembrane domain amino acid sequence, and the unmodified text corresponds to a cytosolic amino acid sequence. In some embodiments, the minigene comprises, consists of, or consists essentially of a nucleotide sequence corresponding to the amino acid sequence of SEQ ID NO:13, SEQ ID NO:14, SEQ ID NO:15, SEQ ID NO:16, or SEQ ID NO:17.

In some embodiments, the transmembrane domain comprises a nucleotide sequence corresponding to the 13 C-terminal amino acids of Aβ peptide variants and an additional 10 amino acids. Thus, one of skill in the art would recognize that the amino acid sequence of Aβ peptide variants comprises part of, or overlaps with, the amino acid sequence of the transmembrane domain. In some embodiments, the amino acid sequence of the transmembrane portion of the minigene encoding an Aβ peptide variant comprises AIIGLMVGGVVIATVIVITLVML (SEQ ID NO:9), AIIGLMVGGVVIATVIVITLVMA (SEQ ID NO:18), AIIGPMVGGVVIATVIVITLVML (SEQ ID NO:86), or AIIGPMVGGVVIATVIVITLVMA (SEQ ID NO:87), where the bolded text represents the portion of the transmembrane domain amino acid sequence that overlaps with the amino acid sequence of Aβ peptide variants. In some embodiments, the minigene comprises a transmembrane sequence corresponding to an amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, or any value derivable therein, with either of SEQ ID NO:9, SEQ ID NO:18, SEQ ID NO:86, or SEQ ID NO:87.

In some embodiments, the cytosolic sequence comprises a nucleotide sequence corresponding to an amino acid sequence for membrane anchoring, promotion of gamma-secretase cleavage, and/or extracellular release of Aβ peptide. In some embodiments, the cytosolic sequence comprises a nucleotide sequence corresponding to an amino acid sequence comprising two lysine residues (e.g., KK). In some embodiments, the cytosolic sequence comprises a nucleotide sequence corresponding to an amino acid sequence comprising three lysine residues (e.g., KKK). In some embodiments in which the cytosolic amino acid sequence comprises three lysine residues, the first, or 5′, lysine may be substituted with an arginine residue (e.g., RKK).

In some embodiments, the signal peptide sequence comprises an amino acid sequence described in L. Kober et al., Biotechnology and Bioengineering 110(4):1164-1173 (2013), specifically incorporated by reference herein in its entirety. Any signal sequence sufficient to ensure trafficking of an Aβ-containing pro-peptide to the cell surface, which can then be cleaved off by endogenous proteases to expose the Aβ peptide N-terminus, is contemplated for use in the polynucleotide constructs of the present disclosure.

As a non-limiting example, in some embodiments, the signal peptide sequence comprises a nucleotide sequence corresponding to an amino acid sequence comprising a Gaussia luciferase signal peptide, and the Gaussia luciferase signal peptide may be codon-optimized for mammalian gene expression, as described in B. A. Tannous et al., Molecular Therapy 11(3):435-443 (2005), and S. Knappskog et al., J. of Biotechnology 128:705-715 (2007), specifically incorporated by reference herein in their entirety. In some embodiments, an amino acid sequence of a Gaussia luciferase signal peptide comprises MGVKVLFALICIAVAEA (SEQ ID NO:19). A corresponding nucleotide sequence encoding the Gaussia luciferase signal peptide of SEQ ID NO:19 may comprise ATGGGCGTGAAGGTCCTGTTCGCCCTGATTTGCATCGCCGTCGCAGAGGCA (SEQ ID NO: 20).

As a second non-limiting example, in some embodiments, the signal peptide sequence comprises a nucleotide sequence corresponding to an amino acid sequence comprising a mouse immunoglobulin heavy chain (MoIgH) signal peptide, and the MoIgH signal peptide may be codon-optimized for mammalian gene expression, as described in B. A. Tannous et al., Molecular Therapy 11(3):435-443 (2005), and S. Knappskog et al., J. of Biotechnology 128:705-715 (2007), specifically incorporated by reference herein in their entirety. In some embodiments, an amino acid sequence of a MoIgH signal peptide comprises MGWSCIILFLVATATGVHS (SEQ ID NO:21). A corresponding nucleotide sequence encoding the MoIgH signal peptide of SEQ ID NO:21 may comprise ATGGGATGGAGCTGTATCATCCTCTTCTTGGTAGCAACAGCTACAGGTAAGGGG CTCACAGTAGCAGGCTTGAGGTCTGGACATATATATGGGTGACAATGACAT CCACTTTGCCTTTCTCTCCACAGGTGTCCACTCC (SEQ ID NO: 22), wherein the bolded portion of SEQ ID NO:22 corresponds to an intronic non-coding sequence of the MoIgH signal peptide.

In some embodiments, the minigene comprises a signal peptide sequence corresponding to an amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, or any value derivable therein, with either of SEQ ID NO:19 or SEQ ID NO:21.

In some embodiments, the polynucleotide is comprised in a vector. In some embodiments, the vector stably expresses a wild-type or variant Aβ peptide, or a fragment or functional derivative thereof, at the cell membrane where release of the Aβ peptide into the extracellular space is regulated by endogenous γ-secretase.

A. Proteinaceous Compositions

As used herein, a “proteinaceous molecule,” “proteinaceous composition,” “proteinaceous compound,” “proteinaceous chain” or “proteinaceous material” generally refers, but is not limited to, a protein of greater than about 200 amino acids or the full length endogenous sequence translated from a gene; a polypeptide of greater than about 100 amino acids; and/or a peptide of from about 3 to about 100 amino acids. All the “proteinaceous” terms described above may be used interchangeably herein.

As used herein, a “protein,” “peptide,” or “polypeptide” refers to a molecule comprising at least three amino acid residues. As used herein, the term “endogenous” refers to the version of a molecule that occurs naturally in an organism. In some embodiments, the endogenous peptide comprises a wild-type peptide. In some embodiments, the endogenous peptide comprises amino acid alterations from a wild-type peptide. In some embodiments, wild-type versions of a protein or peptide are employed, however, in many embodiments of the disclosure, a modified protein or peptide is employed. The terms described above may be used interchangeably. A “modified protein” or “modified polypeptide” or a “variant” refers to a protein or peptide whose chemical structure, particularly its amino acid sequence, is altered with respect to the wild-type protein or peptide. In some embodiments, a modified/variant protein or peptide has at least one modified activity or function (recognizing that proteins or polypeptides may have multiple activities or functions). It is specifically contemplated that a modified/variant protein or peptide may be altered with respect to one activity or function yet retain a wild-type activity or function in other respects.

Where a protein is specifically mentioned herein, it is in general a reference to a native (endogenous) or recombinant (modified or variant) protein or peptide. The protein may be isolated directly from the organism of which it is native, produced by recombinant DNA/exogenous expression methods, or produced by solid-phase peptide synthesis (SPPS) or other in vitro methods. Various automatic synthesizers are commercially available and can be used in accordance with known protocols. See, for example, Stewart and Young, (1984); Tam et al., (1983); Merrifield, (1986); and Barany and Merrifield (1979), each specifically incorporated by reference herein in its entirety. Alternatively, recombinant DNA technology may be employed wherein a nucleotide sequence that encodes a peptide or polypeptide is inserted into an expression vector, transformed or transfected into an appropriate host cell and cultivated under conditions suitable for expression. In particular embodiments, there are isolated nucleic acid segments and recombinant vectors incorporating nucleic acid sequences that encode a peptide. The term “recombinant” may be used in conjunction with a peptide or the name of a specific peptide, and this generally refers to a peptide produced from a nucleic acid molecule that has been manipulated in vitro or that is a replication product of such a molecule.

In certain embodiments, the size of the at least one proteinaceous molecule may comprise, but is not limited to, about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, about 100, about 110, about 120, about 130, about 140, about 150, about 160, about 170, about 180, about 190, about 200, about 210, about 220, about 230, about 240, about 250, about 275, about 300, about 325, about 350, about 375, about 400, about 425, about 450, about 475, about 500, about 525, about 550, about 575, about 600, about 625, about 650, about 675, about 700, about 725, about 750, about 775, about 800, about 825, about 850, about 875, about 900, about 925, about 950, about 975, about 1000, about 1100, about 1200, about 1300, about 1400, about 1500, about 1750, about 2000, about 2250, about 2500 or greater amino molecule residues, and any range derivable therein, or derivative of a corresponding amino sequence described or referenced herein.

The peptides described herein may be of a fixed length of at least, at most, or exactly 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107,108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 300, 400, 500, 550, 1000 or more amino acids (or any derivable range therein).

In certain embodiments the proteinaceous composition comprises at least one protein, polypeptide or peptide. It is contemplated that virtually any protein, polypeptide, or peptide containing component described herein may be used in the compositions and methods disclosed herein. In further embodiments the proteinaceous composition comprises a biocompatible protein, polypeptide, or peptide. As used herein, the term “biocompatible” refers to a substance which produces no significant untoward effects when applied to, or administered to, a given organism according to the methods and amounts described herein. Such untoward or undesirable effects are those such as significant toxicity or adverse immunological reactions. In preferred embodiments, biocompatible protein-, polypeptide-, or peptide-containing compositions will generally be mammalian proteins or peptides or synthetic proteins or peptides each essentially free from toxins, pathogens and harmful immunogens.

Proteinaceous compositions may be made by any technique known to those of skill in the art, including the expression of proteins, polypeptides, or peptides through standard molecular biological techniques, the isolation of proteinaceous compounds from natural sources, or the chemical synthesis of proteinaceous materials. The nucleotide and protein, polypeptide, and peptide sequences for various genes have been previously disclosed, and may be found at computerized databases known to those of ordinary skill in the art. One such database is the National Center for Biotechnology Information's Genbank and GenPept databases (http://www.ncbi.nlm.nih.gov/). The coding regions for these known genes may be amplified and/or expressed using the techniques disclosed herein or as would be known to those of ordinary skill in the art. Alternatively, various commercial preparations of proteins, polypeptides, and peptides are known to those of skill in the art.

In certain embodiments a proteinaceous compound may be purified. Generally, “purified” will refer to a specific or protein, polypeptide, or peptide composition that has been subjected to fractionation to remove various other proteins, polypeptides, or peptides, and which composition substantially retains its activity, as may be assessed, for example, by the protein assays, as would be known to one of ordinary skill in the art for the specific or desired protein, polypeptide, or peptide.

Proteins and peptides suitable for use in this invention may be autologous proteins or peptides, although the invention is clearly not limited to the use of such autologous proteins. As used herein, the term “autologous protein, polypeptide, or peptide” refers to a protein, polypeptide or peptide which is derived or obtained from an organism. Organisms that may be used include, but are not limited to, a bovine, a reptilian, an amphibian, a piscine, a rodent, an avian, a canine, a feline, a fungal, a plant, or a prokaryotic organism, with a selected animal or human subject being preferred. The “autologous protein, polypeptide or peptide” may then be used as a component of a composition intended for application to the selected animal or human subject.

In that the compositions of the present disclosure are particularly suitable for use in treating or preventing neurodegenerative diseases, preferred proteins, including wild-type or variant Aβ peptides, are contemplated.

To select other proteins, polypeptides, peptides, and the like for use in the methods and compositions of the present disclosure, one would preferably select a proteinaceous material that possesses one or more of the following characteristics: it forms a solution with a high percentage of proteinaceous material solubilized; it possesses a high viscosity (i.e. about 40 to about 100 poise); it has the correct molecular charge to bind a dye if it is a non-covalent mixture (i.e. anionic protein and cationic dye, or cationic protein and anionic dye); it has the correct amino acids present to form covalent cross-links (i.e. one or more tyrosines, histidines, tryptophans and/or methionines); and/or it is biocompatible (i.e. from mammalian origin for mammals, preferably from human origin for humans, from canine origin for canines, etc.; it is autologous; it is non-allergenic, and/or it is non-immunogenic).

It is contemplated that in compositions of the disclosure, there is between about 0.001 mg and about 10 mg of total polypeptide, peptide, and/or protein per ml. The concentration of protein in a composition can be about, at least about or at most about 0.001, 0.010, 0.050, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0 mg/ml or more (or any range derivable therein).

B. Biological Functional Equivalents

As modifications and/or changes may be made in the structure of polynucleotides and/or proteins according to the present invention, while obtaining molecules having similar or improved characteristics, such biologically functional equivalents are also encompassed within the present invention.

1. Modified Polynucleotides and Peptides

The biological functional equivalent may comprise a polynucleotide that has been engineered to contain distinct sequences while at the same time retaining the capacity to encode the “wild-type” or standard protein or peptide or “variant” protein or peptide. This can be accomplished to the degeneracy of the genetic code, i.e., the presence of multiple codons, which encode for the same amino acids. The term “functionally equivalent codon” is used herein to refer to codons that encode the same amino acid, such as the six different codons for arginine. Also considered are “neutral substitutions” or “neutral mutations” which refers to a change in the codon or codons that encode biologically equivalent amino acids. In one example, one of skill in the art may wish to introduce a restriction enzyme recognition sequence into a polynucleotide while not disturbing the ability of that polynucleotide to encode a protein.

In terms of functional equivalents, it is well understood by the skilled artisan that, inherent in the definition of a “biologically functional equivalent” protein and/or polynucleotide, is the concept that there is a limit to the number of changes that may be made within a defined portion of the molecule while retaining a molecule with an acceptable level of equivalent biological activity. Biologically functional equivalents are thus defined herein as those proteins (and polynucleotides) having substitutions or mutations in selected amino acids (or codons) that retain the ability to interfere with endogenous Aβ peptide aggregation and/or to promote disaggregation of endogenous Aβ peptide fibrils or other endogenous Aβ peptide multimer structures and/or proteins (and polynucleotides) having substitutions or mutations in selected amino acids (or codons) that that do not self-aggregate, such that toxicity and/or aggregation of endogenous Aβ peptide is diminished, for example. Biologically functional equivalents may also include those proteins (and polynucleotides) having substitutions or mutations in selected amino acids (or codons) that retain the ability to promote endogenous Aβ peptide aggregation and/or aggregation of endogenous Aβ peptide fibrils or other endogenous Aβ peptide multimer structures and/or proteins (and polynucleotides) having substitutions or mutations in selected amino acids (or codons) that that do self-aggregate, for example.

In general, the shorter the length of the molecule, the fewer changes that can be made within the molecule while retaining function. Longer domains may have an intermediate number of changes. The full-length protein will have the most tolerance for a larger number of changes. However, it must be appreciated that certain molecules or domains that are highly dependent upon their structure may tolerate little or no modification.

In one example, a polynucleotide may be (and encode) a biological functional equivalent with more significant changes. Certain amino acids may be substituted for other amino acids in a protein structure without appreciable loss of interactive binding capacity with structures such as, for example, antigen-binding regions of antibodies, binding sites on substrate molecules, receptors, and such like.

Substitutional variants typically contain the exchange of one amino acid for another at one or more sites within the protein, and may be designed to modulate one or more properties of the polypeptide, with or without the loss of other functions or properties. Substitutions may be conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine. So-called “conservative” changes do not disrupt the biological activity of the protein, as the structural change is not one that impinges of the protein's ability to carry out its designed function. It is thus contemplated by the inventors that various changes may be made in the sequence of genes and proteins disclosed herein, while still fulfilling the goals of the present invention. Alternatively, substitutions may be non-conservative such that a function or activity of the polypeptide is affected. Non-conservative changes typically involve substituting a residue with one that is chemically dissimilar, such as a polar or charged amino acid for a nonpolar or uncharged amino acid, and vice versa. Non-conservative substitutions may involve the exchange of a member of one of the amino acid classes for a member from another class.

In other embodiments, alteration of the function of a polypeptide is intended by introducing one or more substitutions. For example, certain amino acids may be substituted for other amino acids in a protein structure without appreciable loss of interactive binding capacity. Structures such as, for example, an enzymatic catalytic domain or interaction components may have amino acid substituted to maintain such function. Since it is the interactive capacity and nature of a protein that defines that protein's biological functional activity, certain amino acid substitutions can be made in a protein sequence, and in its underlying DNA coding sequence, and nevertheless produce a protein with like properties. It is thus contemplated by the inventors that various changes may be made in the DNA sequences of genes without appreciable loss of their biological utility or activity.

Deletion variants typically lack one or more residues of the native or wild-type protein. Individual residues can be deleted or a number of contiguous amino acids can be deleted. A stop codon may be introduced (by substitution or insertion) into an encoding nucleic acid sequence to generate a truncated protein. For example, it is contemplated that peptides may be mutated by truncation, or deletion of a number of contiguous amino acids, rendering them shorter than their corresponding endogenous form.

Insertional mutants typically involve the addition of amino acid residues at a non-terminal point in the polypeptide. This may include the insertion of one or more amino acid residues. Terminal additions may also be generated and can include fusion proteins which are multimers or concatemers of one or more peptides or polypeptides described or referenced herein. For example, it is contemplated that peptides might be altered by fusing or conjugating a heterologous protein or polypeptide sequence with a particular function (e.g., for targeting or localization, for enhanced activity, for purification purposes, etc.).

Additionally, the polypeptides of the disclosure may be chemically modified. Glycosylation of the polypeptides can be altered, for example, by modifying one or more sites of glycosylation within the polypeptide sequence to increase the affinity of the polypeptide for antigen (U.S. Pat. Nos. 5,714,350 and 6,350,861).

It also will be understood that amino acid and nucleic acid sequences may include additional residues, such as additional N- or C-terminal amino acids, or 5′ or 3′ sequences, respectively, and yet still be essentially as set forth in one of the sequences disclosed herein, so long as the sequence meets the criteria set forth above, including the maintenance of biological protein activity where protein expression is concerned. The addition of terminal sequences particularly applies to nucleic acid sequences that may, for example, include various non-coding sequences flanking either of the 5′ or 3′ portions of the coding region.

Amino acid sequence variants of the disclosure can be substitutional, insertional, or deletion variants, for example. It is contemplated that a region or fragment of a polypeptide of the disclosure may have an amino acid sequence that has, has at least, or has at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107,108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200 or more amino acid substitutions, contiguous amino acid additions, or contiguous amino acid deletions with respect to any of SEQ ID NOs:1-19, SEQ ID NO:21, 23-39, and 65-87. Alternatively, a region or fragment of a polypeptide of the disclosure may have an amino acid sequence that comprises or consists of an amino acid sequence that is, is at least, or is at most 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100% (or any range derivable therein) identical to any of SEQ ID NOs:1-19, SEQ ID NO:21, 23-39, and 65-87.

Moreover, in some embodiments, a region or fragment comprises an amino acid region of 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500 or more contiguous amino acids starting at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500 in any of SEQ ID NOs:1-19, SEQ ID NO:21, 23-39, and 65-87 (where position 1 is at the N-terminus of the SEQ ID NO). The polypeptides of the disclosure may include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 or more variant amino acids or be at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% similar, identical, or homologous with at least, or at most 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106,107, 108, 109, 110, 111, 112, 113, 114, 115, 116, 117, 118, 119, 120, 121,122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 300, 400, 500, 550, 600, or more contiguous amino acids, or any range derivable therein, of any of SEQ ID NOs:1-19, SEQ ID NO:21, 23-39, and 65-87.

The polypeptides of the disclosure may include at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107,108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, or 615 substitutions (or any range derivable therein).

The substitution may be at amino acid position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110,111, 112, 113, 114, 115, 116, 117, 118, 119, 120,121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, or 650 of any of SEQ ID NOs:1-19, SEQ ID NO:21, 23-39, and 65-87 (or any derivable range therein).

Amino acid substitutions are generally based on the relative similarity of the amino acid side-chain substituents, for example, their hydrophobicity, hydrophilicity, charge, size, and/or the like. An analysis of the size, shape and/or type of the amino acid side-chain substituents reveals that arginine, lysine and/or histidine are all positively charged residues; that alanine, glycine and/or serine are all a similar size; and/or that phenylalanine, tryptophan and/or tyrosine all have a generally similar shape. Therefore, based upon these considerations, arginine, lysine and/or histidine; alanine, glycine and/or serine; and/or phenylalanine, tryptophan and/or tyrosine; are defined herein as biologically functional equivalents.

In making such changes to produce biologically functional equivalents, the hydropathic index of amino acids may be considered. The hydropathy profile of a protein is calculated by assigning each amino acid a numerical value (“hydropathy index”) and then repetitively averaging these values along the peptide chain. Each amino acid has been assigned a value based on its hydrophobicity and charge characteristics. They are: isoleucine (+4.5); valine (+4.2); leucine (+3.8); phenylalanine (+2.8); cysteine/cysteine (+2.5); methionine (+1.9); alanine (+1.8); glycine (−0.4); threonine (−0.7); serine (−0.8); tryptophan (−0.9); tyrosine (−1.3); proline (1.6); histidine (−3.2); glutamate (−3.5); glutamine (−3.5); aspartate (−3.5); asparagine (−3.5); lysine (−3.9); and arginine (−4.5). The importance of the hydropathic amino acid index in conferring interactive biologic function on a protein is generally understood in the art (Kyte and Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like. It is also known that certain amino acids may be substituted for other amino acids having a similar hydropathy index or score, and still retain a similar biological activity. In making changes based upon the hydropathy index, in certain embodiments, the substitution of amino acids whose hydropathy indices are within ±2 is included. In some aspects of the disclosure, those that are within ±1 are included, and in other aspects of the disclosure, those within ±0.5 are included.

It also is understood in the art that the substitution of like amino acids can be made effectively on the basis of hydrophilicity. U.S. Pat. No. 4,554,101, specifically incorporated by reference herein in its entirety, states that the greatest local average hydrophilicity of a protein, as governed by the hydrophilicity of its adjacent amino acids, correlates with a biological property of the protein. In certain embodiments, the greatest local average hydrophilicity of a protein, as governed by the hydrophilicity of its adjacent amino acids, correlates with a biological property of the protein. The following hydrophilicity values have been assigned to these amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); and tryptophan (−3.4). In making changes based upon similar hydrophilicity values, in certain embodiments, the substitution of amino acids whose hydrophilicity values are within ±2 are included, in other embodiments, those which are within ±1 are included, and in still other embodiments, those within ±0.5 are included. In some instances, one may also identify epitopes from primary amino acid sequences based on hydrophilicity. It is understood that an amino acid can be substituted for another having a similar hydrophilicity value and still produce a biologically equivalent and immunologically equivalent protein.

Additionally, one skilled in the art can review structure-function studies identifying residues in similar polypeptides or proteins that are important for activity or structure. In view of such a comparison, one can predict the importance of amino acid residues in a protein that correspond to amino acid residues important for activity or structure in similar proteins. One skilled in the art may opt for chemically similar amino acid substitutions for such predicted important amino acid residues.

One skilled in the art can also analyze the three-dimensional structure and amino acid sequence in relation to that structure in similar proteins or polypeptides. In view of such information, one skilled in the art may predict the alignment of amino acid residues with respect to its three-dimensional structure. One skilled in the art may choose not to make changes to amino acid residues predicted to be on the surface of the protein, since such residues may be involved in important interactions with other molecules. Moreover, one skilled in the art may generate test variants containing a single amino acid substitution at each desired amino acid residue. These variants can then be screened using standard assays for binding and/or activity, thus yielding information gathered from such routine experiments, which may allow one skilled in the art to determine the amino acid positions where further substitutions should be avoided either alone or in combination with other mutations. Various tools available to determine secondary structure can be found on the World Wide Web at expasy.org/proteomics/protein_structure.

In some embodiments of the disclosure, amino acid substitutions are made that: (1) reduce susceptibility to proteolysis, (2) reduce susceptibility to oxidation, (3) alter binding affinity for forming protein complexes, (4) alter ligand binding affinities, and/or (5) confer or modify other physicochemical or functional properties on such polypeptides. For example, single or multiple amino acid substitutions (in certain embodiments, conservative amino acid substitutions) may be made in the naturally occurring sequence. Substitutions can be made in that portion of the protein that lies outside the domain(s) forming intermolecular contacts. In such embodiments, conservative amino acid substitutions can be used that do not substantially change the structural characteristics of the protein or polypeptide (e.g., one or more replacement amino acids that do not disrupt the primary, secondary, or tertiary structure that characterizes the native protein).

As outlined above, amino acid substitutions generally are based on the relative similarity of the amino acid side-chain substituents, for example, their hydrophobicity, hydrophilicity, charge, size, and the like. Exemplary substitutions that take into consideration the various foregoing characteristics are well known and include: arginine and lysine; glutamate and aspartate; serine and threonine; glutamine and asparagine; and valine, leucine and isoleucine.

2. Altered Amino Acids

As used herein, an “amino molecule” refers to any amino acid, amino acid derivative, or amino acid mimic as would be known to one of ordinary skill in the art. In certain embodiments, the residues of the proteinaceous molecule are sequential, without any non-amino molecule interrupting the sequence of amino molecule residues. In other embodiments, the sequence may comprise one or more non-amino molecule moieties. In particular embodiments, the sequence of residues of the proteinaceous molecule may be interrupted by one or more non-amino molecule moieties. Peptides and polypeptides include the twenty “natural” amino acids, and post-translational modifications thereof. However, in vitro peptide synthesis permits the use of modified and/or unusual amino acids.

Accordingly, the term “proteinaceous composition” encompasses amino molecule sequences comprising at least one of the 20 common amino acids in naturally synthesized proteins, or at least one modified or unusual amino acid, including but not limited to those shown in the Table below.

Modified and Unusual Amino Acids

| Abbr. | Amino Acid | Abbr. | Amino Acid |
|---|---|---|---|
| Aad | 2-Aminoadipic acid | EtAsn | N-Ethylasparagine |
| Baad | 3-Aminoadipic acid | Hyl | Hydroxylysine |
| Bala | β-alanine, | AHyl | allo-Hydroxylysine |
| | β-Amino-propionic acid | | |
| Abu | 2-Aminobutyric acid | 3Hyp | 3-Hydroxyproline |
| 4Abu | 4-Aminobutyric acid, | 4Hyp | 4-Hydroxyproline |
| | piperidinic acid | | |
| Acp | 6-Aminocaproic acid | Ide | Isodesmosine |
| Ahe | 2-Aminoheptanoic acid | AIle | allo-Isoleucine |
| Aib | 2-Aminoisobutyric acid | MeGly | N-Methylglycine, |
| | | | sarcosine |
| Baib | 3-Aminoisobutyric acid | MeIle | N-Methylisoleucine |
| Apm | 2-Aminopimelic acid | MeLys | 6-N-Methyllysine |
| Dbu | 2,4-Diaminobutyric acid | MeVal | N-Methylvaline |
| Des | Desmosine | Nva | Norvaline |
| Dpm | 2,2′-Diaminopimelic acid | Nle | Norleucine |
| Dpr | 2,3-Diaminopropionic acid | Orn | Ornithine |
| EtGly | N-Ethylglycine | | |

3. Mimetics

In addition to the biological functional equivalents discussed above, the present inventors also contemplate that structurally similar compounds may be formulated to mimic the key portions of peptide or polypeptides of the present invention. Such compounds, which may be termed peptidomimetics, may be used in the same manner as the peptides of the invention and, hence, also are functional equivalents.

Certain mimetics that mimic elements of protein secondary and tertiary structure are described in Johnson et al. (1993). The underlying rationale behind the use of peptide mimetics is that the peptide backbone of proteins exists chiefly to orient amino acid side chains in such a way as to facilitate molecular interactions, such as those of antibody and/or antigen. A peptide mimetic is thus designed to permit molecular interactions similar to the natural molecule.

Some successful applications of the peptide mimetic concept have focused on mimetics of β-turns within proteins, which are known to be highly antigenic. Likely β-turn structure within a polypeptide can be predicted by computer-based algorithms, as discussed herein. Once the component amino acids of the turn are determined, mimetics can be constructed to achieve a similar spatial orientation of the essential elements of the amino acid side chains.

Other approaches have focused on the use of small, multidisulfide-containing proteins as attractive structural templates for producing biologically active conformations that mimic the binding sites of large proteins. Vita et al. (1998). A structural motif that appears to be evolutionarily conserved in certain toxins is small (30-40 amino acids), stable, and high permissive for mutation. This motif is composed of a beta sheet and an alpha helix bridged in the interior core by three disulfides.

Beta II turns have been mimicked successfully using cyclic L-pentapeptides and those with D-amino acids. Weisshoff et al. (1999). Also, Johannesson et al. (1999) report on bicyclic tripeptides with reverse turn inducing properties.

Methods for generating specific structures have been disclosed in the art. For example, alpha-helix mimetics are disclosed in U.S. Pat. Nos. 5,446,128; 5,710,245; 5,840,833; and 5,859,184. These structures render the peptide or protein more thermally stable, also increase resistance to proteolytic degradation. Six, seven, eleven, twelve, thirteen and fourteen membered ring structures are disclosed.

Methods for generating conformationally restricted beta turns and beta bulges are described, for example, in U.S. Pat. Nos. 5,440,013; 5,618,914; and 5,670,155. Beta-turns permit changed side substituents without having changes in corresponding backbone conformation, and have appropriate termini for incorporation into peptides by standard synthesis procedures. Other types of mimetic turns include reverse and gamma turns. Reverse turn mimetics are disclosed in U.S. Pat. Nos. 5,475,085 and 5,929,237, and gamma turn mimetics are described in U.S. Pat. Nos. 5,672,681 and 5,674,976.

C. Exogenous Delivery of Peptides

In certain embodiments, modifications may be made to the proteinaceous compositions and peptide variants disclosed herein to improve exogenous delivery of the peptides to cells.

In certain embodiments, vectors could be constructed to comprise exogenous nucleic acid sequences to allow cells to express the proteinaceous compositions and peptide variants disclosed herein. Details of components of these vectors and delivery methods are disclosed below.

In some embodiments, administration of the compositions disclosed herein can cause cells to contain one or more genetic alterations by genetic engineering of the cells. A cell is said to be “genetically altered”, “genetically modified” or “transgenic” when an exogenous nucleic acid or polynucleotide has been transferred into the cell by any suitable means of artificial manipulation, or where the cell is a progeny of the originally altered cell that has inherited the polynucleotide.

In various embodiments a DNA construct or vector encoding peptide sequences of wildtype or variant Aβ peptides or fragments or functional derivatives thereof disclosed herein are provided. Genetic modification may also be introduced to cells. These modifications include, for example, transduction of cells with a vector encoding a wild-type Aβ peptide or an Aβ peptide variant or a fragment or functional derivative thereof for the generation of cells which express a wild-type Aβ peptide or an Aβ peptide variant or a fragment or functional derivative thereof. Viral vectors encoding peptide sequences of a wild-type Aβ peptide or an Aβ peptide variant or a fragment or functional derivative thereof disclosed herein allow continuous gene expression and Aβ peptide release.

Cells according to the present disclosure include any cell into which the proteinaceous compositions and peptide variants disclosed herein and/or DNA constructs or vectors constructed to comprise exogenous nucleic acid sequences to allow cells to express the proteinaceous compositions and peptide variants disclosed herein can be introduced and expressed as described herein. It is to be understood that the basic concepts of the present disclosure described herein are not limited by cell type. Cells according to the present disclosure include eukaryotic cells, mammalian cells, animal cells, human cells and the like. Further, cells include any in which it would be beneficial or desirable to regulate production of a functional protein.

1. Peptide Modifications to Improve Exogenous Delivery

In some embodiments, wildtype or variant Aβ peptides disclosed herein may be modified to enhance their uptake or absorption by cells. For example, in some embodiments, a cell-penetrating peptide (CPP) is attached to wildtype or variant Aβ peptides to produce a cell-permeable and/or brain-penetrant inhibitor of Aβ oligomer and fibril formation. CPPs are short peptides that facilitate cellular intake and uptake of molecules ranging from nanosize particles to small chemical compounds to large fragments of DNA. The cargo is associated with the peptides either through chemical linkage via covalent bonds or through non-covalent interactions. CPPs typically have an amino acid composition that either contains a high relative abundance of positively charged amino acids such as lysine or arginine or has sequences that contain an alternating pattern of polar, charged amino acids and non-polar, hydrophobic amino acids. These two types of structures are referred to as polycationic or amphipathic, respectively. A third class of CPPs are the hydrophobic peptides, containing only apolar residues with low net charge or hydrophobic amino acid groups that are crucial for cellular uptake.

In some embodiments, the cell-penetrating peptide is retro-inverted version of the HIV protein transduction domain “TAT”; Poly-arginine (e.g., R8-R12); Polyamines; MAP; MTS; MPG; Penetratin; Pep-1; Transportan; or VP22. In some embodiments, the cell-penetrating peptide is retro-inverted version of the HIV protein transduction domain “TAT.” In some embodiments, the cell-penetrating peptide is a poly-arginine. In some embodiments, the cell-penetrating peptide is a polyamine. In some embodiments, attaching a cell-penetrating peptide to wildtype or variant Aβ peptides renders the wildtype or variant Aβ peptides cell permeant without vectorization.

Transactivating transcriptional activator (TAT), from human immunodeficiency virus 1 (HIV-1) allows penetration of the impermeable phospholipid bilayer of cell membranes and the crossing of biological barriers. See M. Green et al., Biochim Biophys Acta 1414:127-139 (1998), specifically incorporated herein by reference in its entirety). Following secretion from HIV-infected cells, TAT translocates into neighboring cells to modify gene transcription and spread the disease. The first report that demonstrated that a Tat-derived peptide can deliver a large protein into different cell types and mammalian organs was published in 1994, which demonstrated chemical cross-linking and identification of a 36-amino acid region of HIV-1 that was able to promote the uptake of β-galactosidase as a chimera into living cells. See S. Fawell et al., Proc Natl Acad Sci USA 91:664-668 (1994). This HIV TAT protein transduction domain (PTD) contains a cluster of basic amino acid residues and a sequence assumed to adopt an α-helical configuration. Countless studies aimed to delineate whether shorter domains of this TAT peptide would be sufficient for cell internalization. The main determinant required for translocation was identified as the cluster of basic amino acids, while the putative α-helix domain appeared dispensable, although peptides with an α-helical region can more efficiently enter cells. The truncated polycationic peptide GRKKRRQRRR that includes RNA binding and nuclear localization signal (NLS) motifs was identified to be adequate for effective translocation into cells and tissues and is comprised in TAT. See E. Vives E et al., J Biol Chem 272:16010-16017 (1997). Thus, in some embodiments, wildtype or variant Aβ peptides are attached to a TAT sequence. A TAT sequence can comprise peptides having an amino acid sequence comprising GRKKRRQRRR (SEQ ID NO:23), YGRKKRRQRRR (SEQ ID NO:24), GRKKRRQRRRPQ (SEQ ID NO:25), an amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, or any value derivable therein, with any of SEQ ID NOs:23-25, or any fragment or derivative thereof.

Oligo- and poly-arginines are structurally the simplest CPPs with Arg as the only building block and can be readily prepared. Poly-arginines adopt random coil conformation in aqueous solution or when associated with phospholipid membranes due to the strong side chain charge repulsion and lack of hydrophobic or amphiphilic structure, and their membrane permeability mainly relies on the electrostatic interaction with lipid membranes mediated by their guanidinium charge groups. Thus, in some embodiments, wildtype or variant Aβ peptides are attached to a poly-arginine. In some embodiments, the poly-arginine comprises a polymer of L- or D-arginine containing six or more arginine amino acids. In some embodiments, the poly-arginine comprises R8-R12, corresponding to an amino acid sequence comprising RRRRRRRR-RRRRRRRRRRRR (SEQ ID NO:26) (see G. Tunnemann et al., J. Pept. Sci. 14:469-476 (2008), specifically incorporated herein by reference in its entirety).

Polyamines, for example, putrescine, spermidine, and spermine, have also been shown to increase the permeability of proteins at the blood-nerve and blood-brain barriers. The polyamine transporter may be responsible for the transport of polyamine-modified proteins. See, e.g., J. F. Poduslo & G. L. Curran, J. Neurochem 66:5705-5709 (1996); J. F. Poduslo & G. L. Curran, J. Neurochem 67:734-741 (1996); and J. F. Poduslo et al., J. Neurobio 39(3):371-82 (1999), specifically incorporated herein by reference in their entirety. Thus, systemic administration of polyamine-modified wildtype or variant Aβ peptides can be an efficient approach to deliver these therapeutic agents into the CNS for the treatment of a variety of neurological diseases, including AD, and in some embodiments, wildtype or variant Aβ peptides are covalently attached to a polyamine. In some embodiments, the polyamine is putrescine. In some embodiments, the polyamine is spermidine. In some embodiments, the polyamine is spermine.

Other cell-penetrating peptides contemplated for attachment to wildtype or variant Aβ peptides disclosed herein include, but are not limited to, the following cell-penetrating peptides: MAP, corresponding to an amino acid sequence comprising KLALKLALKALKAALKLA (SEQ ID NO:27) (see J. Oehlke et al., Cell 58:215-223 (1989), specifically incorporated herein by reference in its entirety); MTS, corresponding to an amino acid sequence comprising AAVALLPAVLLALLAP (SEQ ID NO:28) (see M. Rojas, Nat. Biotechnol. 16:370-375 (1998), specifically incorporated herein by reference in its entirety); MPG, corresponding to an amino acid sequence comprising GLAFLGFLGAAGSTMGAWSQPKKKRKV (SEQ ID NO:29) (see M. C. Morris et al., Nucleic Acids Res. 25:2730-2736 (1997), specifically incorporated herein by reference in its entirety); Penetratin, corresponding to an amino acid sequence comprising RQIKIWFQNRRMKWKK (SEQ ID NO:30) (see D. Derossi et al., J. Biol. Chem. 269:10444-10450 (1994), specifically incorporated herein by reference in its entirety); Pep-1, corresponding to an amino acid sequence comprising KETWWETWWTEWSQPKKRKV (SEQ ID NO:31) (see M. C. Morris et al., Nat. Biotechnol. 19:1173-1176 (2001), specifically incorporated herein by reference in its entirety); Transportan, corresponding to an amino acid sequence comprising GWTLNSAGYLLGKINLKALAALAKKIL (SEQ ID NO:32) (see M. Pooga et al., FASEB J. 12:67-77 (1998), specifically incorporated herein by reference in its entirety); or VP22, corresponding to an amino acid sequence comprising DAATATRGRSAASRPTQRPRAPARSASRPRRPVQ (SEQ ID NO:33) (see G. Elliott et al., Cell 88:223-233 (1997), specifically incorporated herein by reference in its entirety). Further cell-penetrating peptides contemplated for attachment to wildtype or variant Aβ peptides disclosed herein are described in, for example, A. Borrelli et al., Molecules 23:295-318 (2018), specifically incorporated herein by reference in its entirety.

In some embodiments, a cell-permeable and/or brain-penetrant Aβ peptide variant comprises an Aβ peptide variant in which the naturally occurring L-amino acids of the Aβ peptide variant is replaced with the D-enantiomers of the amino acids. See J. F. Poduslo et al., J. Neurobio 39(3):371-82 (1999). Peptides containing D-residues are more resistant to proteolytic degradation. See Robson, Nat Biotechnol 14:893-895 (1996), and Schumacher et al., Science 271:1854-1857 (1996). Furthermore, this modification may make the Aβ peptide variant less immunogenic, since L-amino acid peptides are efficiently processed for major histocompatibility complex class II-restricted presentation to T helper cells, thus generating a vigorous humoral immune response that impairs drug bioactivity, whereas D-residues peptides are significantly less antigenic. See Herve et al., J. Immunol. 156:157-163 (1997). Thus, in some embodiments, the naturally occurring L-amino acids of Aβ peptide variants disclosed herein are replaced with the D-enantiomers of the amino acids to improve cell permeability and/or brain penetrance of the Aβ peptide variants.

2. Vectors

In some embodiments, wildtype or variant Aβ peptides disclosed herein are achieved by operably linking a nucleic acid encoding the wildtype or variant Aβ peptides or portions thereof to a promoter, and incorporating the construct into an expression vector, which is taken up and expressed by cells. The vectors can be suitable for replication and, in some cases, integration in eukaryotes. Typical cloning vectors contain transcription and translation terminators, initiation sequences, and promoters useful for regulation of the expression of the desired nucleic acid sequence. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors. In general, a suitable vector contains an origin of replication functional in at least one organism, a promoter sequence, convenient restriction endonuclease sites, and one or more selectable markers (see, e.g., WO 01/96584; WO 01/29058; and U.S. Pat. No. 6,326,193). In some embodiments, a suitable vector is capable of crossing the blood-brain barrier.

In certain embodiments the expression vector may be provided to a cell in the form of a viral vector. Viral vector technology is well known in the art and is described, for example, in Sambrook et al. (2001) Molecular Cloning: A Laboratory Manual, Cold Spring Harbor Laboratory, New York), and in other virology and molecular biology manuals.

A number of viral based systems have been developed for gene transfer into mammalian cells. Viruses that are useful as vectors include, but are not limited to, retroviruses, adenoviruses, adeno-associated viruses, herpes viruses, and lentiviruses (including self-inactivating lentivirus vectors). For example, adenoviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. Thus, in some embodiments, the nucleic acid encoding an Aβ peptide variant is introduced into cells using a recombinant vector such as a viral vector including, for example, a lentivirus, a retrovirus, gamma-retroviruses, an adeno-associated virus (AAV), a herpesvirus, or an adenovirus.

In specific embodiments, the vector is an AAV vector. “AAV vector” refers to a recombinant vector derived from an adeno-associated virus serotype, such as AAV1, AAV2, AAV3, AAV4, AAV5, AAV6, AAV7, AAV8, AAV9, AAV10, AAV11, AAV12, AAV2.5, AAV-DJ, AAVrhlO.XX, AAVrh.8, AAVrh.10, AAVrh.43, AAVpi.2, AAVhu.11, AAVhu.32, AAVhu.37, PHP.eB AAV, and others. AAV vectors can have one or all wild-type AAV genes deleted, but still comprise functional inverted terminal repeat (ITR) nucleic acid sequences. Functional ITR sequences are necessary for the replication, rescue and packaging of AAV virions. The ITR sequences may be wild-type sequences or substantially identical sequences or may be altered by for example in insertion, mutation, deletion or substitution of nucleotides, as long as they remain functional. Such AAV vectors can be replicated and packaged into infectious viral particles when present in a host cell that is expressing AAV rep and cap gene products (i.e., AAV Rep and Cap proteins).

The genome of AAV is a linear, single stranded DNA molecule that is less than about 5,000 nucleotides (nt) in length. Inverted terminal repeats (ITRs) flank the unique coding nucleotide sequences for the non-structural replication (Rep) proteins and the structural (VP) proteins. The VP proteins (VP1, -2, and -3) form the capsid. The terminal 145 nts are self-complementary and are organized so that an energetically stable intramolecular duplex forming a T-shaped hairpin may be formed. These hairpin structures function as an origin for viral DNA replication, serving as primers for the cellular DNA polymerase complex. Following wild-type AAV infection in mammalian cells, the Rep genes (i.e., Rep78 and Rep52) are expressed from the P5 promoter and the P19 promoter, respectively, and both Rep proteins have a function in the replication of the viral genome. A splicing event in the Rep ORF results in the expression of actually four Rep proteins (i.e., Rep78, Rep68, Rep52 and Rep40). However, it has been shown that the unspliced mRNA, encoding Rep78 and Rep52 proteins, in mammalian cells are sufficient for AAV vector production. Also in insect cells the Rep78 and Rep52 proteins suffice for AAV vector production.

The AAV VP proteins are known to determine the cellular tropicity of the AAV virion. The VP protein-encoding sequences are significantly less conserved than Rep proteins and genes among different AAV serotypes. The ability of Rep and ITR sequences to cross-complement corresponding sequences of other serotypes allows for the production of pseudotyped AAV particles comprising the capsid proteins of one serotype (e.g., AAV5) and the Rep and/or ITR sequences of another AAV serotype (e.g., AAV2). Herein, a pseudotyped AAV particle may be referred to as being of the type “x/y”, where “x” indicates the source of ITRs and “y” indicates the serotype of capsid, for example a 2/5 AAV particle has ITRs from AAV2 and a capsid from AAV5.

An AAV vector can comprise one or more polynucleotide sequences of interest (one or more transgenes) that are flanked by at least one AAV ITR. Thus, in one aspect, the disclosure relates to a nucleic acid vector construct comprising one or more nucleotide sequences encoding peptide sequences of one or more wildtype or variant Aβ peptides disclosed herein, wherein the nucleic acid vector construct is a recombinant AAV vector and thus comprises at least one AAV ITR flanking the one or more nucleotide sequences encoding peptide sequences of one or more wildtype or variant Aβ peptides. In some embodiments of the nucleic acid vector construct, the one or more nucleotide sequences encoding peptide sequences of one or more wildtype or variant Aβ peptides is flanked by AAV ITRs on either side.

Any suitable serotype of AAV may be used as a vector, and the vector may comprise one or more polynucleotide sequences of interest. In some embodiments, the AAV vector comprising one or more polynucleotide sequences of interest is an AAV1, AAV2, AAV3, AAV4, AAV5, AAV6, AAV7, AAV8, AAV9, AAV10, AAV11, AAV12, AAV2.5, AAvDJ, AAVrhlO.XX, AAVrh.8, AAVrh.10, AAVrh.43, AAVpi.2, AAVhu.11, AAVhu.32, AAVhu.37, or PHP.eB AAV vector. In some embodiments, the vector is capable of crossing the blood-brain barrier. In some embodiments, the vector capable of crossing the blood-brain barrier comprises AAV9, PHP.eB, AAVrh.8, AAVrh.10, or AAVrh.43. In some embodiments, the vector capable of crossing the blood-brain barrier can be delivered, for example, intravenously, intracerebrally, and/or intraventricularly, to efficiently and widely transduce neurons in the adult CNS. In some embodiments, adenoviral vectors are modified to reduce the host response. See, e.g., Russell J. Gen. Virol. 81:2573-2604 (2000); U.S. Patent Publication No. 2008/0008690; and Zaldumbide et al., Gene Therapy 15(4):239-46(2008); all publications specifically incorporated by reference herein in their entirety.

One of skill in the art would be well equipped to construct an AAV vector comprising one or more polynucleotide sequences of interest flanked by at least one AAV ITR through standard recombinant techniques (see, for example, Maniatis et al., 1988 and Ausubel et al., 1994, both specifically incorporated by reference herein in their entirety).

Vectors can also comprise other components or functionalities that further modulate gene delivery and/or gene expression, or that otherwise provide beneficial properties to the targeted cells. Such other components include, for example, components that influence binding or targeting to cells (including components that mediate cell-type or tissue-specific binding); components that influence uptake of the vector nucleic acid by the cell; components that influence localization of the polynucleotide within the cell after uptake (such as agents mediating nuclear localization); and components that influence expression of the polynucleotide.

Such components also might include markers, such as detectable and/or selection markers that can be used to detect or select for cells that have taken up and are expressing the nucleic acid delivered by the vector. Such components can be provided as a natural feature of the vector (such as the use of certain viral vectors which have components or functionalities mediating binding and uptake), or vectors can be modified to provide such functionalities. A large variety of such vectors are known in the art and are generally available. When a vector is maintained in a host cell, the vector can either be stably replicated by the cells during mitosis as an autonomous structure, incorporated within the genome of the host cell, or maintained in the host cell's nucleus or cytoplasm.

Eukaryotic expression cassettes included in the vectors particularly contain (in a 5′-to-3′ direction) regulatory elements including a eukaryotic transcriptional promoter operably linked to a protein-coding sequence, splice signals including intervening sequences, a transcriptional termination/polyadenylation sequence, post-transcriptional regulatory elements, and origins of replication.

a. Promoter/Enhancers

A “promoter” is a control sequence that is a region of a nucleic acid sequence at which initiation and rate of transcription are controlled. It may contain genetic elements at which regulatory proteins and molecules may bind, such as RNA polymerase and other transcription factors, to initiate the specific transcription a nucleic acid sequence. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence.

A promoter generally comprises a sequence that functions to position the start site for RNA synthesis. The best known example of this is the TATA box, but in some promoters lacking a TATA box, such as, for example, the promoter for the mammalian terminal deoxynucleotidyl transferase gene and the promoter for the SV40 late genes, a discrete element overlying the start site itself helps to fix the place of initiation. Additional promoter elements regulate the frequency of transcriptional initiation. Typically, these are located in the region 30-110 bp upstream of the start site, although a number of promoters have been shown to contain functional elements downstream of the start site as well. To bring a coding sequence “under the control of” a promoter, one positions the 5′ end of the transcription initiation site of the transcriptional reading frame “downstream” of (i.e., 3′ of) the chosen promoter. The “upstream” promoter stimulates transcription of the DNA and promotes expression of the encoded RNA.

The spacing between promoter elements frequently is flexible, so that promoter function is preserved when elements are inverted or moved relative to one another. In the thymidine kinase promoter, for example, the spacing between promoter elements can be increased to 50 bp apart before activity begins to decline. Depending on the promoter, it appears that individual elements can function either cooperatively or independently to activate transcription. A promoter may or may not be used in conjunction with an “enhancer,” which refers to a cis-acting regulatory sequence involved in the transcriptional activation of a nucleic acid sequence.

A promoter may be one naturally associated with a nucleic acid sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other virus, or prokaryotic or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (see U.S. Pat. Nos. 4,683,202 and 5,928,906, each specifically incorporated by reference herein in its entirety). Furthermore, it is contemplated the control sequences that direct transcription and/or expression of sequences within non-nuclear organelles such as mitochondria and the like can be employed as well.

Naturally, it will be important to employ a promoter and/or enhancer that effectively directs the expression of the DNA segment in the organelle, cell type, tissue, organ, or organism chosen for expression. Those of skill in the art of molecular biology generally know the use of promoters, enhancers, and cell type combinations for protein expression, (see, for example Sambrook et al. 1989, specifically incorporated by reference herein in its entirety). The promoters employed may be constitutive, cell-specific, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

Additionally any promoter/enhancer combination (as per, for example, the Eukaryotic Promoter Data Base EPDB, through world wide web at epd.isb-sib.ch/) could also be used to drive expression. Non-limiting examples of other potential promoters include early or late viral promoters, such as, SV40 early or late promoters, cytomegalovirus (CMV) immediate early promoters, Rous Sarcoma Virus (RSV) early promoters; eukaryotic cell promoters, such as, e. g., beta actin promoter (Ng, 1989; Quitsche et al., 1989), GADPH promoter (Alexander et al., 1988, Ercolani et al., 1988), metallothionein promoter (Karin et al., 1989; Richards et al., 1984); and concatenated response element promoters, such as cyclic AMP response element promoters (cre), serum response element promoter (sre), phorbol ester promoter (TPA) and response element promoters (tre) near a minimal TATA box. It is also possible to use human growth hormone promoter sequences (e.g., the human growth hormone minimal promoter described at Genbank, accession no. X05244, nucleotide 283-341) or a mouse mammary tumor promoter (available from the ATCC, Cat. No. ATCC 45007). A specific example could be a phosphoglycerate kinase (PGK) promoter.

In some embodiments, expression of the polynucleotide is regulated by a constitutive promoter. In some embodiments, the constitutive promoter is CAG (also known as CAGGS or CBA), EF-1ALPHA, ubiquitin, or CMV.

In some embodiments, expression of the polynucleotide is regulated by a cell-specific promoter. In some embodiments the cell-specific promoter is a neuron-specific promoter. In some embodiments, the neuron-specific promoter comprises a human synapsin I (SYN) promoter, a mouse calcium/calmodulin-dependent protein kinase II (CaMKII) promoter, a rat tubulin alpha I (Tal), rat neuron-specific enolase (NSE) promoter, a human platelet-derived growth factor-beta chain (PDGF) promoter, or THY1 (CD90) promoter. In some embodiments, the cell-specific promoter is human synapsin I.

In some embodiments, expression of the polynucleotide is regulated by a tissue-specific promoter. In some embodiments the tissue-specific promoter is a choroid plexus-specific promoter. In some embodiments, the choroid plexus-specific promoter comprises a Prlr promoter, a Spint2 promoter, or a F5 promoter. In some embodiments, the tissue-specific promoter is a liver-specific promoter. Liver-specific promoters have been described, for example, in L. M. Kattenhorn et al., Hum. Gene Ther. 27(12):947-961 (2016), specifically incorporated by reference herein in its entirety.

b. Protease Cleavage Sites/Self-Cleaving Peptides and Internal Ribosome Binding Sites

Suitable protease cleavages sites and self-cleaving peptides are known to the skilled person (see, e.g., in Ryan et al., 1997; Scymczak et al., 2004). Examples of protease cleavage sites are the cleavage sites of furin proteases, potyvirus NIa proteases (e.g., tobacco etch virus protease), potyvirus HC proteases, potyvirus P1 (P35) proteases, byovirus Nla proteases, byovirus RNA-2-encoded proteases, aphthovirus L proteases, enterovirus 2A proteases, rhinovirus 2A proteases, picorna 3C proteases, comovirus 24K proteases, nepovirus 24K proteases, RTSV (rice tungro spherical virus) 3C-like protease, PY\IF (parsnip yellow fleck virus) 3C-like protease, thrombin, factor Xa and enterokinase. Due to its high cleavage stringency, TEV (tobacco etch virus) protease cleavage sites may be used. In some embodiments, the protease cleavage sites are the cleavage sites of furin proteases.

Exemplary self-cleaving peptides (also called “cis-acting hydrolytic elements”, CHYSEL; see deFelipe (2002) are derived from potyvirus and cardiovirus 2A peptides. Particular self-cleaving peptides may be selected from 2A peptides derived from FMDV (foot-and-mouth disease virus), equine rhinitis A virus, Thosea asigna virus, and porcine teschovirus.

A specific initiation signal also may be used for efficient translation of coding sequences in a polycistronic message. These signals include the ATG initiation codon or adjacent sequences. For example, an initiation signal may comprise a Kozak consensus sequence having an amino acid sequence comprising GCCACCAUGGG (SEQ ID NO:34). See Kozak, 1987; Harte et al., 2012. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

In certain embodiments, the use of internal ribosome entry sites (IRES) elements are used to create multigene, or polycistronic, messages. IRES elements are able to bypass the ribosome scanning model of 5′ methylated Cap dependent translation and begin translation at internal sites (Pelletier and Sonenberg, 1988). IRES elements from two members of the picornavirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message (see U.S. Pat. Nos. 5,925,565 and 5,935,819, each herein incorporated by reference).

c. Multiple Cloning Sites

Vectors can include a multiple cloning site (MCS), which is a nucleic acid region that contains multiple restriction enzyme sites, any of which can be used in conjunction with standard recombinant technology to digest the vector (see, for example, Carbonelli et al., 1999, Levenson et al., 1998, and Cocea, 1997, specifically incorporated by reference herein in their entirety). “Restriction enzyme digestion” refers to catalytic cleavage of a nucleic acid molecule with an enzyme that functions only at specific locations in a nucleic acid molecule. Many of these restriction enzymes are commercially available. Use of such enzymes is widely understood by those of skill in the art. Frequently, a vector is linearized or fragmented using a restriction enzyme that cuts within the MCS to enable exogenous sequences to be ligated to the vector. “Ligation” refers to the process of forming phosphodiester bonds between two nucleic acid fragments, which may or may not be contiguous with each other. Techniques involving restriction enzymes and ligation reactions are well known to those of skill in the art of recombinant technology.

d. Splicing Sites

Most transcribed eukaryotic RNA molecules will undergo RNA splicing to remove introns from the primary transcripts. Vectors containing genomic eukaryotic sequences may require donor and/or acceptor splicing sites to ensure proper processing of the transcript for protein expression (see, for example, Chandler et al., 1997, herein incorporated by reference.)

e. Termination Signals

The vectors or constructs may comprise at least one termination signal. A “termination signal” or “terminator” is comprised of the DNA sequences involved in specific termination of an RNA transcript by an RNA polymerase. Thus, in certain embodiments a termination signal that ends the production of an RNA transcript is contemplated. A terminator may be necessary in vivo to achieve desirable message levels.

In eukaryotic systems, the terminator region may also comprise specific DNA sequences that permit site-specific cleavage of the new transcript so as to expose a polyadenylation site. This signals a specialized endogenous polymerase to add a stretch of about 200 A residues (polyA) to the 3′ end of the transcript. RNA molecules modified with this polyA tail appear to more stable and are translated more efficiently. Thus, in other embodiments involving eukaryotes, the terminator comprises a signal for the cleavage of the RNA, and the terminator signal promotes polyadenylation of the message. The terminator and/or polyadenylation site elements can serve to enhance message levels and to minimize read through from the cassette into other sequences.

Terminators contemplated include any known terminator of transcription described herein or known to one of ordinary skill in the art, including but not limited to, for example, the termination sequences of genes, such as for example the bovine growth hormone terminator or viral termination sequences, such as for example the SV40 terminator. In certain embodiments, the termination signal may be a lack of transcribable or translatable sequence, such as due to a sequence truncation.

f. Polyadenylation Signals

In expression, particularly eukaryotic expression, one will typically include a polyadenylation signal to effect proper polyadenylation of the transcript. The nature of the polyadenylation signal is not believed to be crucial to the successful practice, and any such sequence may be employed. Exemplary embodiments include the SV40 polyadenylation signal or the bovine growth hormone polyadenylation signal, convenient and known to function well in various target cells. Polyadenylation may increase the stability of the transcript or may facilitate cytoplasmic transport.

g. Post-Transcriptional Regulatory Elements

A vector for use in the disclosure can also comprise one or more post-transcriptional regulatory elements (PREs). Examples of PREs include the woodchuck hepatitis virus PRE (WPRE), hepatitis B virus PRE, and Intron A of human cytomegalovirus immediate early gene. See Sun et al. 2009 and Mariati et al. 2010 for further examples and details. In a particular embodiment, the PRE is a WPRE. WPRE is a DNA sequence that, when transcribed, creates a tertiary structure to enhance expression of genes delivered by viral vectors.

h. Origins of Replication

In order to propagate a vector in a host cell, the vector may contain one or more origins of replication sites (often termed “ori”), for example, a nucleic acid sequence corresponding to oriP of EBV as described above or a genetically engineered oriP with a similar or elevated function in differentiation programming, which is a specific nucleic acid sequence at which replication is initiated. Alternatively a replication origin of other extra-chromosomally replicating virus as described above or an autonomously replicating sequence (ARS) can be employed.

3. Vector Delivery

Genetic modification or introduction of exogenous nucleic acids into cells may use any suitable methods for nucleic acid delivery for transformation of a cell, as described herein or as would be known to one of ordinary skill in the art. Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Such methods include, but are not limited to, direct delivery of DNA such as by ex vivo transfection (Wilson et al., 1989, Nabel et al, 1989); transduction; viral transduction; injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harland and Weintraub, 1985; U.S. Pat. No. 5,789,215, incorporated herein by reference); by electroporation (U.S. Pat. No. 5,384,253, incorporated herein by reference; Tur-Kaspa et al., 1986; Potter et al., 1984); by calcium phosphate precipitation (Graham and Van Der Eb, 1973; Chen and Okayama, 1987; Rippe et al., 1990); by using DEAE-dextran followed by polyethylene glycol (Gopal, 1985); by direct sonic loading (Fechheimer et al., 1987); by nucleofection; by lipofection or liposome mediated transfection (Nicolau and Sene, 1982; Fraley et al., 1979; Nicolau et al., 1987; Wong et al., 1980; Kaneda et al., 1989; Kato et al., 1991) and receptor-mediated transfection (Wu and Wu, 1987; Wu and Wu, 1988); by microprojectile or nanoparticle bombardment (PCT Application Nos. WO 94/09699 and 95/06128; U.S. Pat. Nos. 5,610,042; 5,322,783 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); by Agrobacterium-mediated transformation (U.S. Pat. Nos. 5,591,616 and 5,563,055, each incorporated herein by reference); by PEG-mediated transformation of protoplasts (Omirulleh et al., 1993; U.S. Pat. Nos. 4,684,611 and 4,952,500, each incorporated herein by reference); by desiccation/inhibition-mediated DNA uptake (Potrykus et al., 1985); thermal shock (Froger and Hall, 2007); and any combination of such methods. Through the application of techniques such as these, organelle(s), cell(s), tissue(s) or organism(s) may be stably or transiently transformed.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art (see, e.g., Sambrook et al. (2001) Molecular Cloning: A Laboratory Manual, Cold Spring Harbor Laboratory, New York).

Biological methods for introducing a polynucleotide of interest into a host cell can include the use of DNA and RNA vectors into which the polynucleotide of interest, or transgene, can be inserted. Viral vectors have become the most widely used method for inserting genes into mammalian, e.g., human cells. Other viral vectors can be derived from lentivirus, poxviruses, herpes simplex virus I, adenoviruses and adeno-associated viruses, and the like (see, e.g., U.S. Pat. Nos. 5,350,674 and 5,585,362, and the like).

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. Also contemplated are nanoparticles. An illustrative colloidal system for use as a delivery vehicle in vitro and in vivo is a liposome (e.g., an artificial membrane vesicle).

Gene therapy methods and methods of delivering genes to subjects, for example using adeno-associated viruses, are described in U.S. Pat. No. 6,967,018, WO2014/093622, US2008/0175845, US 2014/0100265, EP2432490, EP2352823, EP2384200, WO2014/127198, WO2005/122723, WO2008/137490, WO2013/1421 14, WO2006/128190, WO2009/134681, EP2341068, WO2008/027084, WO2009/054994, WO2014059031, U.S. Pat. No. 7,977,049 and WO 2014/059029, each of which are specifically incorporated herein by reference in their entirety.

a. Liposome-Mediated Transfection

One illustrative delivery vehicle is a lipid and/or a liposome. The use of lipid formulations is contemplated for the introduction of the nucleic acids into a host cell (in vitro, ex vivo, or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

In a certain embodiment, a nucleic acid may be entrapped in a lipid complex such as, for example, a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). The amount of liposomes used may vary upon the nature of the liposome as well as the cell used, for example, about 5 to about 20 μg vector DNA per 1 to 10 million of cells may be contemplated.

Liposome-mediated nucleic acid delivery and expression of foreign DNA in vitro has been very successful (Nicolau and Sene, 1982; Fraley et al., 1979; Nicolau et al., 1987). The feasibility of liposome-mediated delivery and expression of foreign DNA in cultured chick embryo, HeLa and hepatoma cells has also been demonstrated (Wong et al., 1980).

In certain embodiments, a liposome may be complexed with a hemagglutinating virus (HVJ). This has been shown to facilitate fusion with the cell membrane and promote cell entry of liposome-encapsulated DNA (Kaneda et al., 1989). In other embodiments, a liposome may be complexed or employed in conjunction with nuclear non-histone chromosomal proteins (HMG-1) (Kato et al., 1991). In yet further embodiments, a liposome may be complexed or employed in conjunction with both HVJ and HMG-1. In other embodiments, a delivery vehicle may comprise a ligand and a liposome.

In various embodiments lipids suitable for use can be obtained from commercial sources. For example, lipofectamine can be obtained from Thermo Fisher Scientific, Waltham, Mass.; dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids may be obtained from Avanti Polar Lipids, Inc. (Birmingham, Ala.). Stock solutions of lipids in chloroform or chloroform/methanol can be stored at about −20° C. Chloroform can be used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al. (1991) Glycobiology 5: 505-510). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

b. Electroporation

In certain embodiments, a nucleic acid is introduced into a cell via electroporation. Electroporation involves the exposure of a suspension of cells and DNA to a high-voltage electric discharge. Recipient cells can be made more susceptible to transformation by mechanical wounding. Also the amount of vectors used may vary upon the nature of the cells used, for example, about 5 to about 20 μg vector DNA per 1 to 10 million of cells may be contemplated.

Transfection of eukaryotic cells using electroporation has been quite successful. Mouse pre-B lymphocytes have been transfected with human kappa-immunoglobulin genes (Potter et al., 1984), and rat hepatocytes have been transfected with the chloramphenicol acetyltransferase gene (Tur-Kaspa et al., 1986) in this manner.

c. Calcium Phosphate

In other embodiments, a nucleic acid is introduced to the cells using calcium phosphate precipitation. Human KB cells have been transfected with adenovirus 5 DNA (Graham and Van Der Eb, 1973) using this technique. Also in this manner, mouse L(A9), mouse C127, CHO, CV-1, BHK, NIH3T3 and HeLa cells were transfected with a neomycin marker gene (Chen and Okayama, 1987), and rat hepatocytes were transfected with a variety of marker genes (Rippe et al., 1990).

d. DEAE-Dextran

In another embodiment, a nucleic acid is delivered into a cell using DEAE-dextran followed by polyethylene glycol. In this manner, reporter plasmids were introduced into mouse myeloma and erythroleukemia cells (Gopal, 1985).

4. Selectable or Screenable Markers

Regardless of the method used to introduce exogenous nucleic acids into a host cell or otherwise expose a cell to the inhibitor of the present disclosure, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the disclosure.

In certain embodiments, cells containing an exogenous nucleic acid may be identified in vitro or in vivo by including a marker in the expression vector or the exogenous nucleic acid. Such markers would confer an identifiable change to the cell permitting easy identification of cells containing the expression vector. Generally, a selection marker may be one that confers a property that allows for selection. A positive selection marker may be one in which the presence of the marker allows for its selection, while a negative selection marker is one in which its presence prevents its selection. An example of a positive selection marker is a drug resistance marker.

In addition to markers conferring a phenotype that allows for the discrimination of transformants based on the implementation of conditions, other types of markers including screenable markers such as GFP, whose basis is colorimetric analysis, are also contemplated. Alternatively, screenable enzymes as negative selection markers such as herpes simplex virus thymidine kinase (tk) or chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selection and screenable markers are well known to one of skill in the art.

Selectable markers may include a type of reporter gene used in laboratory microbiology, molecular biology, and genetic engineering to indicate the success of a transfection or other procedure meant to introduce foreign DNA into a cell. Selectable markers are often antibiotic resistance genes; cells that have been subjected to a procedure to introduce foreign DNA are grown on a medium containing an antibiotic, and those cells that can grow have successfully taken up and expressed the introduced genetic material. Examples of selectable markers include: the Abicr gene or Neo gene from Tn5, which confers antibiotic resistance to geneticin.

A screenable marker may comprise a reporter gene, which allows the researcher to distinguish between wanted and unwanted cells. Certain embodiments of the present invention utilize reporter genes to indicate specific cell lineages. For example, the reporter gene can be located within expression elements and under the control of the ventricular- or atrial-selective regulatory elements normally associated with the coding region of a ventricular- or atrial-selective gene for simultaneous expression. A reporter allows the cells of a specific lineage to be isolated without placing them under drug or other selective pressures or otherwise risking cell viability.

Examples of such reporters include genes encoding cell surface proteins (e.g., CD4, HA epitope), fluorescent proteins, antigenic determinants and enzymes (e.g., β-galactosidase). The vector containing cells may be isolated, e.g., by FACS using fluorescently-tagged antibodies to the cell surface protein or substrates that can be converted to fluorescent products by a vector encoded enzyme.

In specific embodiments, the reporter gene is a fluorescent protein. A broad range of fluorescent protein genetic variants have been developed that feature fluorescence emission spectral profiles spanning almost the entire visible light spectrum (see Table 1 for non-limiting examples). Mutagenesis efforts in the original Aequorea victoria jellyfish green fluorescent protein have resulted in new fluorescent probes that range in color from blue to yellow, and are some of the most widely used in vivo reporter molecules in biological research. Longer wavelength fluorescent proteins, emitting in the orange and red spectral regions, have been developed from the marine anemone, Discosoma striata, and reef corals belonging to the class Anthozoa. Still other species have been mined to produce similar proteins having cyan, green, yellow, orange, and deep red fluorescence emission. Developmental research efforts are ongoing to improve the brightness and stability of fluorescent proteins, thus improving their overall usefulness.

TABLE
Fluorescent Protein Properties

| | | | | | | Relative |
|---|---|---|---|---|---|---|
| | Excitation | Emission | Molar | | | Brightness |
| Protein | Maximum | Maximum | Extinction | Quantum | in vivo | (% of |
| (Acronym) | (nm) | (nm) | Coefficient | Yield | Structure | EGFP) |
| GFP (wt) | 395/475 | 509 | 21,000 | 0.77 | Monomer* | 48 |

Green Fluorescent Proteins

| | | | | | | |
|---|---|---|---|---|---|---|
| EGFP | 484 | 507 | 56,000 | 0.60 | Monomer* | 100 |
| AcGFP | 480 | 505 | 50,000 | 0.55 | Monomer* | 82 |
| TurboGFP | 482 | 502 | 70,000 | 0.53 | Monomer* | 110 |
| Emerald | 487 | 509 | 57,500 | 0.68 | Monomer* | 116 |
| Azami | 492 | 505 | 55,000 | 0.74 | Monomer | 121 |
| Green | | | | | | |
| ZsGreen | 493 | 505 | 43,000 | 0.91 | Tetramer | 117 |

Blue Fluorescent Proteins

| | | | | | | |
|---|---|---|---|---|---|---|
| EBFP | 383 | 445 | 29,000 | 0.31 | Monomer* | 27 |
| Sapphire | 399 | 511 | 29,000 | 0.64 | Monomer* | 55 |
| T-Sapphire | 399 | 511 | 44,000 | 0.60 | Monomer* | 79 |

Cyan Fluorescent Proteins

| | | | | | | |
|---|---|---|---|---|---|---|
| ECFP | 439 | 476 | 32,500 | 0.40 | Monomer* | 39 |
| mCFP | 433 | 475 | 32,500 | 0.40 | Monomer | 39 |
| Cerulean | 433 | 475 | 43,000 | 0.62 | Monomer* | 79 |
| CyPet | 435 | 477 | 35,000 | 0.51 | Monomer* | 53 |
| AmCyan1 | 458 | 489 | 44,000 | 0.24 | Tetramer | 31 |
| Midori-Ishi | 472 | 495 | 27,300 | 0.90 | Dimer | 73 |
| Cyan | | | | | | |
| mTFP1 | 462 | 492 | 64,000 | 0.85 | Monomer | 162 |
| (Teal) | | | | | | |

Yellow Fluorescent Proteins

| | | | | | | |
|---|---|---|---|---|---|---|
| EYFP | 514 | 527 | 83,400 | 0.61 | Monomer* | 151 |
| Topaz | 514 | 527 | 94,500 | 0.60 | Monomer* | 169 |
| Venus | 515 | 528 | 92,200 | 0.57 | Monomer* | 156 |
| mCitrine | 516 | 529 | 77,000 | 0.76 | Monomer | 174 |
| YPet | 517 | 530 | 104,000 | 0.77 | Monomer* | 238 |
| PhiYFP | 525 | 537 | 124,000 | 0.39 | Monomer* | 144 |
| ZsYellow1 | 529 | 539 | 20,200 | 0.42 | Tetramer | 25 |
| mBanana | 540 | 553 | 6,000 | 0.7 | Monomer | 13 |

Orange and Red Fluorescent Proteins

| | | | | | | |
|---|---|---|---|---|---|---|
| Kusabira | 548 | 559 | 51,600 | 0.60 | Monomer | 92 |
| Orange | | | | | | |
| mOrange | 548 | 562 | 71,000 | 0.69 | Monomer | 146 |
| dTomato | 554 | 581 | 69,000 | 0.69 | Dimer | 142 |
| dTomato- | 554 | 581 | 138,000 | 0.69 | Monomer | 283 |
| Tandem | | | | | | |
| DsRed | 558 | 583 | 75,000 | 0.79 | Tetramer | 176 |
| DsRed2 | 563 | 582 | 43,800 | 0.55 | Tetramer | 72 |
| DsRed- | 555 | 584 | 38,000 | 0.51 | Tetramer | 58 |
| Express (T1) | | | | | | 10 |
| DsRed- | 556 | 586 | 35,000 | 0.10 | Monomer | |
| Monomer | | | | | | |
| mTangerine | 568 | 585 | 38,000 | 0.30 | Monomer | 34 |
| mStrawberry | 574 | 596 | 90,000 | 0.29 | Monomer | 78 |
| AsRed2 | 576 | 592 | 56,200 | 0.05 | Tetramer | 8 |
| mRFP1 | 584 | 607 | 50,000 | 0.25 | Monomer | 37 |
| JRed | 584 | 610 | 44,000 | 0.20 | Dimer | 26 |
| mCherry | 587 | 610 | 72,000 | 0.22 | Monomer | 47 |
| HcRed1 | 588 | 618 | 20,000 | 0.015 | Dimer | 1 |
| mRaspberry | 598 | 625 | 86,000 | 0.15 | Monomer | 38 |
| HcRed- | 590 | 637 | 160,000 | 0.04 | Monomer | 19 |
| Tandem | | | | | | 12 |
| mPlum | 590 | 649 | 41,000 | 0.10 | Monomer | |
| AQ143 | 595 | 655 | 90,000 | 0.04 | Tetramer | 11 |

*Weak Dimer

### III. Neurodegenerative Disease Treatment

Aspects of the present disclosure are directed to compositions and methods of using such compositions to treat or prevent a subject suffering from a neurodegenerative disease, disorder, or condition. In some embodiments, the neurodegenerative disease is Alzheimer's disease, Parkinson's disease, Parkinson's disease dementia, vascular dementia, cerebral amyloid angiopathy, dementia with Lewy bodies, chronic traumatic encephalopathy, Down syndrome, and/or pathological aging. In some embodiments, the neurodegenerative disease is Alzheimer's disease.

In certain embodiments, the disclosed methods further comprise treating a subject who has been diagnosed with a neurodegenerative disease, disorder, or condition. In certain embodiments, the disclosed methods further comprise treating a subject who has been diagnosed as having symptoms of a neurodegenerative disease, disorder, or condition. In certain embodiments, the disclosed methods further comprise treating a subject who has been identified as being at risk of having a neurodegenerative disease, disorder, or condition. A subject may be diagnosed with or as having symptoms of or may be identified as being at risk of having a neurodegenerative disease, disorder, or condition using tests and diagnostic methods known in the art and described herein.

In some embodiments, the methods further comprise determining a subject is in need of treatment comprising a therapeutically effective amount of a composition comprising a vector encoding a wild-type Aβ peptide or an Aβ peptide variant or a fragment or functional derivative thereof. The vector encoding an Aβ peptide may encode a minigene that enables protein expression of the Aβ peptide, and in some embodiments, the minigene may comprise a nucleotide sequence corresponding to an amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, or any value derivable therein, with any of SEQ ID NO:8, SEQ ID NOs:13-17, SEQ ID NOs:35-38, or SEQ ID NOs:66-85. In some embodiments, the methods further comprise providing to a subject a treatment comprising a therapeutically effective amount of a composition comprising a vector encoding a wild-type Aβ peptide or an Aβ peptide variant or a fragment or functional derivative thereof when it is determined that the subject is in need thereof. In some embodiments, determining a subject is in need of a treatment comprising a therapeutically effective amount of a composition comprising a vector encoding a wild-type Aβ peptide or an Aβ peptide variant or a fragment or functional derivative thereof comprises diagnosing the subject with a neurodegenerative disease, disorder, or condition. In some embodiments, determining a subject is in need of a treatment comprising a therapeutically effective amount of a composition comprising a vector encoding a wild-type Aβ peptide or an Aβ peptide variant or a fragment or functional derivative thereof comprises diagnosing the subject as having symptoms of a neurodegenerative disease, disorder, or condition. In some embodiments, determining a subject is in need of a treatment comprising a therapeutically effective amount of a composition comprising a vector encoding a wild-type Aβ peptide or an Aβ peptide variant or a fragment or functional derivative thereof comprises identifying the subject as being at risk of having a neurodegenerative disease, disorder, or condition.

In some embodiments, the disclosed methods comprise administering to a subject suffering from a neurodegenerative disease, disorder, or condition a therapeutically effective amount of a composition comprising a vector encoding a wild-type Aβ peptide or an Aβ peptide variant or a fragment or functional derivative thereof. As disclosed herein, neurodegenerative diseases can be associated with aggregation or oligomerization of Aβ peptide, and administration of a vector encoding an Aβ peptide variant or a fragment or functional derivative thereof has been surprising and unexpectedly found to prevent or decease formation of endogenous Aβ peptide oligomers, protofibrils, fibrils, or plaques. Further, administering a vector encoding an Aβ peptide variant or a fragment or functional derivative thereof can surprisingly prevent or decrease cytotoxicity of endogenous Aβ peptide aggregate. Accordingly, in some embodiments, disclosed are compositions and corresponding method for treating a subject suffering from neurodegenerative disease, disorder, or condition with a therapeutically effective amount of a composition comprising a vector encoding an Aβ peptide variant or a fragment or functional derivative thereof. In further embodiments, disclosed is a method for of inhibiting aggregation of endogenous Aβ peptide in vivo, comprising contacting at least one such peptide with a therapeutically effective amount of an expressed Aβ peptide variant from a vector encoding the Aβ peptide variant, wherein the vector is optionally comprised in a composition. In further embodiments, disclosed is a method for of promoting aggregation of endogenous Aβ peptide in vivo, comprising contacting at least one such peptide with a therapeutically effective amount of an expressed wild-type Aβ peptide or an Aβ peptide variant from a vector encoding the wild-type Aβ peptide or Aβ peptide variant, wherein the vector is optionally comprised in a composition. In some embodiments, the neurodegenerative disease, disorder, or condition is one which is characterized by aberrant aggregation of endogenous Aβ peptide. In some embodiments, the neurodegenerative disease, disorder, or condition is Alzheimer's disease.

The subject may have a condition that has as a symptom and/or a mechanism an aberrant aggregation of Aβ peptide, for example. Embodiments of the disclosure include treatment or prevention of any medical condition in which modulation of Aβ peptide aggregation would be beneficial. In specific embodiments, an individual is provided a therapeutically effective amount of one or more compositions comprising a vector encoding an Aβ peptide variant or a fragment or functional derivative thereof for attenuation of Aβ peptide aggregation in an individual or a delay or reversal in Aβ peptide aggregation in an individual. In specific embodiments, an individual is provided a therapeutically effective amount of one or more compositions comprising a vector encoding a wild-type Aβ peptide or an Aβ peptide variant or a fragment or functional derivative thereof for promotion of Aβ peptide aggregation in an individual. In specific embodiments, the medical condition treated or prevented with compositions comprising a vector encoding an Aβ peptide variant or a fragment or functional derivative thereof comprises neurodegenerative diseases, disorders, or conditions which are characterized by Aβ peptide aggregation. In particular embodiments, Aβ peptide aggregation is not treated with compositions the disclosure. In some cases, the compositions comprising a vector encoding an Aβ peptide variant or a fragment or functional derivative thereof treats or prevents the medical condition in the individual by ameliorating, inhibiting, delaying, or reversing Aβ peptide aggregation, for example. In some cases, the compositions comprising a vector encoding a wild-type Aβ peptide or an Aβ peptide variant or a fragment or functional derivative thereof treats or prevents the medical condition in the individual by promoting Aβ peptide aggregation, for example. In some embodiments, ameliorating, inhibiting, delaying, or reversing Aβ peptide aggregation may dampen tau protein seeding.

The subject may have a condition that has as a symptom and/or a mechanism aberrant amyloid plaque formation, protein misfolding, increases in tau protein levels and/or levels of phosphorylated tau protein, increases in seeding of tau protein and/or Aβ peptide seeding, neuroinflammation, cognitive decline, neurodegeneration, neuronal loss, and/or synaptic loss, for example. Embodiments of the disclosure include treatment or prevention of any medical condition in which modulation of amyloid plaque formation, protein misfolding, tau protein levels, tau protein phosphorylation, rates of seeding of tau protein and/or Aβ peptide seeding, neuroinflammation, cognitive decline, neurodegeneration, neuronal loss, and/or synaptic loss would be beneficial. In specific embodiments, an individual is provided a therapeutically effective amount of one or more compositions comprising a vector encoding an Aβ peptide variant or a fragment or functional derivative thereof for attenuation of amyloid plaque formation, protein misfolding, tau protein levels, tau protein phosphorylation, rates of seeding of tau protein and/or Aβ peptide seeding, neuroinflammation, cognitive decline, neurodegeneration, neuronal loss, and/or synaptic loss in an individual. In specific embodiments, the medical condition treated or prevented with compositions comprising a vector encoding a wild-type Aβ peptide or an Aβ peptide variant or a fragment or functional derivative thereof comprises neurodegenerative diseases, disorders, or conditions which can lead to protein misfolding, endogenous Aβ peptide aggregation, amyloid plaque formation, neuroinflammation, neurodegeneration, neuronal loss, or synaptic loss; aberrant tau levels, phosphorylation of tau, or phosphorylated tau levels; seeding of tau or seeding of endogenous Aβ peptide; and/or cognitive decline. In particular embodiments, protein misfolding, endogenous Aβ peptide aggregation, amyloid plaque formation, neuroinflammation, neurodegeneration, neuronal loss, or synaptic loss; aberrant tau levels, phosphorylation of tau, or phosphorylated tau levels; seeding of tau or seeding of endogenous Aβ peptide; and/or cognitive decline is not treated with compositions of the disclosure. In some cases, the compositions comprising a vector encoding an Aβ peptide variant or a fragment or functional derivative thereof treats or prevents the medical condition in the individual by ameliorating or inhibiting protein misfolding, endogenous Aβ peptide aggregation, amyloid plaque formation, neuroinflammation, neurodegeneration, neuronal loss, or synaptic loss; aberrant tau levels, phosphorylation of tau, or phosphorylated tau levels; seeding of tau or seeding of endogenous Aβ peptide; and/or cognitive decline, for example.

Embodiments of the disclosure include compositions and methods that treat or prevent protein misfolding, endogenous Aβ peptide aggregation, amyloid plaque formation, neuroinflammation, neurodegeneration, neuronal loss, or synaptic loss; aberrant tau levels, phosphorylation of tau, or phosphorylated tau levels; seeding of tau or seeding of endogenous Aβ peptide; and/or cognitive decline as a result of neurodegenerative disorders. In specific cases, delivery of compositions comprising a vector encoding an Aβ peptide variant or a fragment or functional derivative thereof to an individual prevents or decreases formation of endogenous Aβ peptide oligomers, protofibrils, fibrils, or plaques or cytotoxicity of endogenous Aβ peptide aggregate. In specific cases, delivery of compositions comprising a vector encoding a wild-type Aβ peptide or an Aβ peptide variant or a fragment or functional derivative thereof to an individual promotes formation of endogenous Aβ peptide oligomers, protofibrils, fibrils, or plaques of endogenous Aβ peptide aggregate.

In some embodiments, a vector encoding wild-type Aβ peptide comprises an amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, or any value derivable therein, with DAEFRHDSGYEVHHQKLVFFAEDVGSNKGAIIGLMVGGVVIA (SEQ ID NO:2), or a fragment or functional derivative thereof. In some embodiments, a wild-type Aβ peptide of the disclosure comprises, consists of, or consists essentially of SEQ ID NO:2, or a fragment or functional derivative thereof. In some embodiments, the fragment or functional derivative of SEQ ID NO:2 comprises an amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, or any value derivable therein, with KLVFFAE (SEQ ID NO:39).

In some embodiments, a vector encoding an Aβ peptide variant or a fragment or functional derivative thereof comprises variants of SEQ ID NO:2, corresponding to the wild-type Aβ peptide amino acid sequence.

In some embodiments, an Aβ peptide variant comprises an amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, or any value derivable therein, with DAEFRHDSGYEVHHQKLVDFAEDVGSNKGAIIGPMVGGVVIA (SEQ ID NO:3) or a fragment or functional derivative thereof. In some embodiments, an Aβ peptide variant of the disclosure comprises, consists of, or consists essentially of SEQ ID NO:3 or a fragment or functional derivative thereof. In some embodiments, the fragment or functional derivative of SEQ ID NO:3 comprises an amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, or any value derivable therein, with KLVDFAE (SEQ ID NO:10).

In some embodiments, an Aβ peptide variant comprises an amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, or any value derivable therein, with DAEFRHDSGYEVHHQKLVFPAEDVGSNKGAIIGLMVGGVVIA (SEQ ID NO:4) or a fragment or functional derivative thereof. In some embodiments, an Aβ peptide variant of the disclosure comprises, consists of, or consists essentially of SEQ ID NO:4 or a fragment or functional derivative thereof. In some embodiments, the fragment or functional derivative of SEQ ID NO:4 comprises an amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, or any value derivable therein, with KLVFPAE (SEQ ID NO:11).

In some embodiments, an Aβ peptide variant comprises an amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, or any value derivable therein, with DAEFRHDSGYEVHHQKLVDFAEDVGSNKGAIIGLMVGGVVIA (SEQ ID NO:5) or a fragment or functional derivative thereof. In some embodiments, an Aβ peptide variant of the disclosure comprises, consists of, or consists essentially of SEQ ID NO:5 or a fragment or functional derivative thereof. In some embodiments, the fragment or functional derivative of SEQ ID NO:5 comprises an amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, or any value derivable therein, with KLVDFAE (SEQ ID NO:10).

In some embodiments, an Aβ peptide variant comprises an amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, or any value derivable therein, with DAEFRHDSGYEVHHQKLVPFAEDVGSNKGAIIGLMVGGVVIA (SEQ ID NO:6) or a fragment or functional derivative thereof. In some embodiments, an Aβ peptide variant of the disclosure comprises, consists of, or consists essentially of SEQ ID NO:6 or a fragment or functional derivative thereof. In some embodiments, the fragment or functional derivative of SEQ ID NO:6 comprises an amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, or any value derivable therein, with KLVPFAE (SEQ ID NO:12).

In some embodiments, an Aβ peptide variant comprises an amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, or any value derivable therein, with DAEFRHDSGYEVHHQKLPFFAEDVGSNKGAIIGLMVGGVVIA (SEQ ID NO:7), or a fragment or functional derivative thereof. In some embodiments, an Aβ peptide variant of the disclosure comprises, consists of, or consists essentially of SEQ ID NO:7, or a fragment or functional derivative thereof. In some embodiments, the fragment or functional derivative of SEQ ID NO:7 comprises an amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, or any value derivable therein, with KLPFFAE (SEQ ID NO:65). In some embodiments, the Aβ peptide variant does not comprise, consist of, or consist essentially of SEQ ID NO:7.

In some embodiments, a wildtype Aβ peptide or an Aβ peptide variant comprising, consisting of, or consisting essentially of SEQ ID NO:2, SEQ ID NO:3, SEQ ID NO:4, SEQ ID NO:5, SEQ ID NO:6, or SEQ ID NO:7 comprises a N-terminal truncation. In some embodiments, the N-terminal truncation comprises a 1 to 22 amino acid truncation. In some embodiments, the N-terminal truncation comprises a 1, 2, 3, 4, 5, 6, 7, 8, 9, 10, 11, 12, 13, 14, 15, 16, 17, 18, 19, 20, 21, or 22 amino acid truncation.

In some embodiments, a wildtype Aβ peptide or an Aβ peptide variant comprising, consisting of, or consisting essentially of SEQ ID NO:2, SEQ ID NO:3, SEQ ID NO:4, SEQ ID NO:5, SEQ ID NO:6, or SEQ ID NO:7 comprises a C-terminal truncation. In some embodiments, the C-terminal truncation comprises a 1 to 27 amino acid truncation. In some embodiments, the C-terminal truncation comprises a 1, 2, 3, 4, 5, 6, 7, 8, 9, 10, 11, 12, 13, 14, 15, 16, 17, 18, 19, 20, 21, 22, 23, 24, 25, 26, 27, or 28 amino acid truncation.

In some embodiments, a wildtype Aβ peptide or an Aβ peptide variant comprising, consisting of, or consisting essentially of SEQ ID NO:2, SEQ ID NO:3, SEQ ID NO:4, SEQ ID NO:5, SEQ ID NO:6, or SEQ ID NO:7 comprises a N-terminal truncation and a C-terminal truncation. In some embodiments, the N-terminal truncation comprises a 1 to 22 amino acid truncation and the C-terminal truncation comprises a 1 to 27 amino acid truncation. In some embodiments, the N-terminal truncation comprises a 1, 2, 3, 4, 5, 6, 7, 8, 9, 10, 11, 12, 13, 14, 15, 16, 17, 18, 19, 20, 21, or 22 amino acid truncation, and the C-terminal truncation comprises a 1, 2, 3, 4, 5, 6, 7, 8, 9, 10, 11, 12, 13, 14, 15, 16, 17, 18, 19, 20, 21, 22, 23, 24, 25, 26, 27, or 28 amino acid truncation.

The wildtype Aβ peptide or Aβ peptide variant may be encoded by a vector encoding a minigene that enables protein expression of Aβ peptide. In some embodiments, the minigene may comprise, for example, a nucleotide sequence corresponding to an amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, or any value derivable therein, with any of SEQ ID NO:8, SEQ ID NOs:13-17, SEQ ID NOs:35-38, or SEQ ID NOs:66-85.

Also disclosed in some embodiments are pharmaceutical compositions comprising a vector encoding a wild-type Aβ peptide or an Aβ peptide variant or a fragment or functional derivative thereof. Although in some cases the compositions comprising a vector encoding a wild-type Aβ peptide or an Aβ peptide variant or a fragment or functional derivative thereof are provided as a sole therapy for the individual, in other cases the individual is provided one or more additional therapies for treating or preventing protein misfolding, endogenous Aβ peptide aggregation, amyloid plaque formation, neuroinflammation, neurodegeneration, neuronal loss, or synaptic loss; aberrant tau levels, phosphorylation of tau, or phosphorylated tau levels; seeding of tau or seeding of endogenous Aβ peptide; and/or cognitive decline, or in other cases the individual is provided one or more additional therapies for promoting Aβ peptide aggregation, amyloid plaque formation, and/or fibril formation.

The one or more additional therapies may be of any kind, but in specific cases the one or more additional therapies are one or more additional neurodegenerative disease therapeutics, for example, Alzheimer's disease medications. In some embodiments, the Alzheimer's disease medications comprise aducanumab, donepezil, rivastigmine, galantamine, memantine, or tacrine. In some embodiments, Alzheimer's disease medications may comprise additional and or alternative drugs and biologics to: reduce Aβ levels or prevent aggregation (i.e., secretase inhibitors/modulators, anti-Aβ passive immunization, anti-Aβ active immunization, or metal chelators); lower tau levels or prevent aggregation of tau or diminish pathologic phosphorylation of tau (i.e., kinase inhibitors, anti-tau passive immunization, anti-tau active immunization, or antisense oligonucleotides); stabilize microtubules; diminish neurodegeneration; block or modify inflammatory responses; diminish neuropsychiatric symptoms; enhance cognition (i.e. neurotransmitter inhibitors, modulators, activators); preserve or improve vascular function; or alter cellular metabolism. See, e.g., K. G. Yiannopoulou & S. G. Papageorgiou, J. Cent. Nerv. Sys. Dis. 12:1179573520907397 (2020), and J. Cummings et al., Alzheimer's Dement. (N.Y.) 5:272-293 (2019).

In some embodiments, the one or more additional therapies comprise one or more therapies to treat disorders or co-morbidities of aging, for example, cardiovascular diseases, diabetes, atherosclerosis, obesity, cancer, infection, and neurological disorders. Any well-established indicators of aging progression can be used. In some embodiments, the one or more therapies to treat disorders or co-morbidities of aging have the effect of: reducing the incidence of cancer, delaying or ameliorating cardiovascular disease, such as atherosclerosis; delaying and/or ameliorating osteoporosis; improving glucose tolerance or reducing incidence of related diseases, such as diabetes and obesity; improving or reducing the decline in memory function and other cognitive functions; improving or reducing the decline neuromuscular coordination; and improving or reducing the decline in immune function. The amelioration of age-related disorders can be as a result of reduction of symptoms in an affected subject or a reduction of incidence of the disease or disorder in a population as compared to an untreated population. The one or more therapies have the effect of treating and/or preventing various age-related conditions and diseases, as assessed by particular markers and disorders of aging. In a further aspect, therefore, the invention refers to the treatment or prevention in a subject of at least a disorder or marker of aging that is selected from the group of reduced cardiovascular function, osteoporosis, arthrosis, glucose intolerance, insulin resistance, loss of memory, loss of neuromuscular coordination, increase in cardiovascular disease, decrease in heart, circulatory, or lung function and decrease in longevity, or combinations thereof.

### IV. Administration of Therapeutic Compositions

In particular, embodiments, the compositions comprising a vector encoding a wild-type Aβ peptide or an Aβ peptide variant or a fragment or functional derivative thereof are formulated as a pharmaceutical composition for therapeutic administration. In some embodiments, the disclosed methods comprise administering a neurodegenerative disease therapy to a subject or patient. The compositions of the disclosure may be used for in vivo, in vitro, or ex vivo administration.

In some embodiments, the neurodegenerative disease therapy comprises a protein-based therapy, which may be a wild-type Aβ peptide or an Aβ peptide variant therapy. In some embodiments, the neurodegenerative disease therapy comprises a polynucleotide-based therapy, which may be a therapy including a vector encoding a wild-type Aβ peptide or an Aβ peptide variant or a fragment or functional derivative thereof. In some embodiments, the neurodegenerative disease therapy comprises one or more neurodegenerative disease medications. Any of these neurodegenerative disease therapies may be excluded. Combinations of these therapies may also be administered.

The therapy provided herein may comprise administration of a combination of therapeutic compositions, such as a first neurodegenerative disease therapy (e.g., a wild-type Aβ peptide or an Aβ peptide variant or a polynucleotide encoding a wild-type Aβ peptide or an Aβ peptide variant) and one or more additional neurodegenerative disease therapies (e.g., neurodegenerative disease medications). The therapies may be administered in any suitable manner known in the art. For example, the first and one or more additional neurodegenerative disease therapies may be administered sequentially (at different times) or concurrently (at the same time or approximately the same time; also “simultaneously” or “substantially simultaneously”). In some embodiments, the first and one or more additional neurodegenerative disease therapies may be administered in a separate composition. In some embodiments, the first and one or more additional neurodegenerative disease therapies may be in the same composition. The different therapies may be administered in one composition or in more than one composition, such as 2 compositions, 3 compositions, or 4 compositions. Various combinations of the agents may be employed.

In some embodiments, the composition(s) comprising a vector encoding a wild-type Aβ peptide or an Aβ peptide variant or a fragment or functional derivative thereof and the one or more additional neurodegenerative disease medications are administered substantially simultaneously. In some embodiments, the composition(s) comprising a vector encoding a wild-type Aβ peptide or an Aβ peptide variant or a fragment or functional derivative thereof and the one or more additional neurodegenerative disease medications are administered sequentially. In some embodiments, the composition(s) comprising a vector encoding a wild-type Aβ peptide or an Aβ peptide variant or a fragment or functional derivative thereof is administered before administering the one or more additional neurodegenerative disease medications. In some embodiments, the composition(s) comprising a vector encoding a wild-type Aβ peptide or an Aβ peptide variant or a fragment or functional derivative thereof is administered after administering the one or more additional neurodegenerative disease medications.

In some embodiments, the composition(s) comprising a vector encoding a wild-type Aβ peptide or an Aβ peptide variant or a fragment or functional derivative thereof is delivered to the subject a single time. In some embodiments, the composition comprising a vector encoding a wild-type Aβ peptide or an Aβ peptide variant or a fragment or functional derivative thereof is delivered to the subject multiple times, such as once a day, more than once a day, once a week, more than once a week, once a month, more than once a month, once a year, or more than once a year. In some embodiments, a wild-type Aβ peptide or an Aβ peptide variant is administered to the subject multiple times. In some embodiments, a vector or polynucleotide that encodes a wild-type Aβ peptide or an Aβ peptide variant is administered to the subject a single time. Multiple treatments may or may not have the same formulations and/or routes of administration(s).

In some embodiments, the composition comprising a vector encoding a wild-type Aβ peptide or an Aβ peptide variant or a fragment or functional derivative thereof is delivered after onset of Aβ peptide oligomer, protofibril, or fibril formation. In some embodiments, the composition comprising a vector encoding a wild-type Aβ peptide or an Aβ peptide variant or a fragment or functional derivative thereof is delivered after onset of amyloid plaque formation. In some embodiments, the composition comprising a vector encoding a wild-type Aβ peptide or an Aβ peptide variant or a fragment or functional derivative thereof is delivered before onset of Aβ peptide oligomer, protofibril, or fibril formation. In some embodiments, the composition comprising a vector encoding a wild-type Aβ peptide or an Aβ peptide variant or a fragment or functional derivative thereof is delivered before onset of amyloid plaque formation.

A. Carriers

In some embodiments, pharmaceutical compositions of the present disclosure comprise an effective amount of one or more compositions comprising wild-type Aβ peptide or Aβ peptide variants or polynucleotides that encode the variants dissolved or dispersed in a pharmaceutically acceptable carrier. The phrases “pharmaceutical” and “pharmacologically acceptable” and used interchangeably herein refer to molecular entities and compositions that do not produce an adverse, allergic or other untoward reaction when administered to a subject, such as, for example, a human, as appropriate, and do not interfere with the therapeutic methods of the disclosure. The preparation of a pharmaceutical composition that contains at least one vector encoding an Aβ peptide variant or a fragment or functional derivative thereof or additional active ingredient will be known to those of skill in the art in light of the present disclosure, as exemplified by Remington: The Science and Practice of Pharmacy, 21st Ed. Lippincott Williams and Wilkins, 2005, specifically incorporated by reference herein in its entirety. Moreover, for administration to a subject, it will be understood that preparations should meet sterility, pyrogenicity, general safety, and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, specifically incorporated by reference herein in its entirety). Except insofar as any conventional carrier is incompatible with the active ingredient, its use in the pharmaceutical compositions is contemplated. The compositions comprising wild-type Aβ peptide or Aβ peptide variants or polynucleotides encoding such may comprise different types of carriers depending on whether it is to be administered in solid, liquid or aerosol form, and whether it needs to be sterile for such routes of administration, such as injection.

Further in accordance with the present disclosure, the composition of the present disclosure suitable for administration may be provided in a pharmaceutically acceptable carrier with or without an inert diluent. The carrier should be assimilable and includes liquid, semi-solid, i.e., pastes, or solid carriers. Except insofar as any conventional media, agent, diluent or carrier is detrimental to the recipient or to the therapeutic effectiveness of a composition contained therein, its use in practicing the methods of the present disclosure is appropriate. Examples of carriers or diluents include fats, oils, water, saline solutions, lipids, liposomes, resins, binders, fillers, alcohols, and the like, or combinations thereof. The composition may also comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

In accordance with the present disclosure, the composition is combined with the carrier in any convenient and practical manner, i.e., by solution, suspension, emulsification, admixture, encapsulation, absorption and the like. Such procedures are routine for those skilled in the art. The compositions comprising wild-type Aβ peptide or Aβ peptide variants or polynucleotides encoding such may be lyophilized.

In a specific embodiment of the present disclosure, the composition is combined or mixed thoroughly with a semi-solid or solid carrier. The mixing can be carried out in any convenient manner such as grinding. Stabilizing agents can be also added in the mixing process in order to protect the composition from loss of therapeutic activity, i.e., denaturation in the stomach. Examples of stabilizers for use in the composition include buffers, amino acids such as glycine and lysine, carbohydrates such as dextrose, mannose, galactose, fructose, lactose, sucrose, maltose, sorbitol, mannitol, etc.

In further embodiments, the present disclosure may include the use of a pharmaceutical lipid vehicle compositions that incorporate compositions comprising wild-type Aβ peptide or Aβ peptide variants or polynucleotides encoding such, one or more lipids, and an aqueous solvent. As used herein, the term “lipid” will be defined to include any of a broad range of substances that is characteristically insoluble in water and extractable with an organic solvent. This broad class of compounds is well known to those of skill in the art, and as the term “lipid” is used herein, it is not limited to any particular structure. Examples include compounds which contain long-chain aliphatic hydrocarbons and their derivatives. A lipid may be naturally occurring or synthetic (i.e., designed or produced by man). However, a lipid is usually a biological substance. Biological lipids are well known in the art, and include for example, neutral fats, phospholipids, phosphoglycerides, steroids, terpenes, lysolipids, glycosphingolipids, glycolipids, sulphatides, lipids with ether and ester-linked fatty acids and polymerizable lipids, and combinations thereof. Of course, compounds other than those specifically described herein that are understood by one of skill in the art as lipids are also encompassed by the compositions and methods of the present disclosure.

One of ordinary skill in the art would be familiar with the range of techniques that can be employed for dispersing a composition in a lipid vehicle. For example, the composition(s) comprising wild-type Aβ peptide or Aβ peptide variants or polynucleotides encoding such may be dispersed in a solution containing a lipid, dissolved with a lipid, emulsified with a lipid, mixed with a lipid, combined with a lipid, covalently bonded to a lipid, contained as a suspension in a lipid, contained or complexed with a micelle or liposome, or otherwise associated with a lipid or lipid structure by any means known to those of ordinary skill in the art. The dispersion may or may not result in the formation of liposomes.

The composition(s) comprising a vector encoding a wild-type Aβ peptide or an Aβ peptide variant or a fragment or functional derivative thereof may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine. Upon formulation, solutions will be administered in a manner compatible with the dosage formulation and in such amount as is therapeutically effective. The formulations are easily administered in a variety of dosage forms such as formulated for parenteral administrations such as injectable solutions, or aerosols for delivery to the lungs, or formulated for alimentary administrations such as drug release capsules and the like.

B. Routes of Administration

The therapeutic agents of the disclosure may be administered by the same route of administration or by different routes of administration. The route of administration of the composition may be, for example, intravenously, intracerebrally, intracranially, intramuscularly, subcutaneously, topically, orally, mucosally, intradermally, transdermally, intraperitoneally, intraarterially, intraorbitally, by implantation, intravaginally, intrarectally, intrathecally, intraarticularly, intraventricularly, intrasynovially, or intranasally; by inhalation, injection, infusion, continuous infusion, localized perfusion bathing target cells directly, via a catheter, via a lavage; in creams or in lipid compositions (e.g., liposomes); by other method or any combination of the forgoing as would be known to one of ordinary skill in the art (see, for example, Remington's Pharmaceutical Sciences, 18th Ed. Mack Printing Company, 1990, specifically incorporated by reference herein in its entirety).

In some embodiments, the composition(s) comprising a vector encoding a wild-type Aβ peptide or an Aβ peptide variant or a fragment or functional derivative thereof is delivered systemically or locally. In some embodiments, the composition comprising a vector encoding a wild-type Aβ peptide or an Aβ peptide variant or a fragment or functional derivative thereof is delivered to the central nervous system via retro-orbital injection. In some embodiments, the composition comprising a vector encoding a wild-type Aβ peptide or an Aβ peptide variant or a fragment or functional derivative thereof is delivered to the central nervous system systemically via peripheral injection. In some embodiments, the peripheral injection is intravenous injection. In some embodiments, the composition comprising a vector encoding a wild-type Aβ peptide or an Aβ peptide variant or a fragment or functional derivative thereof is delivered to cerebrospinal fluid (CSF In some embodiments, the composition is delivered to the CSF by nonsurgical injection. In some embodiments, nonsurgical injection into the CSF comprises nonsurgical intrathecal injection. In some embodiments, the composition is delivered to the CSF by neurosurgical injection. In some embodiments, neurosurgical injection into the CSF comprises neurosurgical injection into the cisterna magna. In some embodiments, the composition comprising a vector encoding a wild-type Aβ peptide or an Aβ peptide variant or a fragment or functional derivative thereof is delivered to the ventricular system. In some embodiments, the composition is delivered to the ventricular system by neurosurgical injection. Delivery of a composition to the CSF and/or the ventricular system is described in, for example, W. A. Liguore et al., Molecular Therapy 27(11):2018-2037, incorporated by reference herein in its entirety. In some embodiments, the composition delivered to the central nervous system, the CSF, and/or the ventricular system crosses the blood brain barrier.

In some embodiments, compositions comprising wild-type Aβ peptide or Aβ peptide variants disclosed herein may be formulated so as to enhance the stability of the wild-type Aβ peptide or Aβ peptide variants in vivo and/or uptake or absorption of the wild-type Aβ peptide or Aβ peptide variants by cells, as explained in A. L. Lewis and J. Richard, Therapeutic Delivery 6(2):149-163 (2015), specifically incorporated by reference herein in its entirety. For example, wild-type Aβ peptide or Aβ peptide variants may be formulated with an absorption enhancer, e.g., acyl carnitine, sodium octanoate, sodium caprate, SNAC, SNAD, 5-CNAC, to increase absorption of the wild-type Aβ peptide or Aβ peptide variants by cells. In some embodiments, formulations to enhance the stability of wild-type Aβ peptide or Aβ peptide variants in vivo and/or uptake or absorption of wild-type Aβ peptide or Aβ peptide variants by cells will depend on the route of administration of the compositions, for example, orally or by injection.

1. Parenteral Routes

Thus, in some embodiments, the composition(s) comprising a vector encoding a wild-type Aβ peptide or an Aβ peptide variant or a fragment or functional derivative thereof may be administered via a parenteral route. As used herein, the term “parenteral” includes routes that bypass the alimentary tract. Specifically, the pharmaceutical compositions disclosed herein may be administered for example, but not limited to retro-orbitally, intracerebrally, intracranially, intravenously, intradermally, intramuscularly, intraarterially, intrathecally, subcutaneous, or intraperitoneally U.S. Pat. Nos. 6,7537,514, 6,613,308, 5,466,468, 5,543,158; 5,641,515; and 5,399,363 (each specifically incorporated herein by reference in its entirety).

Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (see, e.g., U.S. Pat. No. 5,466,468, specifically incorporated herein by reference in its entirety). In all cases the form must be sterile and must be fluid to the extent that easy injectability exists. It must be stable under the conditions of manufacture and storage and must be preserved against the contaminating action of microorganisms, such as bacteria and fungi. The carrier can be a solvent or dispersion medium containing, for example, water, ethanol, polyol (i.e., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed will be known to those of skill in the art in light of the present disclosure. For example, one dosage may be dissolved in isotonic NaCl solution and injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Sterile injectable solutions may be prepared by incorporating the active compounds in the required amount in the appropriate solvent with various of the other ingredients enumerated above, as required, followed by filtered sterilization, for example. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. A powdered composition is combined with a liquid carrier such as, e.g., water or a saline solution, with or without a stabilizing agent.

2. Alimentary Routes

In particular embodiments of the present disclosure, the composition(s) comprising a vector encoding a wild-type Aβ peptide or an Aβ peptide variant or a fragment or functional derivative thereof is formulated to be administered via an alimentary route. Alimentary routes include all possible routes of administration in which the composition is in direct contact with the alimentary tract. Specifically, the pharmaceutical compositions disclosed herein may be administered orally, buccally, rectally, or sublingually. As such, these compositions may be formulated with an inert diluent or with an assimilable edible carrier, or they may be enclosed in hard- or soft-shell gelatin capsule, or they may be compressed into tablets, or they may be incorporated directly with the food of the diet.

In certain embodiments, the active compounds may be incorporated with excipients and used in the form of ingestible tablets, buccal tables, troches, capsules, elixirs, suspensions, syrups, wafers, and the like (Mathiowitz et al., 1997; Hwang et al., 1998; U.S. Pat. Nos. 5,641,515; 5,580,579 and 5,792,451, each specifically incorporated herein by reference in its entirety). The tablets, troches, pills, capsules and the like may also contain the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. When the dosage form is a capsule, it may contain, in addition to materials of the above type, carriers such as a liquid carrier. Gelatin capsules, tablets, or pills may be enterically coated. Enteric coatings prevent denaturation of the composition in the stomach or upper bowel where the pH is acidic. See, e.g., U.S. Pat. No. 5,629,001. Upon reaching the small intestines, the basic pH therein dissolves the coating and permits the composition to be released and absorbed by specialized cells, e.g., epithelial enterocytes and Peyer's patch M cells. A syrup of elixir may contain the active compound sucrose as a sweetening agent methyl and propylparabens as preservatives, a dye and flavoring, such as cherry or orange flavor. Of course, any material used in preparing any dosage unit form should be pharmaceutically pure and substantially non-toxic in the amounts employed. In addition, the active compounds may be incorporated into sustained-release preparation and formulations.

For oral administration, the composition(s) comprising a vector encoding a wild-type Aβ peptide or an Aβ peptide variant or a fragment or functional derivative thereof of the present disclosure may alternatively be incorporated with one or more excipients in the form of a mouthwash, dentifrice, buccal tablet, oral spray, or sublingual orally-administered formulation. For example, a mouthwash may be prepared incorporating the active ingredient in the required amount in an appropriate solvent, such as a sodium borate solution (Dobell's Solution). Alternatively, the active ingredient may be incorporated into an oral solution such as one containing sodium borate, glycerin and potassium bicarbonate, or dispersed in a dentifrice, or added in a therapeutically-effective amount to a composition that may include water, binders, abrasives, flavoring agents, foaming agents, and humectants. Alternatively, the compositions may be fashioned into a tablet or solution form that may be placed under the tongue or otherwise dissolved in the mouth.

Additional formulations which are suitable for other modes of alimentary administration include suppositories. Suppositories are solid dosage forms of various weights and shapes, usually medicated, for insertion into the rectum. After insertion, suppositories soften, melt or dissolve in the cavity fluids. In general, for suppositories, traditional carriers may include, for example, polyalkylene glycols, triglycerides or combinations thereof. In certain embodiments, suppositories may be formed from mixtures containing, for example, the active ingredient in the range of about 0.5% to about 10% (by weight), and preferably about 1% to about 2% (by weight).

3. Miscellaneous Routes

In other embodiments of the disclosure, the composition(s) comprising a vector encoding a wild-type Aβ peptide or an Aβ peptide variant or a fragment or functional derivative thereof may be formulated for administration via various miscellaneous routes, for example, topical (i.e., transdermal) administration, mucosal administration (intranasal, vaginal, etc.) and/or inhalation.

Pharmaceutical compositions for topical administration may include the active compound formulated for a medicated application such as an ointment, paste, cream or powder. Ointments include all oleaginous, adsorption, emulsion and water-soluble based compositions for topical application, while creams and lotions are those compositions that include an emulsion base only. Topically administered medications may contain a penetration enhancer to facilitate adsorption of the active ingredients through the skin. Suitable penetration enhancers include glycerin, alcohols, alkyl methyl sulfoxides, pyrrolidones and luarocapram. Possible bases for compositions for topical application include polyethylene glycol, lanolin, cold cream and petrolatum as well as any other suitable absorption, emulsion or water-soluble ointment base. Topical preparations may also include emulsifiers, gelling agents, and antimicrobial preservatives as necessary to preserve the active ingredient and provide for a homogenous mixture. Transdermal administration of the present disclosure may also comprise the use of a “patch”. For example, the patch may supply one or more active substances at a predetermined rate and in a continuous manner over a fixed period of time.

In certain embodiments, the pharmaceutical composition(s) comprising a vector encoding an Aβ peptide variant or a fragment or functional derivative thereof may be delivered by eye drops, intranasal sprays, inhalation, and/or other aerosol delivery vehicles. Methods for delivering compositions directly to the lungs via nasal aerosol sprays has been described e.g., in U.S. Pat. Nos. 5,756,353 and 5,804,212 (each specifically incorporated herein by reference in its entirety). Likewise, the delivery of drugs using intranasal microparticle resins (see, e.g., Takenaga et al., 1998) and lysophosphatidyl-glycerol compounds (see, e.g., U.S. Pat. No. 5,725,871, specifically incorporated herein by reference in its entirety) are also well-known in the pharmaceutical arts. Likewise, transmucosal drug delivery in the form of a polytetrafluoroetheylene support matrix is described in, e.g., U.S. Pat. No. 5,780,045 (specifically incorporated herein by reference in its entirety).

The term aerosol refers to a colloidal system of finely divided solid of liquid particles dispersed in a liquefied or pressurized gas propellant. The typical aerosol of the present disclosure for inhalation will consist of a suspension of active ingredients in liquid propellant or a mixture of liquid propellant and a suitable solvent. Suitable propellants include hydrocarbons and hydrocarbon ethers. Suitable containers will vary according to the pressure requirements of the propellant. Administration of the aerosol will vary according to subject's age, weight and the severity and response of the symptoms.

C. Dosing

The appropriate dosage amount of a composition(s) of the present disclosure administered to the subject can be determined by physical and physiological factors such as body weight, severity and course of condition, the type of disease being treated, the clinical condition of the individual, previous or concurrent therapeutic interventions, the individual's clinical history and response to the treatment, idiopathy of the subject, the route of administration, and the discretion of the attending physician. Depending upon the dosage and the route of administration, the number of administrations of a preferred dosage and/or an effective amount may vary according to the response of the subject. The practitioner responsible for administration will, in any event, determine the concentration of active ingredient(s) in a composition and appropriate dose(s) for the individual subject.

In certain embodiments, pharmaceutical compositions may comprise, for example, at most or least about 0.000001 to at most or at least about 10% (by weight) of an active compound. In other embodiments, the active compound may comprise between about 0.001% to about 1% of the weight of the unit, or about 0.01% to about 0.1%, for example, and any range derivable therein. Naturally, the amount of active compound(s) in each therapeutically useful composition may be prepared in such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some embodiments, a unit dose comprises a single administrable dose.

The quantity to be administered, both according to number of treatments and unit dose, depends on the treatment effect desired. An effective dose is understood to refer to an amount necessary to achieve a particular effect. Furthermore, such doses can be administered at multiple times during a day, and/or on multiple days, weeks, or months.

In some embodiments, a single dose of a wild-type Aβ peptide or an Aβ peptide variant is administered. In some embodiments, multiple doses of an Aβ peptide variant are administered. In some embodiments, an effective dose of a wild-type Aβ peptide or an Aβ peptide variant is administered. In some embodiments, a wild-type Aβ peptide or an A peptide variant is administered at a dose of at least, at most, or about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range or value derivable therein. In certain embodiments, the effective dose of an Aβ peptide variant is one which can provide a blood level of about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μM or any range derivable therein. In certain embodiments, a wild-type Aβ peptide or an Aβ peptide variant that is administered to a subject is metabolized in the body to a metabolized therapeutic agent, in which case the blood levels may refer to the amount of that agent. Alternatively, to the extent a wild-type Aβ peptide or an Aβ peptide variant is not metabolized by a subject, the blood levels discussed herein may refer to the unmetabolized wild-type or variant Aβ peptide.

In some embodiments, a single dose of a polynucleotide that encodes a wild-type Aβ peptide or an Aβ peptide variant is administered. In some embodiments, multiple doses of the polynucleotide that encodes a wild-type Aβ peptide or an Aβ peptide variant are administered. In some embodiments, an effective dose of the polynucleotide that encodes a wild-type Aβ peptide or an Aβ peptide variant is administered. In some embodiments, the polynucleotide that encodes a wild-type Aβ peptide or an Aβ peptide variant is administered at a dose of at least, at most, or about 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, or 1×10¹⁸ polynucleotide copies/kg body weight of the subject, or any range or value derivable therein. In some embodiments, the polynucleotide that encodes a wild-type Aβ peptide or an Aβ peptide variant is administered at a dose of between 1×10⁸ to 1×10¹⁸ polynucleotide copies/kg body weight of the subject. In some embodiments, the polynucleotide that encodes a wild-type Aβ peptide or an Aβ peptide variant is administered at a dose of between 1×10¹¹ to 1×10¹⁴ polynucleotide copies/kg body weight of the subject. In some embodiments, the polynucleotide that encodes a wild-type Aβ peptide or an Aβ peptide variant is administered at a dose of between 1×10¹² to 1×10¹⁵ polynucleotide copies/kg body weight of the subject.

In some embodiments, the polynucleotide that encodes a wild-type Aβ peptide or an Aβ peptide variant is comprised in a vector. In some embodiments, the polynucleotide that encodes a wild-type Aβ peptide or an Aβ peptide variant is comprised in a vector encoding a minigene that enables protein expression of variant Aβ peptides. In some embodiments, an effective dose of the vector comprising the polynucleotide that encodes a wild-type Aβ peptide or an Aβ peptide variant is administered. In some embodiments, the vector is administered at a dose of at least, at most, or about 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, or 1×10¹⁸ vector genomes/kg body weight of the subject, or any range or value derivable therein. In some embodiments, the vector is administered at a dose of between 1×10⁸ to 1×10¹⁸ vector genomes/kg body weight of the subject. In some embodiments, the vector is administered at a dose of between 1×10¹¹ to 1×10¹⁴ vector genomes/kg body weight of the subject. In some embodiments, the vector is administered at a dose of between 1×10¹² to 1×10¹⁵ vector genomes/kg body weight of the subject.

In some embodiments, a single dose of one or more additional neurodegenerative disease medications is administered. In some embodiments, multiple doses of the one or more additional neurodegenerative disease medications are administered. In some embodiments, an effective dose of the one or more additional neurodegenerative disease medications is administered. In some embodiments, the one or more additional neurodegenerative disease medications are administered at a dose of at least, at most, or about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range or value derivable therein. In certain embodiments, the effective dose of the one or more additional neurodegenerative disease medications is one which can provide a blood level of about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μM or any range derivable therein. In certain embodiments, the one or more additional neurodegenerative disease medications that is administered to a subject is metabolized in the body to a metabolized therapeutic agent, in which case the blood levels may refer to the amount of that agent. Alternatively, to the extent the one or more additional neurodegenerative disease medications is not metabolized by a subject, the blood levels discussed herein may refer to the unmetabolized wild-type or variant Aβ peptide.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/ml or mM (blood levels). It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

### V. Kits

Certain aspects of the present disclosure also concern kits containing compositions of the disclosure or compositions to implement methods of the disclosure. In some embodiments, kits can be used to evaluate one or more biomarkers (e.g., Aβ peptide oligomers, protofibrils, fibrils, plaques, misfolded proteins, tau protein, phosphorylated tau protein), seeded tau protein and/or seeded Aβ peptide, neuroinflammatory markers, markers of cognitive decline, neurodegenerative markers, marks of neuronal loss, and/or markers of synaptic loss). In certain embodiments, kits can be used to measure Aβ peptide or other protein expression in vitro or in vivo. In certain embodiments, a kit contains, contains at least or contains at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 100, 500, 1,000 or more probes, primers or primer sets, synthetic molecules or inhibitors, or any value or range and combination derivable therein. In some embodiments, there are kits for evaluating biomarker activity in a cell.

Kits may comprise components, which may be individually packaged or placed in a container, such as a tube, bottle, vial, syringe, or other suitable container means.

Individual components may also be provided in a kit in concentrated amounts; in some embodiments, a component is provided individually in the same concentration as it would be in a solution with other components. Concentrations of components may be provided as 1×, 2×, 5×, 10×, or 20× or more.

Kits for using probes, synthetic nucleic acids, nonsynthetic nucleic acids, synthetic peptides, nonsynthetic peptides, and/or inhibitors of the disclosure for prognostic or diagnostic applications are included as part of the disclosure. Specifically contemplated are any such molecules corresponding to any biomarker identified herein, which includes nucleic acid primers/primer sets and probes that are identical to or complementary to all or part of a biomarker, which may include noncoding sequences of the biomarker, as well as coding sequences of the biomarker.

In certain aspects, negative and/or positive control nucleic acids, probes, and inhibitors are included in some kit embodiments. In addition, a kit may include a sample that is a negative or positive control for methylation of one or more biomarkers.

Any embodiment of the disclosure involving specific biomarker by name is contemplated also to cover embodiments involving biomarkers whose sequences are at least 80, 81, 82, 83, 84, 85, 86, 87, 88, 89, 90, 91, 92, 93, 94, 95, 96, 97, 98, 99% identical to the mature sequence of the specified nucleic acid.

It is contemplated that any method or composition described herein can be implemented with respect to any other method or composition described herein and that different embodiments may be combined. The claims originally filed are contemplated to cover claims that are multiply dependent on any filed claim or combination of filed claims.

### EXAMPLES

The following examples are included to demonstrate embodiments of the invention. It should be appreciated by those of skill in the art that the techniques disclosed in the examples which follow represent techniques discovered by the inventor to function well in the practice of the invention. However, those of skill in the art should, in light of the present disclosure, appreciate that many changes can be made in the specific embodiments which are disclosed and still obtain a like or similar result without departing from the spirit and scope of the invention.

### Example 1—Gene Therapy Using Aβ Variants for Amyloid Reduction

Peptide inhibitors which met four therapeutic criteria were identified in vitro, and the top two peptide candidates were vetted in vivo in an animal model of Aβ amyloidosis. A minigene vector was created to express variant Aβ peptide at the cell membrane where its release into the extracellular space would be regulated by endogenous proteases. AAV was then used to deliver the vector into the neonatal mouse brain for widespread, lifelong neuronal expression to test efficacy for Aβ reduction in the APP/PS1 mouse model of AD. This work yielded the counterintuitive finding that peptides differing from pathogenic Aβ by just one or two amino acids effectively diminished Aβ fibrillization, destabilized existing fibrils, and lowered oligomer toxicity over short periods in vitro, while reducing amyloid aggregation and neuroinflammation when used chronically in vivo.

This work also takes the first steps in applying viral-mediated gene therapy to overcome past limitations on advancing peptide Aβ inhibitors from the bench to the brain. The broader impact of the work lies in the potential for applying this strategy to other protein misfolding diseases involving the aggregation of misfolded proteins into toxic species that provoke neurodegeneration where peptide treatments have been eschewed for technical reasons that can now be overcome through expression engineering and viral technology.

A. F20P and F19D/L34P Variants Inhibit Aggregation of Wild-Type Aβ42

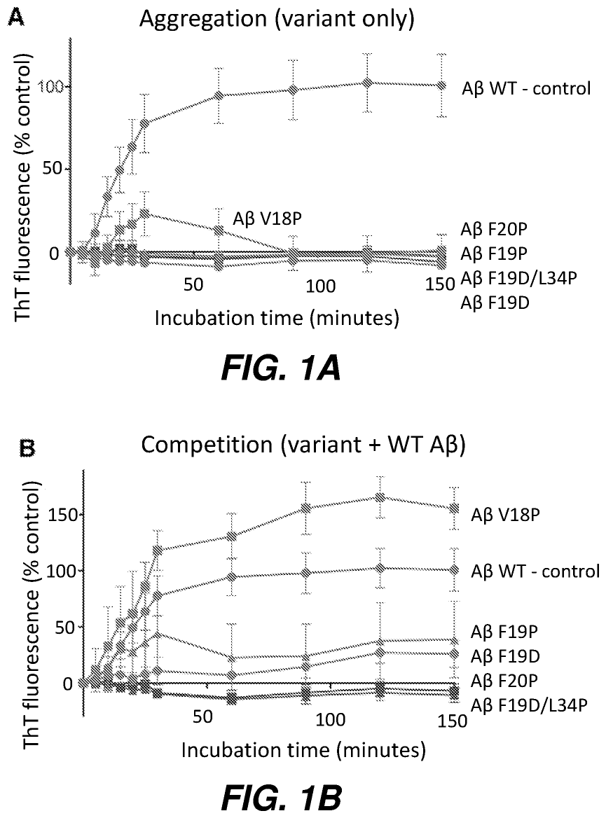

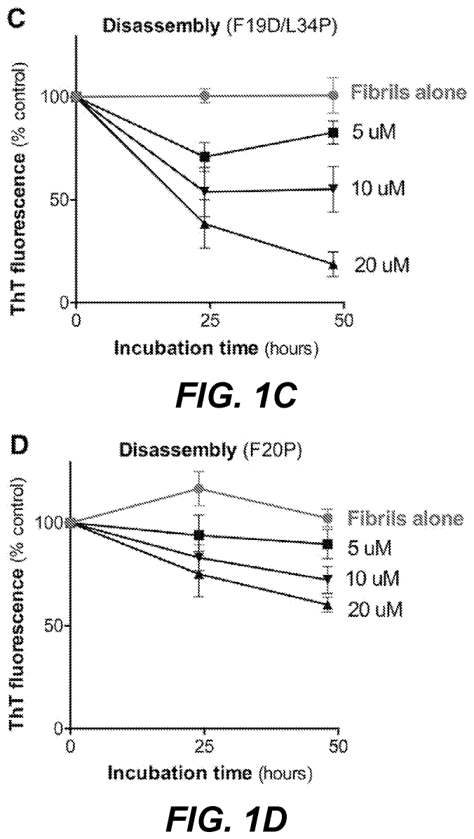

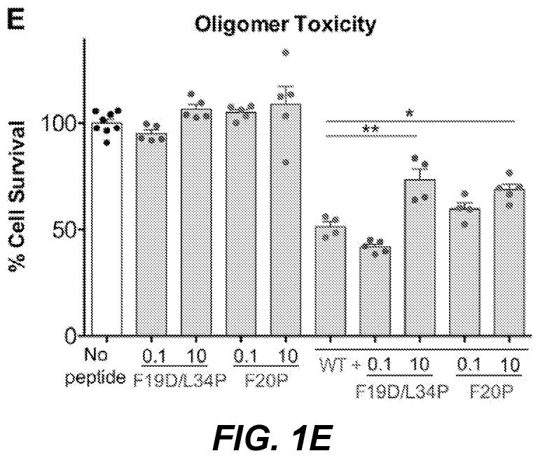

The central hydrophobic region of Aβ (17Leu-Val-Phe-Phe-Ala21) governs the rate of monomeric assembly and forms the β-sheet hairpin in mature fibrils. Given its importance in fibril formation, in some embodiments, targeted amino acid substitution in this region yields peptides that could prevent aggregation of wild-type Aβ42 peptide. Based on past studies of this domain, 5 different Aβ42 variant peptides were examined: V18P, F19D, F19P, F19D/L34P, and F20P. It was first confirmed using Thioflavin-T assay that none of the five variants self-aggregated into fibrils (FIG. 1A). It was next tested whether any of the five peptides could competitively inhibit the fibrillization of wild-type Aβ42 when both peptides were mixed at equimolar ratios prior to starting the fibrillization reaction (FIG. 1B). Four of the five variant peptides reduced fibrillization of wild-type Aβ, while one (V18P) exacerbated aggregation. Two peptide variants selected for further study, F20P and F19D/L34P, appeared to completely prevent aggregation. These two peptides were tested for the potential to disassemble wild-type Aβ42 when added after fibril formation. Both variants disaggregated wild-type fibrils in a concentration-dependent manner, however, F19D/L34P was considerably more effective at fibril disassembly than F20P, attaining >80% loss of ThT signal at the highest concentration tested (FIGS. 1C, 1D).

Finally, the potential for variant Aβ peptides to abate cytotoxicity caused by wild-type Aβ oligomers was examined. For this experiment, wild-type Aβ42 was used alone or mixed at an equimolar ratio with variant peptide prior to oligomer assembly. Twenty-four hours later, peptide solutions were added to the media of the murine neuroblastoma 2a cell line (N2a) cell cultures. Oligomers assembled from wild-type Aβ42 caused nearly 50% cell loss compared to cultures treated with oligomerization buffer alone (FIG. 1E). In contrast, neither F20P nor F19D/L34P significantly altered cell viability on its own. Notably, cell survival increased from 50% of control with wild-type Aβ42 alone to approximately 70% when wild-type peptide was co-incubated with either variant during oligomer assembly. Thus, in some embodiments, the F20P and F19D/L34P variants meet the minimum in vitro criteria for candidate aggregation inhibitors.

B. A Vector Design to Secrete Aβ Variants from Mammalian Cells

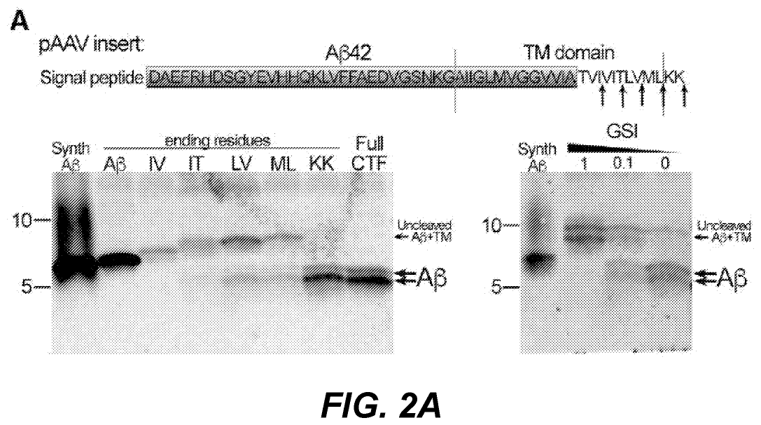

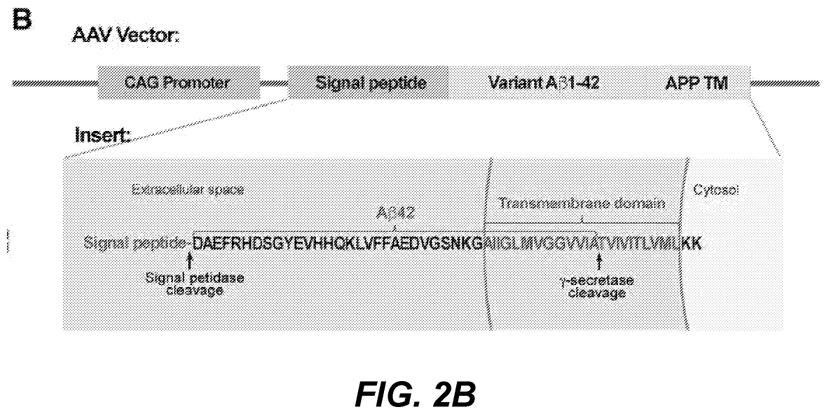

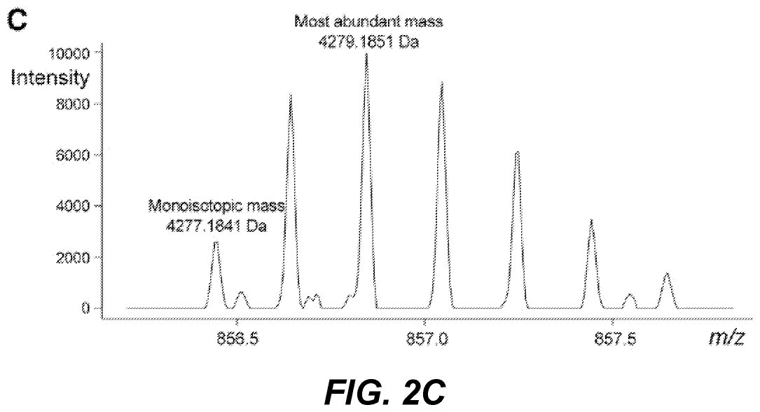

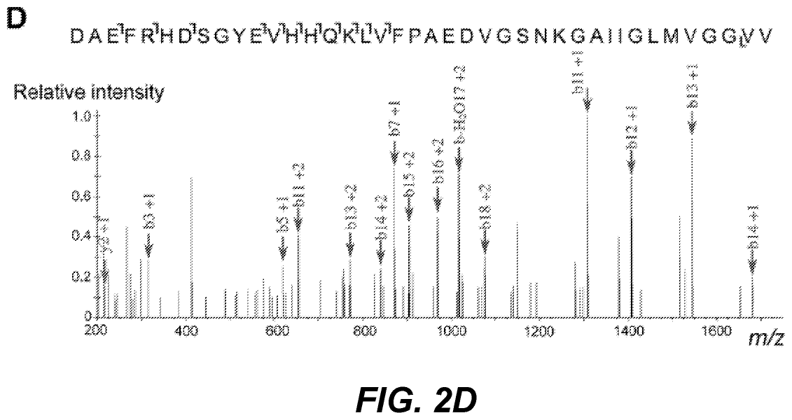

The Aβ peptide variants were next tested in the brain at levels high enough to be therapeutically effective. An expression strategy was designed to stably express variant Aβ at the cell membrane where its release into the extracellular space would be regulated by endogenous γ-secretase. Previous studies achieved this goal by fusing the signal peptide from amyloid precursor protein (APP) to the APP C-terminal fragment (β-CTF), however, while this construct did produce extracellular Aβ, R-CTF overexpression also unexpectedly caused Aβ-independent lysosomal-autophagic pathology. The inventors therefore sought to avoid potential lysosomal complications by using the smallest R-CTF fragment needed for 7-secretase cleavage. The Gaussia luciferase signal peptide was used to target the membrane, and this signal peptide was fused to a series of R-CTF fragments. The longest fragment was 54 amino acids in length and included Aβ42 and the entire APP transmembrane domain plus two intracellular lysine residues (FIG. 2A). Four additional constructs were each truncated by 2 amino acids into the transmembrane domain; the shortest construct contained just 45 residues (Aβ42+3 additional).

The different constructs were transfected into N2a cells and secreted Aβ measured in the conditioned cell culture medium using immunoprecipitation followed by immunoblot. The complete APP transmembrane domain plus 2 intracellular lysines was found to be both necessary and sufficient to achieve Aβ secretion at levels equivalent to those observed using a construct encoding the full-length CTF (FIG. 2A). Addition of the γ-secretase inhibitor LY411575 blocked release of variant Aβ into the media in a concentration-dependent manner, confirming that Aβ secretion was dependent on γ-secretase activity (FIG. 2A).

This minimal expression construct was cloned into an AAV delivery vector for subsequent in vivo expression under control of the ubiquitous CAG promoter (FIG. 2B). The woodchuck poliovirus response element was included at the 3′ end to stabilize the mRNA and followed by a bovine growth hormone poly A sequence. Finally, mass spectrometry was used to confirm that this vector truly produced Aβ40/42 when expressed in cells. N2a cells were transfected with the viral Aβ F20P vector and then human Aβ was immunoprecipitated from the media as for the WT peptide. Liquid chromatography-tandem mass spectrometry (LC-MS/MS) was performed without enzymatic digestion to determine whether a full-length peptide was made. This analysis confirmed the presence of peptides with the appropriate mass for Aβ40 F20P (FIG. 2C) and Aβ42 F20P (data not shown), along with the correct sequence for each peptide (FIG. 2D).

C. Using AAV to Express Aβ Variants In Vivo

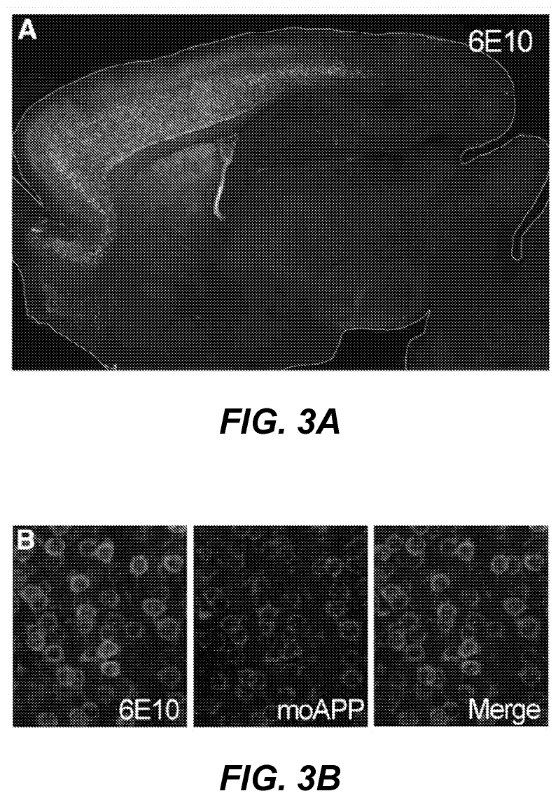

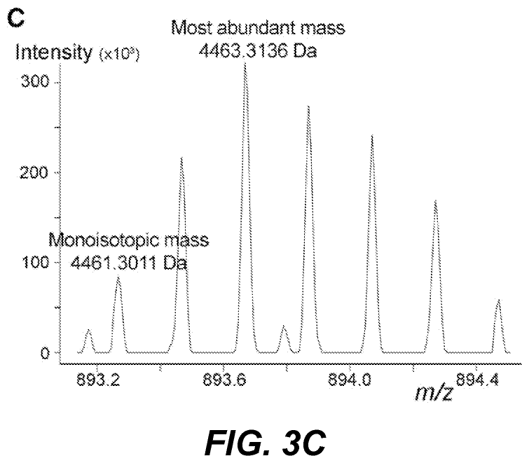

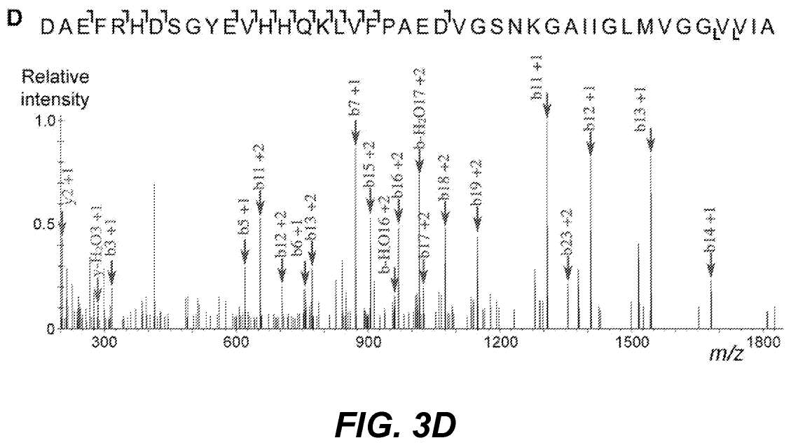

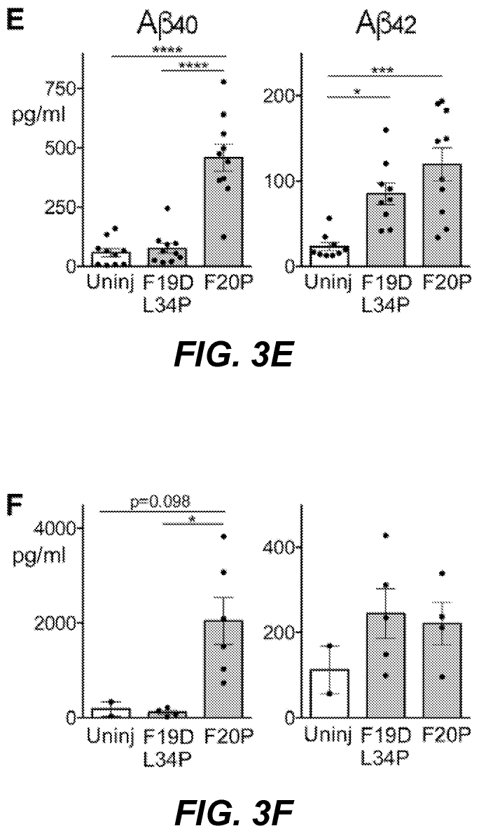

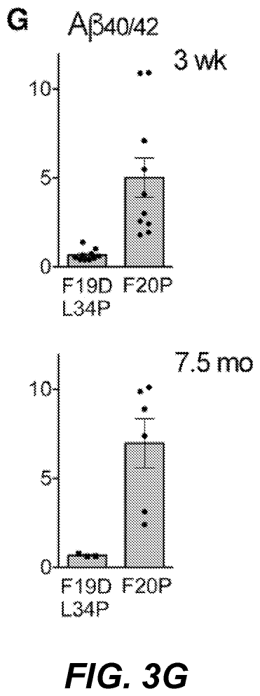

Wild-type mice were injected to confirm that the peptides would be expressed and secreted in vivo. Both Aβ F19D/L34P and F20P expression constructs were packaged into AAV8 and injected into the lateral ventricles of neonatal (P0) mice. Mice were euthanized 3-4 weeks after injection to examine expression levels and viral distribution. Viral spread was assessed by immunostaining using human-specific APP antibody (6E10) to detect variant Aβ. Viral expression was biased to the frontal cortex and so subsequent analyses focused on this region (FIG. 3A). Co-immunostaining with 6E10 to detect variant Aβ and Y188 to detect endogenous mouse APP confirmed that the virally-delivered precursor peptide was located at the cell membrane alongside endogenous APP (FIG. 3B).

IP followed by MS was used for homogenates from WT mice transduced with Aβ F20P to test for expression of full-length peptide in vivo. Both Aβ40 and Aβ-42 F20P from brain homogenate displayed elution times identical to Aβ isolated from cell media; both peptides were again identified at the expected mass and sequence confirmed (FIGS. 3C, 3D; data shown for Aβ42 F20P). Notably, none of the partial cleavage products, which could have been immunoprecipitated with 6E10 (such as the intact TM domain without signal peptide or γ-secretase-cleaved Aβ still attached to the signal peptide), appeared in the spectra.

In vivo production of each variant Aβ peptide was also measured by ELISA. The human-specific capture antibody and end-specific detection antibodies of this assay ensured that the peptides measured were 1) human, and therefore virally-delivered variant Aβ, and 2) mature Aβ that had undergone γ-secretase cleavage. F20P expressed well in vivo. Similar to native APP, this construct produced Aβx-40 peptide at levels several fold greater than Aβx-42 (FIG. 3E). In contrast, F19D/L34P produced high levels of Aβx-42 but no detectable Aβx-40, indicating that, in some embodiments, the L34P substitution disrupts end-specific Aβ40 antibody binding. These results suggest that, in some embodiments, viral transduction with the minimal expression vector achieves cell membrane localization and γ-secretase cleavage, releasing variant Aβ peptide into the brain.

D. Virally-Delivered Variant Peptides Reduce Aβ Load in APP/PS1 Transgenic Mice

Virus encoding Aβ F20P or F19D/L34P was injected into neonatal (P0) APP/PS1 mice and non-transgenic littermates. Animals were harvested at 7.5 months of age, shortly after the onset of amyloid deposits in this model. Cortical tissue from non-transgenic siblings was used to confirm that virally-expressed Aβ was still produced at this age (FIG. 3F). AAV-injected non-transgenic mice showed the same relative levels of Aβ40:42 as at 3 weeks, with elevated x-42 for both variants and elevated x-40 only detected from F20P (FIG. 3G).

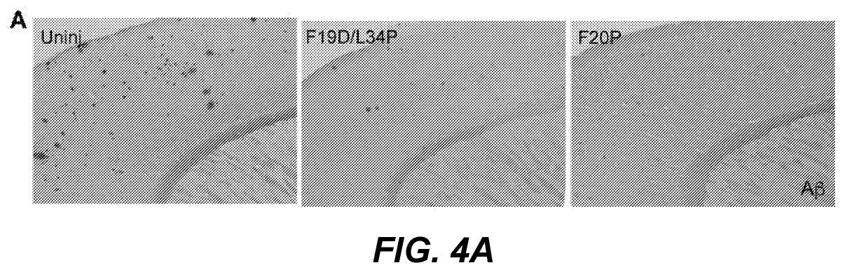

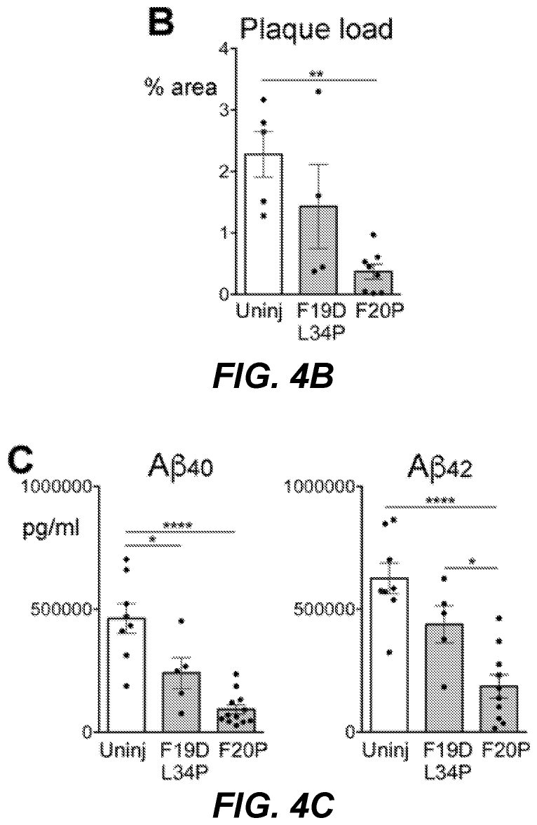

In APP/PS1 animals, plaque burden was assessed by Aβ immunostaining in frontal cortex of one hemisphere and by Aβ ELISA in the other. Compared with uninjected APP/PS1 control mice, mice treated with F20P showed an almost 75% decrease in Aβ deposition (FIGS. 4A, 4B). In agreement with immunostaining, levels of insoluble Aβx-40 and Aβx-42 in mice treated with F20P were also markedly decreased (FIG. 4C). The effects of F19D/L34P expression in APP/PS1 mice were more variable than F20P. Insoluble Aβx-40 levels were significantly reduced in APP/PS1 mice expressing F19D/L34P, however, neither Aβx-42 nor plaque load were significantly diminished (FIGS. 4B, 4C). Thus, while both F20P and F19D/L34P significantly diminished the total accumulation of Aβ, F20P was more effective at inhibiting aggregation in APP/PS1 mice. Neither F20P- nor F19D/L34P-treated non-transgenic animals accumulated detectable levels of human Aβ over 7.5 months of expression (data not shown; total Aβ 40+42, soluble+insoluble, uninjected 100.7±45.5 (n=6); F19D/34P 72.3±23.6 (n=11); F20P 158.2±35.2 μg/ml (n=10); ANOVA F(2, 24)=2.02, p=0.15).

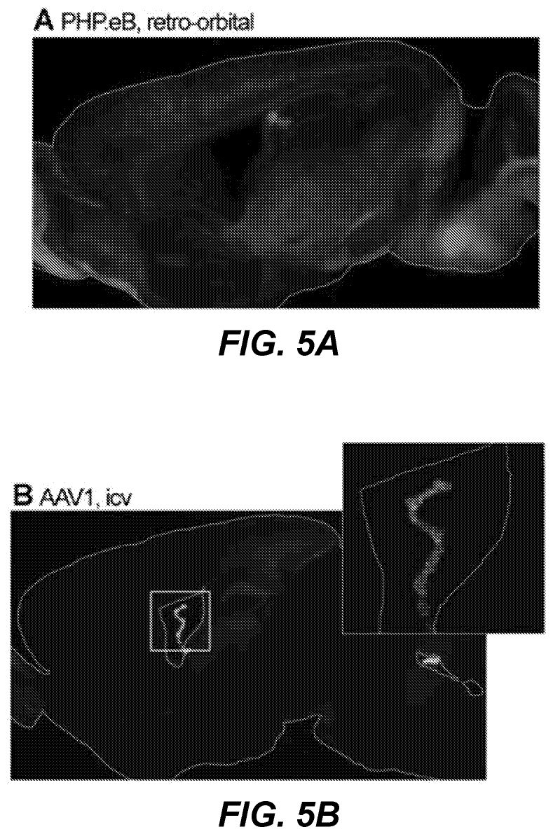

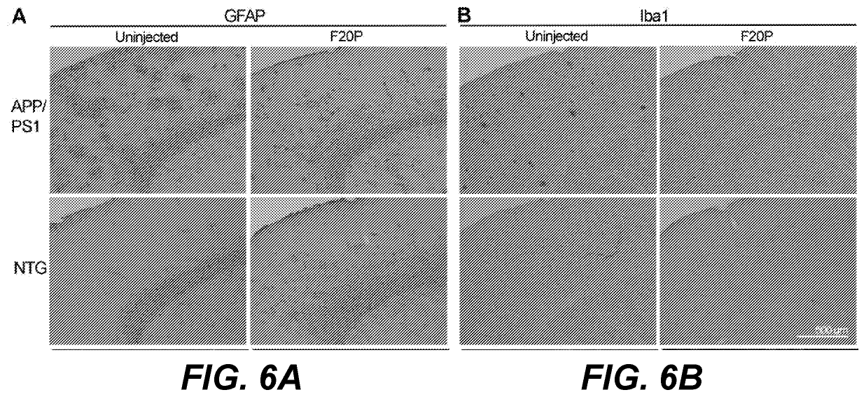

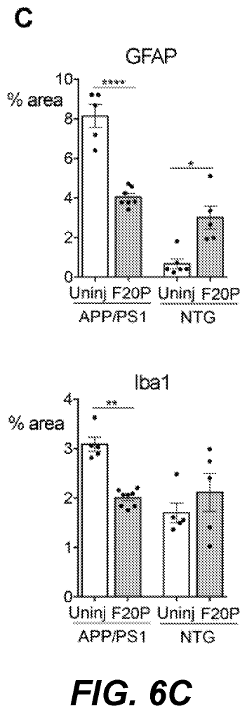

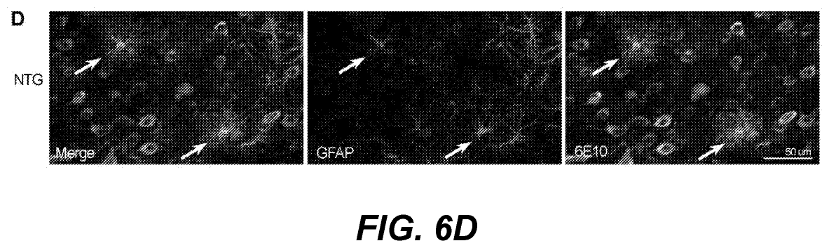

Because the biophysical analysis suggested that at least one of the variants, F19D/L34P, was capable not only of preventing Aβ assembly but also of promoting disassembly of existing Aβ fibrils in vitro (FIG. 1C), it was tested whether the variants can support plaque clearance if administered after amyloid onset. Newly evolved AAV serotypes such as PHP.eB were used to achieve viral delivery and expression in the adult brain, allowing CNS delivery via peripheral injection. Injection of AAV PHP.eB was optimized using fluorescent proteins as a readout for transduction efficiency and spatial distribution across the brain (FIG. 5A). The AAV PHP.eB serotype was found to match the spread and approach the density achieved by neonatal AAV8 injection, suggesting that it will deliver variant Aβ at levels sufficient for use in vivo. Viral transduction also allowed testing of whether alternate routes of administration that may used clinically can match the efficacy of neuronal transduction. Viral transduction of the choroid plexus has been used for gene therapy in mouse models of lysosomal storage disease, AD, and ALS. The inventors found ependymal cells of the choroid plexus can be readily transduced by AAV1, which is used hereinto test the potential for CSF delivery of variant Aβ (FIG. 5B).

E. Variant Aβ Expression Decreases Overall Neuroinflammation in APP/PS1 Mice, but May Induce Mild Astrocytosis

Amyloid plaques elicit a pronounced neuroimmune response in which hypertrophic astrocytes and microglia migrate or divide to surround cored deposits. Under most conditions, the extent of glial induction parallels the severity of amyloid load. Generally speaking, treatments that slow plaque accumulation also temper these changes in glial morphology and localization. Therefore, it was examined whether clustering of hypertrophic astrocytes and microglia that normally delimits cored Aβ deposits decreases alongside plaque load in mice treated with variant Aβ. Analysis was focused on the F20P variant since it had a more pronounced effect on plaque load than F19D/L34P. Both glial fibrillary protein (GFAP)-positive astrocytes and Iba1-positive microglia prominently outline amyloid plaques in the cortex of untreated APP/PS1 mice (FIGS. 5A, 5B). Indeed, the surface area of both GFAP and Iba1 exceeds that of Aβ immunostaining in untreated transgenic mice (FIG. 5C). Lifelong treatment with virally-delivered Aβ F20P significantly diminished staining for both of these markers, suggesting that, in some embodiments, neuroinflammation decreases with amyloid load.

An increase of cortical GFAP staining was also observed in virally-injected non-transgenic mice compared with uninjected controls (FIGS. 5A, 5C). The GFAP-positive astrocytes were largely restricted to a band neighboring the corpus callosum, and the effect was specific for astrocytes: Iba1 levels in non-transgenic mice were unchanged by viral exposure (FIGS. 5B, 5C). The same pattern of peri-callosal GFAP staining can be discerned in F20P-treated APP/PS1 mice, however, this area also contained plaque-associated astrocytosis that confounded quantitation (FIG. 5A).

Co-immunostaining for human Aβ (6E10) and GFAP was conducted in 7.5 mo old NTG mice. Although a few astrocytes were detected which expressed human Aβ, these co-labeled cells comprised only a small fraction of the GFAP+ population. These data suggest that, in some embodiments, while viral expression of variant Aβ peptide may cause mild astrogliosis on its own, the net effect in a model of amyloidosis is to abate the severity of chronic neuroinflammation commensurate with the reduction in plaque load.

### Example 2—Deciphering the Biophysical Mechanism of Interactions Between Variant and Wild-Type Aβ Peptides

Forms adopted by variant peptides in solution, both in isolation and in the presence of wild-type Aβ, are determined to ensure that their characteristics are appropriate for use in vivo.

A. Analysis of Structures Adopted by Aβ Peptides Upon Prolonged In Vitro Incubation

Structures formed by each variant peptide are assessed using four main assays: circular dichroism (CD) spectroscopy, size-exclusion chromatography (SEC), All/OC dot blot assay, and transmission electron microscopy (TEM). Initial experiments determine the secondary and tertiary structures formed by the isolated F20P and F19D/L34P peptides, how these change over time under varying incubation conditions, and whether either assembles into quaternary structures indicative of aggregation. Secondary structures of each peptide are monitored by far-UV CD spectroscopy and tertiary/quaternary structures by near-UV spectroscopy, using spectral and thermal scans with varying protein concentration and solvent conditions. Kinetic CD spectroscopy measurements will complement equilibrium assays to evaluate the temporal evolution of peptide folding and unfolding. Using data gleaned from CD spectroscopy to guide the timing of lag, exponential, and plateau phases for each peptide, samples are removed for SEC to determine whether monomeric peptide converts to small oligomers and larger structures. Separate timed samples are removed for All/OC dot blot to test for the formation of non-fibrillar and pre-fibrillar oligomeric states. A final set of samples is collected from plateau-stage reactions (>24 hr) for negative stain TEM imaging to check for 1) the presence or absence of structured fibrils and their periodicity and width where appropriate, 2) the appearance of smaller regular structures suggestive of oligomers or protofibrils, or 3) irregular structures suggestive of amorphous aggregation as previously observed in proline substitution variants of amyloidogenic peptides.

B. Analysis of Structures Created Upon Co-Incubation of Wild-Type Aβ Monomer with Aβ Variants

CD spectroscopy, SEC, All/OC dot blot, and TEM are used to assess the structures formed by co-incubation of F20P or F19D/L34P with wild-type Aβ.

C. Ratio of Variant Peptide Needed to Prevent the Aggregation of Wild-Type Aβ42 and Minimal Concentration Needed to Disassemble Pre-Formed Fibrils

In some embodiments, an equimolar mixture of variant Aβ peptide prevents aggregation of wild-type Aβ42, but a lower ratio of variant:wild-type suffices. While most peptide inhibitors require supra-stoichiometric concentrations to prevent wild-type Aβ aggregation, several candidates show efficacy at ratios as low as 1:50 inhibitor:wild-type. ThT assay is used to determine whether F20P or F19D/L34P can reduce or prevent the fibrillization of wild-type Aβ42 at substoichiometric concentrations down to 1:50 variant:wild-type. Parallel experiments can identify the minimum concentration of variant peptide needed for disassembly of pre-formed Aβ42 fibrils. In some embodiments, as little as 5 μM of F19D/L34P is sufficient to significantly lower ThT fluorescence of fibrils made from 10 μM WT Aβ starting material; in some embodiments, lower concentrations of F19D/L34P achieve the same effect and F20P may be equally efficient for fibril disassembly.

D. Analysis of Structures that Emerge from the Disassembly of Wild-Type Aβ Fibrils by Variant Aβ Peptides

In some embodiments, wild-type Aβ fibrils lose ThT fluorescence over time upon addition of F19D/L34P peptide, suggesting that, in some embodiments, this variant promotes fibril disassembly. Structures arising from the decomposition of wild-type Aβ fibrils are determined in the presence of variant peptides. Wild-type Aβ42 fibrils are produced and exposed to 10 μM F20P or F19D/L34P for analysis by CD spectroscopy, SEC, dot blot, and TEM as described herein. A parallel experiment is conducted using the lowest concentration of F20P or F19D/L34P capable of significantly lowering ThT fluorescence in the disassembly assay described above.

E. Effect of Co-Incubation of Variant Peptide with Wild-Type Aβ on Cytotoxicity

Structures formed by F20P and F19D/L34P variants may abate cytotoxicity of wild-type Aβ42 aggregates. In some embodiments, co-incubation of either variant with wild-type Aβ42 during oligomer formation reduces subsequent cell death in N2a cultures. Studies are conducted in cells lines and primary neuronal cultures, and the products of Aβ fibril disassembly by variant peptides are tested in both cell systems. Reaction products for Aβ42 assembly and disassembly are tested for each variant, at equimolar and minimal effective peptide ratios, from 0.1 and 10 μM, and cell viability measured by MTT assay.

Discussion

These experiments provide a biophysical understanding of how variant peptides prevent the aggregation of wild-type Aβ and how they promote the disassembly of pre-formed fibrils. By evaluating CD spectra under differing temperature, concentration, and solvent conditions, for both equilibrium and kinetic reactions, the physical structures that emerge from the interaction of wild-type and variant peptides can be discerned. In some embodiments, compared to wild-type Aβ, isolated variants remain in a predominantly random coil state, and co-incubation with wild-type decreases the emergence of structured oligomeric and fibril conformations. In some embodiments, SEC analysis confirms the predominance of monomeric peptide over oligomeric or fibril states both for isolated Aβ variants and upon co-incubation with wild-type Aβ. In some embodiments, TEM analysis of end-stage incubations detects fibrils only from wild-type Aβ42 and not from either variant alone or upon co-incubation with wild-type Aβ42.

Alternatively, in some embodiments, SEC analyses reveals the appearance of oligomeric species alongside a reduction in monomer, but without a substantial rise in larger species, accompanied by a shift in the CD spectra indicating a greater proportion of R-sheet structure over time, but not to the extent observed upon fibril formation. In some embodiments, annular or small protofibril structures are seen in TEM, which may also react with A11/OC. In some embodiments, such intermediates also occur following disassembly of pre-formed fibrils. In some embodiments, these intermediates are less toxic than those formed by wild-type Aβ.

### Example 3—Determination of how Dosage, Timing, and Route of Variant Aβ Administration Influence Efficacy In Vivo

A. Analysis of Lifelong Variant Aβ Expression in APP Mice

In some embodiments, lifelong expression of variant Aβ peptides significantly influences plaque development in APP/PS1 mice. A group of APP/PS1 mice treated with AAV encoding scramble Aβ peptide is added. This extra control ensures that the effect seen from treatment with variant Aβ is due to the peptide and not to a side effect of viral transduction. Determination of whether expression of variant Aβ affects APP expression or processing in vivo is also determined. In some embodiments, the amyloid-lowering mechanism of variant Aβ is due to its binding of wild-type Aβ after release from the cell, and full-length APP, sAPP, and CTF by Western blot are measured to ensure this is the case. Finally, in some embodiments, a second APP model, the APP^{NL-F} knock-in mouse line, confirms findings from APP/PS1 mice. Because KI mice develop plaques later than APP/PS1 mice, the KI animals provide an opportunity to test whether prolonged expression of variant Aβ at levels higher than endogenous peptide causes any unforeseen effects on pathology or neuronal survival. Both APP/PS1 and APP^{NL-F} KI mice are injected icv with variant or scramble AAV at P0 and harvested for analysis at 7.5 or 15 mo respectively. Outcome measures for this and subsequent experiments are histological plaque burden (Aβ and Thioflavin-S), human Aβ ELISA, and human oligomeric Aβ ELISA (IBL, 82E11).

B. Ratio (Dosage) of Variant Aβ Relative to Wild-Type Aβ Needed for Effect

In some embodiments, equimolar mixture of variant and wild-type Aβ is sufficient to arrest aggregation of wild-type peptide (FIG. 1B). Thus, in some embodiments, the effective dose for variant Aβ in vivo is considerably lower. The dose-response relationship between the production of variant Aβ and the accumulation of total Aβ in vivo is tested. Viral expression in vivo is mosaic in nature with not all cells transduced, but because the peptide is secreted, only a fraction of cells need to be transduced in order to expose a broad area to variant peptide. In some embodiments, the density of viral expression in the mouse brain is readily adjusted by controlling the titer of injected virus. Here, whether there is a threshold level of variant Aβ needed to slow plaque formation in vivo is determined. ELISA is used to empirically measure the ratio of F20P variant:wild-type Aβ, using the level of variant Aβ at each titer in non-transgenic mice for comparison with the level of wild-type human Aβ measured in young untreated APP/PS1 mice. While the dose-response relationship determined from this experiment applies to the APP/PS1 model, in some embodiments, it also provides valuable information about the relative exposure of variant Aβ needed for effect. APP/PS1 and non-transgenic mice are icv injected with F20P AAV at P0 and harvested for analysis 7.5 mo later. Viral dilutions are informed by previous vitro studies and range from ˜0.05 to 1× of previously used titers.

C. Ventricular Administration Phenocopies the Effect of Parenchymal Delivery

In some embodiments, variant Aβ need not be expressed by neurons to inhibit aggregation of wild-type Aβ in the extracellular space. In some embodiments, expression in any cell type with access to the interstitial fluid is sufficient to deliver variant Aβ widely across the brain. In some embodiments, the ependymal lining of the lateral ventricles is ideal for delivering secreted proteins into the brain via CSF. Sufficiency of delivery of variant Aβ into the CSF to reduce Aβ aggregation across the cortex as observed with neuronal secretion is tested. APP/PS1 and non-transgenic animals are injected with AAV1 encoding F20P or scramble Aβ to transduce the choroid plexus. Injections are done between P3 and P7 after the ependymal-brain barrier matures. Non-transgenic animals are used to measure the concentration of variant Aβ reaching the cortex following ependymal transduction for comparison with that attained by neuronal transduction. APP/PS1 mice are harvested at 7.5 mo for analysis.

D. Variant Aβ maintains benefit if introduced after plaque onset

In vitro studies indicate that, in some embodiments, variant Aβ promotes disaggregation if introduced after fibril formation. The potential for variant Aβ to clear pre-existing aggregates from the brain is examined. This experiment takes advantage of AAV serotype PHP.eB to broadly transduce cells in the adult brain. APP/PS1 and non-transgenic siblings are retro-orbitally injected at 7.5 mo of age with PHP.eB virus carrying F20P variant Aβ or scramble peptide. The human synapsin promoter is used for neuron-specific expression; viral titer for injection is empirically matched for each preparation within the published range of 1×10¹¹ to 1×10¹² gc/mouse. Non-transgenic animals are used to measure the concentration of variant Aβ in cortex following adult injection for comparison with that attained by treatment at P0. Animals are harvested for analysis 3 and 6 mo after treatment and compared to mice harvested without treatment at 7.5 mo of age.

Discussion

These experiments test how the dosage, timing, and route of administration influence the efficacy of variant Aβ in vivo. In some embodiments, expression of F20P and to a lesser extent F19D/L34P diminish plaque burden and Aβ load in the expanded cohort of APP/PS1 mice compared to both uninjected APP/PS1 mice and to the newly added control group expressing scramble Aβ peptide. In some embodiments, the same effect is observed in APP^{NL-F}, however, their later onset means they will overexpress variant Aβ for a longer time before harvest and will produce more variant relative to non-transgenic than APP/PS1 mice.

In some embodiments, there is a minimal ratio of variant-to-wild-type Aβ needed to slow plaque formation in vivo and that this is roughly parallel to the minimum ratio needed for effect in vitro. Above this ratio, in some embodiments, a graded degree of plaque reduction albeit with a less defined dose-response relationship than in vitro is observed. Non-transgenic animals are used to compare the concentration of variant Aβ reaching the cortex via CSF to that attained by neuronal expression to determine if CSF delivery attains cortical levels of variant Aβ equal or greater than the minimum effective dose for neuronal expression. If they do not, alternative serotypes for ventricular expression (i.e., AAV4) or alternative promoters active in the choroid plexus (i.e., Prlr, Spint2, F5) are used to improve delivery.

These experiments are advanced into cognitive testing, synaptic analysis, and electrophysiology in follow-up studies once a basic understanding of the Aβ species is established from exposure to variant peptide, identification of the optimal treatment regimen to allay or reverse Aβ accumulation in vivo, and determination of whether unintended neuroinflammation may confound physiological measures.

### Example 4—Interrogation of the Neuroimmune Reaction to Variant Aβ as a Possible Accomplice to Plaque Reduction

In some embodiments, neonatal viral transduction induces a minor and cell-specific neuroimmune response in the brain, but analyses have been limited to morphological markers and reveal nothing of underlying molecular changes. RNA profiling is used to assess a wide swath of neuroimmune markers for alterations due to viral expression alone or in the context of amyloid pathology. Evolution of the neuroimmune profile with age is tested to determine whether transduction of the adult brain elicits a more marked response than initially observed in mice injected as neonates. Histological analyses of astrocytes and microglia are completed to determine how plaque load, viral exposure, and cellular response interact following variant Aβ treatment following intervention at different stages of disease. In some embodiments, effects are due to astrocytic reactivity, while in other embodiments, effects are attributable to the biophysical effect of variant Aβ on wild-type peptide. In some embodiments, the biophysical effect trumps the astrocytic response. Empirical testing is performed to establish the utility of gene therapy as a platform for self-inhibition in AD other protein aggregation diseases.

A. Upregulation of Transcriptional Markers of Neuroinflammation in Response to Viral Delivery of Variant Aβ and Effect of Age of Viral Delivery on Response

In this experiment, whether the introduction of AAV expressing a non-native protein elicits a neuroinflammatory response in the brain is addressed. The focus is initially on non-transgenic animals to avoid the confounding effect of plaque-associated gliosis in APP/PS1 animals. Animals for this and subsequent experiments are generated as in Example 3: a small portion of frontal cortex (˜20-40 mg) is collected from one hemisphere of each animal for total RNA extraction, leaving the remaining cortex for ELISA and Western studies of Example 3. The contralateral hemisphere is fixed for histological studies. Here, the transcriptional profiles of non-transgenic control mice are compared to siblings treated with AAV-F20P or scramble peptide at P0 or 7.5 mo. Non-transgenic animals injected at P0 and harvested at 15 mo are also included to assess the impact of prolonged viral expression on the neuroinflammatory response. Transcription is profiled using the Nanostring nCounter Neuroinflammation Panel for mice which covers 756 genes spanning a broad range of potential neuroimmune responses. Unlike RNA sequencing, this platform yields direct digital measurement of mRNA molecules for linear quantitation without template amplification. Expression data are analyzed using nSolver software to compare gene expression in animals treated with F20P or scramble peptide to uninjected controls for each combination of treatment/harvest ages. Second tier analyses are obtained from the Baylor College of Medicine Multi-Omics Data Analysis Core for pathway and gene set enrichment. RNA sequencing is also available and provides a broader readout of potential changes.

B. Effect of Virally-Delivered Aβ Variants on the Neuroinflammatory Response

Addressed is whether the introduction of AAV and its non-native peptide cargo alter the neuroinflammatory response to amyloid deposits. Cortical transcriptional profiles from APP/PS1 and APP^{NL-F} mice injected with F20P or scramble Aβ at P0 or 7.5 mo are compared to uninjected APP/PS1 and APP^{NL-F} siblings from each experiment. The Nanostring nCounter Neuroinflammation Panel is used for expression profiling of the neuroimmune response in APP models.

C. Differing Morphology and Distribution of Neuroinflammatory Cells Upon Viral Expression of Variant Aβ

The cellular profile of the neuroimmune response to viral delivery of variant Aβ is examined. Immunofluorescence is used to detect microglia (Iba1 as a pan-microglial marker, P2RY12 for homeostatic microglia, CD68 for phagocytic microglia, and MHCII for inflammatory microglia) and astroctyes (Aldh111 as a pan-astrocyte marker, and GFAP preferential for reactive astrocytes) in non-transgenic, APP/PS1, and APP^{NL-F} mice. Mice are harvested, following injection with F20P or scramble peptide at P0 or 7.5 mo for comparison with uninjected mice. Outcome measures include the qualitative morphology and the quantitative cell number and spatial distribution of cortical neuroimmune cells per unit area in non-transgenic animals, or in relation to amyloid plaques in APP models.

Discussion

This work surveys the neuroinflammatory response to viral delivery of non-native proteins by transcriptional and cellular markers. These experiments also examine whether the amyloid reduction observed in pilot studies may be aided by a neuroinflammatory response to treatment. In some embodiments, mild astrocytosis may accompany viral delivery in non-transgenic mice, however, both astrocytosis and microgliosis are reduced commensurate with plaque load in F20P-treated APP/PS1 animals. In some embodiments, these molecular and histological experiments allow resolution of this discrepancy and support a mechanism based on Aβ steric inhibition rather than neuroinflammatory phagocytosis.

In some embodiments, if mRNA or histological profiles suggest elevated inflammation in animals treated with variant Aβ, profiles in mice treated via neuronal transduction are compared to those treated via CSF. CSF delivery limits viral exposure but not the spread of variant Aβ, and comparison to neuronal expression should pinpoint whether virus or peptide is to blame. Complementary studies in vitro directly test the microglial/astrocytic response to variant Aβ peptide using purified primary cell cultures and recombinant protein. Treatment efficacy in the presence and absence of astrocytes/microglia is also compared to rule out a role for neuroinflammation in plaque reduction.

Finally, in some embodiments, the mRNA panel indicates that additional cell types such as circulating macrophages, T- or B-cells are involved in any neuroinflammatory response, and FACS sorting of brain tissue is used to identify the cellular components and extent of neuroinvasion. Either the delivery route (i.e., ventricular rather than neuronal) or the capsid (AAV9, used clinically vs. AAV8/PHP.eB, used experimentally) is modified for future studies to abate any treatment-related neuroinflammation.

In some embodiments, if either the mRNA marker panel or histological staining confirm an elevated response in virally-treated animals, additional experiments are undertaken to map the time course of neuroinflammatory changes following viral delivery at either P0 or in the adult, as needed.

### Example 5—Variant Aβ Slows AD Aggregate Seeding

Tested is whether gene therapy with variant Aβ can diminish the potential for pathogenic protein seeds to accelerate disease progression in vivo. These experiments provide the opportunity to test whether variant Aβ is limited to preventing seed formation from monomer or whether it can also suppress growth of existing seeds. Moreover, these experiments test pathogenic seeds from human AD tissue, which represent a more diverse and realistic challenge than genetic models alone. First experiments examine whether preventative expression of variant Aβ can slow plaque formation caused by inoculation with human AD extract rich in Aβ seeds. Final experiments test the potential for Aβ reduction to dampen the secondary seeding of tau in amyloid-bearing mice.

A. Mechanism of Variant Aβ Effect on the Rate and Extent of Amyloid Seeding in Pre-Deposit Mice

Tested is whether lifelong expression of variant Aβ can abate the accelerated amyloidosis caused by inoculation with exogenous AD brain extract. Aβ extract for in vivo seeding is prepared from two pathologically confirmed AD subjects and from one age-matched healthy control. Frozen frontal cortex samples are obtained. Protein extracts for injection are homogenized in PBS, sonicated, and clarified. Extract is characterized by A11/OC dot blot for oligomeric Aβ, ELISA for total Aβ concentration, and protein misfolding cyclic amplification (PMCA) assay for seeding capacity. At birth, APP/PS1 mice are administered AAV encoding F20P variant or scramble Aβ, followed 1 month later by bilateral injection of AD or healthy control brain extract into the hippocampus and overlying cortex. Animals are harvested 3-5 months later, prior to the normal onset of plaques in this model, for Aβ immunohistochemistry and ELISA to assess the severity and spread of Aβ aggregation in animals expressing F20P vs scramble.

B. Variant Aβ Affects Seeded Tau Pathology in Amyloid-Bearing Mice

Final experiments test whether the presence of variant Aβ can prevent or slow the formation of tau neuropathology in amyloid-bearing mice. Tau extracts for injection are prepared from cortical grey matter by differential centrifugation of sarkosyl-insoluble brain homogenate as originally described by the Lee group. Extract is characterized by PHF1 Western blot and ELISA for total tau concentration, and tested for seeding potential using tau RD FRET biosensor cells (available from ATCC). Once AD-tau extract performs as expected in vitro, in vivo experiments are conducted by administering AAV encoding F20P or scramble Aβ to neonatal APP/PS1 mice. Twelve months later, to ensure sufficient amyloid load, AD-tau extract is bilaterally injected into the hippocampus and overlying cortex. Animals are harvested 3 or 6 months later for tau immunohistochemistry (AT8, AT180, MC1) and amyloid immunostaining and histology (Thioflavin-S) to measure the spread and total area of phospho-tau immunostaining relative to amyloid load in animals expressing F20P vs scramble.

Discussion

In some embodiments, lifelong expression of variant Aβ reduces amyloid formation in APP/PS1 mice, and diminished seeding of plaques and phospho-tau neurites by AD extracts is likely. In some embodiments, variant Aβ limits extension of injected AD-Aβ seeds as it does for seeds generated de novo. Similarly, in some embodiments, the emergence of phospho-tau neurites decreases as variant Aβ dampens formation of amyloid plaques needed to promote the secondary seeding of tau. Alternatively, in some embodiments, variant Aβ is unable to attenuate seeding by exogenous aggregates, suggesting that, in some embodiments, variant Aβ is sufficient to slow seed formation in APP mice, but either unable to prevent seed growth once present or to interact with seeds present in AD extract; however, this is unlikely due to the predicted mechanism of steric hindrance during fibril extension which should occur regardless of the starting material.

These experiments provide a critical foundation for future studies testing how the timing of treatment relative to inoculation governs efficacy. Tests are conducted to determine whether variant Aβ can thwart seeding if provided after plaque onset. Beyond timing, also examined is how dosage and route of administration influence progression from amyloid to tau.

### Example 6—Exemplary Methods

Preparation of WT Aβ42 or variant peptide stocks. Synthetic Aβ42 WT or variant peptides were purchased from Biomatik (Wilmington, DE). To prepare stock solutions of aggregate-free Aβ42 WT or variant peptides, powdered peptide was dissolved in 50% acetonitrile, frozen, and lyophilized overnight to remove any residual trifloroacetic acid. Lyophilized Aβ42 peptides were dissolved in HFIP (#105228, Sigma-Aldrich, St. Louis, MO). The Aβ-HFIP solution was incubated at room temperature (RT) for 30 min and then divided into aliquots. HFIP was evaporated overnight in a fume hood and then transferred to SpeedVac for 1 hr to remove any remaining traces of HFIP. Tubes containing the peptide film were kept over desiccant at −20° C. until used. Immediately prior to experimental use, lyophilized peptides were dissolved in DMSO to a final concentration of 5 mM and sonicated for 10 min in a bath sonicator.

Preparation of oligomeric and fibrillar WT Aβ. Oligomeric Aβ was generated by dissolving peptide in Ham's F-12 media (#30611040-1, Fisher Scientific, Pittsburgh, PA) to a final concentration of 100 um WT Aβ42 peptide or 100 μM WT+100 μM variant and then incubating at 4° C. for 24 hr without shaking. Fibrillar Aβ was generated by dissolving WT peptide in PBS to a final concentration of 100 um and incubating at 37° C. for 24 hr without shaking.

ThT assay to test kinetics of Aβ self-aggregation, competition with WT peptide, and fibril disassembly. Self-aggregation of Aβ42 WT or variant monomers was tested at a starting concentration of 10 μM in PBS containing 5 μM Thioflavin T (ThT). ThT fluorescence was measured using an Infinite M1000 Pro Plate Reader (Tecan, Mannedorf, Switzerland) at an excitation wavelength of 440 nm and emission wavelength of 485 nm. Reactions were incubated without shaking at 370 C then shaken for 5 sec prior to reading fluorescence. Competition between variant and WT Aβ was performed similarly, but using a starting concentration of 10 μM for each peptide in the mixture (1:1, WT:variant), with the exception of WT alone, which was tested at a final concentration of 10 μM. Fibril disassembly was assessed by mixing 10 μl of WT Aβ42 fibrils (described above) in a 1:1 ratio with 5, 10, or 20 μM monomeric F19D/L34P or F20P peptides in PBS containing 5 μM ThT. Fluorescence was measured without shaking every 24 hr for 48 hr of incubation at 37° C.

Oligomeric Aβ toxicity assay. N2a cells were grown in Eagle's minimal essential medium (EMEM) (#112-018-101, VWR, Radnor, PA) supplemented with 1×10⁴ U/ml penicillin/streptomycin (#15140-122, Life Technologies, Carlsbad, CA) and 10% fetal bovine serum (#MT35010CV, Fisher) at 37° C. in 5% CO₂. Confluent cells were trypsinized, diluted in EMEM containing 1% N₂ supplement to minimize cell growth, and then plated 5,000 cells/well in transparent flat-bottom 96-well plates (#07-200-89, Fisher). 10 μl of each oligomer preparation (WT Aβ alone or WT+variant, described above) or 10 μl of a 1:10 dilution was added to 90 μl of culture medium. Cell viability was determined 24 hours after treatment using a MTS assay (#G3582, Promega, Madison, WI), according to the manufacturer's directions. Briefly, assays were performed by adding 20 μl of AQueous One Solution Reagent directly to culture wells, incubating for 2 hr at 37° C. in 5% CO₂ atmosphere and then recording absorbance at 490 nm using an Epoch 2 spectrophotometer (Biotek, Winooski, VT).

Plasmid constructs. PCR was used to add the Gaussia luciferase signal peptide (GLSP, amino acids MGVKVLFALICIAVAEA. corresponding to SEQ ID NO:20) onto the N terminus of Aβ-CTF. First, a synthetic DNA for GLSP with partial Aβ sequence was made using oligo GLSP-1 5′ ATGGGCGTGAAGGTCCTGTTCGCCCTGATTTGCATCGCCGTCGCAGAGGCAGATG CAGA 3′ (SEQ ID NO:40) and oligo GLSP-2 5′ TCTGCATCTGCCTCTGCGACGGCGATGCAAATCAGGGCGAACAGGACCTTCACG CCCAT 3′ (SEQ ID NO:41). These oligonucleotides were annealed by incubation in a thermocycler programmed to start at 95° C. for 2 minutes and then gradually cool to 25° C. Second, Aβ-CTF carrying a partial GLSP sequence was generated by amplifying a plasmid containing the human APP wild-type sequence (pBS-hAPPwt-IRES-GFP) with forward primer 5′ GTCGCAGAGGCAGATGCAGAATTCCGACATGAC 3′ (SEQ ID NO:42) and reverse primer 5′ GCGCGGATATCCTAGTTCTGCATCTGCTCAAAG 3′ (SEQ ID NO:43). GLSP-Aβ-CTF was then generated by Gibson assembly from the two templates, joining the GLSP+partial Aβ to the Aβ-CTF+partial GLSP, using a forward primer to add a Kozak sequence 5′ GCGCGAAGCTTGCCACCATGGGCGTGAAGGTCCTGTT 3′ (SEQ ID NO:44) and reverse primer 5′ GCGCGGATATCCTAGTTCTGCATCTGCTCAAAG 3′ (SEQ ID NO:45). The resulting GLSP-Aβ-CTF fragment was digested with HindIII and EcoRV and subcloned into pAAV containing the CAG promoter and WPRE to create pAAV-GLSP-Aβ-CTF.

The GLSP-Aβ-CTF deletion series was constructed by cloning various GLSP-Aβ-CTF deletions into pAAV. The GLSP-Aβ-CTF deletion series was amplified by PCR from the full-length pAAV-GLSP-Aβ-CTF using a common forward primer with a series of reverse primers, and then digested with HindIII and EcoRV to ligate into pAAV.

Forward Primer:

Aβ-GLSP-F(KOZAK):
(SEQ ID NO: 46)
5′ GCGCGAAGCTTGCCACCATGGGCGTGAAGGTCCTGTT 3′.
Reverse primers:
Aβ-R(KK):
(SEQ ID NO: 47)
5′ GCGCGgatatcTTACTACTTCTTCAGCATCACCAAGGTG 3′;
Aβ-R(ML):
(SEQ ID NO: 48)
5′ GCGCGgatatcTTACTACAGCATCACCAAGGTGATGA 3′;
Aβ-R(LV):
(SEQ ID NO: 49)
5′ GCGCGgatatcTTACTACACCAAGGTGATGACGATCA 3′;
Aβ-R(IT):
(SEQ ID NO: 50)
5′ GCGCGgatatcTTACTAGGTGATGACGATCACTGTCG 3′;
Aβ-R(IV):
(SEQ ID NO: 51)
5′ GCGCGgatatcTTACTAGACGATCACTGTCGCTATGA 3′;
Aβ-R(IA):
(SEQ ID NO: 52)
5′ GCGCGgatatcTTACTACGCTATGACAACACCGCCCA 3′;

To build pAAV-GLSP-Aβ(F19D/L34P)-KK containing the F19D/L34P substitution, two PCR reactions were performed. A fragment of GLSP-Aβ(F19D/L34P)-KK was amplified from pAAV-GLSP-Aβ-KK using forward primer 5′ GCGCGaagcttGCCACCATGGGCGTGAAGGTCCTGTT 3′ (SEQ ID NO:53) and reverse primer 5′ ACCATGGGTCCAATGATTGCACCTTTGTTTGAACCCACATCTTCTGCAAAGTCCA CCAA 3′ (SEQ ID NO:54). A second fragment of GLSP-Aβ(F19D/L34P)-KK was amplified using forward primer 5′ TTGGTGGACTTTGCAGAAGATGTGGGTTCAAACAAAGGTGCAATCATTGGACCC ATGGT 3′ (SEQ ID NO:55) and reverse primer 5′ GCGCGGATATCTTACTACTTCTTCAGCATCACCAAGGTG 3′ (SEQ ID NO:56). The resulting fragments were joined by Gibson assembly using forward primer 5′ GCGCGAAGCTTGCCACCATGGGCGTGAAGGTCCTGTT 3′ (SEQ ID NO:57) and reverse primer 5′ GCGCGGATATCTTACTACTTCTTCAGCATCACCAAGGTG 3′ (SEQ ID NO:58). The resulting insert was digested with HindIlI and EcoRV and subcloned into pAAV to create pAAV-GLSP-Aβ(F19D/L34P)-KK.

For pAAV-GLSP-Aβ(F20P)-KK containing the F20P substitution, two PCR reactions were performed. A fragment of GLSP-Aβ(F20P)-KK was amplified from pAAV-GLSP-Aβ-KK using forward primer 5′ GCGCGAAGCTTGCCACCATGGGCGTGAAGGTCCTGTT 3′ (SEQ ID NO:59) and reverse primer 5′ TCTTCTGCAGGGAACACCAATTTTTG 3′ (SEQ ID NO:60). A second fragment of GLSP-Aβ(F20P)-KK was amplified using forward primer 5′ CAAAAATTGGTGTTCCCTGCAGAAGA 3′ (SEQ ID NO:61) and reverse primer 5′ GCGCGGATATCTTACTACTTCTTCAGCATCACCAAGGTG 3′ (SEQ ID NO:62). The resulting fragments were joined by Gibson assembly using forward primer 5′ GCGCGAAGCTTGCCACCATGGGCGTGAAGGTCCTGTT 3′ (SEQ ID NO:63) and reverse primer 5′ GCGCGGATATCTTACTACTTCTTCAGCATCACCAAGGTG 3′ (SEQ ID NO:64). The resulting insert was digested with HindIII and EcoRV and subcloned into pAAV to create pAAV-GLSP-Aβ(F20P)-KK. All restriction enzymes were purchased from New England Biolabs (Ipswich, MA, USA).

Collection of Aβ peptides from N2a conditioned media. N2a cells were grown in 6 well plates using Dulbecco's modified Eagle's medium (DMEM) (#12-604F, VWR) supplemented with 1×104 U/ml penicillin/streptomycin and 10% fetal bovine serum until approximately 90% confluent. Cells were then transfected with 2.5 ug/well of sequentially deleted APP-CTF sequences (e.g., pAAV-GLSP-Aβwt-KK) using Lipofectamine LTX (#15338030, Fisher) or with 14 μg/dish (10 cm) of pAAV-GLSP-Aβ(F20P)-KK. Twenty-four hours later, cells were washed with 1×Dulbecco's PBS, and a serum-reduced medium consisting of DMEM and 0.2% fetal bovine serum was added before the cells were returned to 5% CO₂ at 37° C. Conditioned media was harvested 24 hr later and centrifuged at 10,000 rpm for 5 min at 4° C. (2 ml/well×3 wells per IP reaction). To test the dependence of Aβ release on γ-secretase activity, cells were transfected as above, with the subsequent media replacement including 0.1 or 1 nM of LY411575 (#SML0506, Sigma). Supernatants were collected and supplemented with protease inhibitors (#5892970001, Sigma) and 0.01% NaN₃ (#S2002, Sigma) to prevent proteolytic degradation.

Aβ IP from conditioned media for immunoblot. Protein G Dynabeads (50 μl, #10003D, Fisher) were loaded with mouse anti-Aβ antibody 6E10 (1 ug, #SIG-39320, BioLegend, San Diego, CA) for 1 hr at RT. Conditioned media from transfected N2a cells (6 ml) was incubated with 50 μl of Dynabead-6E10 complex in a rotator overnight at 4° C. The following day, beads were collected using by magnetic separation to remove the supernatants. Beads were washed 3× with PBS containing 0.02% Tween-20. Immunoprecipitated complexes were eluted with 50 mM glycine pH 2.8. Samples were then denatured with an equal volume of 2× Laemmli sample buffer for 10 min at 70° C. and electrophoresed on 16.5% Criterion Tris-Tricine gels (#3450063, Bio-Rad, Hercules, CA). Proteins were transferred to nitrocellulose using a Trans-Blot Turbo Transfer System (#170-4159, Bio-Rad). Membranes were blocked in PBS containing 0.1% Tween-20 and 5% non-fat dry milk for 1 hr at RT, and probed overnight at 4° C. with 6E10 diluted 1:5000 in blocking solution. Antibody binding was detected using mouse anti-IgG secondary antibody conjugated with IRDye, diluted 1:20,000 in block (#26-32210, LI-COR, Lincoln, NE). Blots were imaged with an Odyssey Fc Imager and analyzed with Image Studio software (LI-COR).

Aβ IP from conditioned media for MS. Approximately 60 mL of N2a conditioned media containing Aβ F20P was incubated with 10 μg mouse anti-Aβ antibody 6E10 (#SIG-39320; BioLegend, San Diego, CA, USA), divided into two batches of 30 mL each, on a rotator overnight at 4° C. The following day, Protein G Dynabeads (100 μL; #10003D; Fisher Scientific) were added to the media and incubated for 2 h at 4° C. Beads were collected with magnetic separation to remove the supernatant, then washed 3 times with PBS containing 0.02% Tween-20, followed by two washes in PBS without detergent. Finally, immunoprecipitated complexes were eluted in 80 μL of 10% formic acid solution for MS analysis.

MS. The eluted immunoprecipitates from brain homogenate and culture media expressing Aβ F20P were dried with a SpeedVac and resuspended in 50 μL of 20% formic acid. 1 μL of IP reaction was loaded onto a 10-cm, 100-μm inner-diameter C3 column (ZORBAX 300SB-C3; 300 Å 5 μm), self-packed into fused silica, pulled to form a nanoelectrospray emitter. Online high-performance LC (HPLC) was performed on a Thermo Scientific U3000 RSLCnano ProFlow system. A 50-min linear gradient from 0% buffer B to 35% buffer B, using buffer A: 2% acetonitrile, 0.1% formic acid, and buffer B: 98% acetonitrile and 0.1% formic acid. The column eluant was introduced into a Thermo Scientific Orbitrap Fusion Lumos by nanoelectrospray ionization. A static spray voltage of 2,200 V and an ion transfer tube temperature of 320° C. were set for the source.

MS1 was performed by the Orbitrap at a 60-k resolution setting, in positive mode with quadrupole isolation. An automatic gain control (AGC) target of 5.0e6 with 200 ms maximum injection time, two microscans, and a scan range of 350-2,000 mass-to-charge ratio (m/z) were used. Target precursor m/z selected for MS2 fragmentation included monoisotopic and most abundant masses for +4, +5, and +6 Aβ F20P 38, 40, and 42 amino acid unmodified ions. Higher-energy collisional dissociation (HCD) fragmentation with a normalized collision energy of 45% was used. MS2 acquisition was performed using the Orbitrap with the 15-k resolution setting, an AGC target of 1e6, a max injection time of 100 ms, a scan range of 150-2,000 m/z, and three microscans. Next, the masses of α-secretase-cleaved Aβ 1-16 were specifically targeted along with an uncleaved and partially cleaved precursor (with/without signal peptide and residual TM domain) for MS analysis to enhance sensitivity for these targets. The parameters were duplicated with a targeted mass list containing these alternatively cleaved peptides.

Data processing was performed by a custom analysis suite, as described previously. The Aβ F20P 38, 40, and 42 peptides, α-secretase-cleaved Aβ 1-16, along with an uncleaved and partially cleaved precursor peptide (with/without signal peptide and residual TM domain) were used as template sequences for the search. Representative spectra for unmodified Aβ F20P are shown.

Viral packaging. All AAVs were prepared by the Gene Vector Core at Baylor College of Medicine using a method similar to one previously described. HEK293T subclone 1F11 cells were grown in DMEM (#CM002-050, GenDEPOT Corp., Barker, TX) supplemented with 10% FBS (#97068-085, VWR) and 1× antibiotic/antimycotic (#CA002-010, GenDEPOT). Serotype 8 AAV was prepared by co-transfection of three plasmids (expression vector (1.14 ug/15-cm plate), p5E18-VD2/8 Rep-Cap plasmid (4.57 ug/plate), and pAdDF6 helper plasmid (2.29 ug/plate) using 24 μl/plate of iMFectin Poly DNA Transfection Reagent (#I7200-101, GenDEPOT). AAV purification was performed using a protocol based on Ayuso et al. but with 15% iodixanol containing 0.75 M NaCl. Three days after transfection, cells were collected while the media was retained for subsequent polyethylene glycol (PEG) precipitation. The cell pellet was re-suspended in 1 ml per plate of 50 mM Tris pH 8.0 containing 5 mM MgCl₂ and 0.15 M NaCl, lysed by adding 0.1 volume of 5% sodium deoxycholate for 30 min at RT, and then incubated with 10 μg/ml of DNase I and RNase A for 1 hr at 37° C. Cell lysates were clarified by centrifugation at 5,000×g for 10 min at 4° C. The culture media was incubated with 10 μg/ml of DNase I and RNase A for 1 hr at 37° C. and then incubated overnight at 4° C. in 8% PEG (stock 40% PEG 8000 plus 2.5 M NaCl). AAV was collected from this mixture by centrifugation at 2,500×g for 30 min. The pellet containing AAV was resuspended in a minimal volume of HBS (50 mM HEPES, 0.15 M NaCl, 1% sarcosyl, and 20 mM EDTA pH 8.0). Cell-associated and secreted AAV preparations were combined for iodixanol density centrifugation. The resulting AAV particles were dialyzed against Mg- and Ca-free PBS using an Amicon Ultra-15 centrifugal filter (100,000 kDa nominal limit, Millipore, Burlington, MA,) and the titer determined by real-time PCR.

Mice. Wild-type ICR animals purchased from the Center for Comparative Medicine at Baylor College of Medicine were used generate offspring for P0 viral injection. Wild-type pups from these litters were virally injected at P0 as described below and harvested to assess neocortical viral expression at 1 mo of age. Male APPswe/PS1dE9 bigenic mice were obtained from the Mutant Mouse Resource and Research Center at Jackson Laboratory (stock #34832-JAX, B6.Cg-Tg(APPswe,PSEN1dE9) 85Dbo/Mmjax). These were mated with wild-type C57BL/6J mice to establish a backcross colony, or with FVB/NJ females to generate F1 offspring for study. Both male and female, non-transgenic (wild-type) and APP/PS1-positive offspring were used for P0 viral injection to evaluate variant Aβ expression at 7.5 months of age. All animal experiments were reviewed and approved by the Baylor College of Medicine Institutional Animal Care and Use Committee, and conform to relevant regulatory standards.

P0 intraventricular injections Stereotaxic injection of AAV into the lateral ventricles of neonatal mouse pups was performed as described previously. Within 6 hr after birth, neonates were collected from the cage and prepared for injection by cryoanesthesia. Following cessation of movement, viral solutions of 4×10⁶ TU/μl diluted in sterile PBS containing 0.05% trypan blue were injected into the lateral ventricles using a 10 μl syringe (Hamilton Company, Reno, NV, #7653-01) fitted with a 32 gauge needle (Hamilton, #7803-04, RN 6PK PT4). Two sites per hemisphere were injected with 1 μl of viral solution per site using a neonatal stereotaxic device (X, Y, Z) (±0.8, ±1.5, −1.5 mm) and (±1.35, ±2.0, −1.7 mm) from lambda. Injected pups were placed on a warming pad to regain normal color and movement before being returned to their biological mother for care.

Tissue harvest. Wild-type ICR mice were studied for viral spread and expression at 3 weeks of age; transgenic APP/PS1 animals and their non-transgenic siblings were studied for Aβ level, plaque load, and gliosis at 7.5 months of age. Mice were killed by sodium pentobarbital overdose and transcardially perfused with PBS and heparin. Brains were removed and dissected along the midline. The rostral half of the left cortex was snap-frozen on dry ice for biochemistry. The right hemisphere was immersion fixed in 4% paraformaldehyde for 48 hr at 4° C., cryoprotected in 30% sucrose at 4° C., and sectioned at 35 m for histology.

Tissue homogenization. Frozen frontal cortex was sonicated in PBS containing 5 mM EDTA, 1× protease inhibitor (#05892970001, Roche, Basel, Switzerland) and 1× PhosSTOP (#04906845001, Roche) and centrifuged at 100,000×g for 30 min at 4° C. The pellet was resuspended in an equal volume of PBS containing 1% Triton X-100 (PBS-X) and mixed by gentle rotation for 30 min at 4° C. Samples were centrifuged at 100,000×g for 30 min at 4° C., and the supernatant saved as the PBS-X soluble fraction. The pellet was resuspended in an equal volume of 5 M guanidine hydrochloride in 50 mM Tris pH 6.8 and mixed by gentle rotation overnight at RT. Samples were centrifuged at 16,000×g for 30 min at RT, and the supernatant saved as the guanidine soluble fraction.

Aβ IP from brain extract for MS. Brain extract from WT ICR mice injected with AAV-F20P at P0 and harvested at 3 weeks was used for IP-MS. 500 μL of PBS-X soluble extract was mixed with 500 μL radioimmunoprecipitation assay (RIPA) buffer (PBS containing 5 mM EDTA, 0.5% Igepal, 0.5% sodium deoxycholate, 0.2% SDS, and protease inhibitor) and then incubated with 5 μg mouse anti-Aβ antibody 6E10 overnight at 4° C. on a rotating platform. The following day, Protein G Dynabeads (50 μL) were added to the homogenate and incubated for 2 h at 4° C. Beads were collected with magnetic separation to remove the supernatant and then washed 3 times with PBS containing 0.02% Tween-20, followed by two washes in PBS without detergent, before the immunoprecipitated complexes were eluted with 40 μL of 10% formic acid solution for MS.

Meso Scale Discovery assay The concentration of human Aβx-40 and -42 in frontal cortex extracts prepared in PBS-X (3 weeks of age) or guanidine (7.5 months of age) was measured using Multiplex Aβ Peptide Panel 1 (#K15200E, Meso Scale Diagnostics, Rockville, MD). The assay was performed essentially as instructed by the manufacturer, using the provided 6E10 antibody for capture (human Aβ 1-16) and end-specific 40/42 antibodies for detection. Samples were diluted to stay within the linear range of the assay, requiring dilution of 1:1 for PBS-X and 1:250 for guanidine. Initial dilution of the PBS-X and guanidine fraction was done in PBS containing 1% protease-free BSA (#820451, MP Biomedical, Santa Ana, CA). The final working dilution was prepared with Diluent 35 included in the kit; Aβ blocking reagent was not used. Samples were read on a SECTOR Imager 6000 and concentrations calculated using Discovery Workbench software (Meso Scale Diagnostics).

Immunofluorescence A 1/12 series of sections was rinsed with TBS and blocked with TBS containing 0.1% Triton X-100 and 10% normal goat serum for 1 h at RT before overnight incubation at 4° C. with mouse anti-GFAP (#G3893, Sigma), rabbit anti-Iba1 (#019-19741, Waco, Richmond, VA), or mouse anti-Aβ antibody 6E10 (BioLegend) plus rabbit anti-Aβ antibody Y188 (#ab32136, Abcam, Cambridge, United Kingdom), each diluted 1:500 in blocking solution. After several washes in TBS, sections were incubated with Alexa Fluor-568 goat-anti rabbit and Alexa Fluor-488 goat-anti mouse secondary antibodies (#A-11036 and #A-21121, Life Technologies) diluted 1:500 in block for 2 h at RT. GFAP/Iba1 sections were washed with TBS before being counterstained for 8 min at RT with 0.002% thioflavin-S diluted in TBS. Thioflavine-stained sections were washed twice in 50% ethanol followed by several washes in TBS before being mounted. All sections were mounted onto Superfrost Plus slides (#12-550-15, Fisher) and coverslipped with Vectashield mounting medium (#H1400, Vector Laboratories, Burlingame, CA).

Immunohistochemistry. For labeling amyloid-beta plaques, free-floating sections were washed of cryoprotectant with TBS, incubated in 88% formic acid for 1 min, and then rinsed with TBS. Endogenous peroxidases were quenched with 0.9% H₂O₂ in TBS+0.1% Triton X-100 (TBS-T) for 30 min at RT. Following washes in TBS, sections were blocked with 5% normal goat serum in TBS-T for 1 hour at RT. Sections were incubated at 4° C. overnight with primary antibody (1:500, Rb anti-Aβ, ThermoFisher/Zymed, 71-5800) diluted in blocking buffer. The following day, sections were washed in TBS and incubated with secondary antibody for 2 hr at RT (1:500, biotinylated Gt anti-Rb, VECTASTAIN Elite ABC Kit, Vector Labs, PK-6101). Thirty min before use, A+B reagent was made using 50 μl of each solution in 5 ml of TBS. Sections were washed several times in TBS, then incubated in A+B reagent for 90 min at RT. Sections were again washed with TBS, and then developed with filtered DAB solution (Sigma, D4293) and quenched with TBS washes. Sections were mounted on SuperFrost Plus slides (Fisher, 12-550-15) and dried overnight. Slides were processed through an ethanol series (70%, 95%, 100%, xylene) before being coverslipped with Permount (Fisher, SP15-100).

For astrocyte and microglial labeling, sections were processed as above, with the following modifications. Endogenous peroxidases were quenched using 0.9% H₂O₂ in TBS+0.1% Triton-X100+0.05% Tween-20 (TBS-TT) for 30 min at RT. Blocking solution consisted of 5% NGS in TBS-TT. Primary antibodies were diluted in TBS-TT (1:1,000, Rb anti-GFAP, DAKO Z0334, or 1:1,000, Rb anti-Iba1, Wako 019-19741).

Quantification of Aβ, GFAP, and Iba1 surface area. Tiled images were acquired using a Zeiss Axio Scan.Z1 at 10× magnification (Carl Zeiss AG, Oberkochen, Germany). Exposure time and lamp intensity were constant for all sections. Two sagittal sections between 0.24 and 2.04 mm from bregma were randomly chosen for quantification. The frontal cortex anterior to the hippocampus was outlined as the ROI for analysis using Fiji 1.51W (NIH, USA). Images were converted to 8-bit and a threshold was applied using the Yen algorithm with the ROI as reference. The percent area above threshold with the ROI was used for quantitation.

Statistics. Statistical comparisons and graphing were done using GraphPad Prism 6.0 h. Comparisons of two groups were done by Student's t-test; comparisons of three or more groups were done by one or two-way ANOVA followed by Bonferroni post-test. Grubb's test was used to identify outlier data points (graphpad.com/quickcalcs/Grubbs1.cfm); this resulted in removal of 1 data point from FIG. 3E (F19D/L34P Aβ42); two from FIG. 3F (F19D/L34P Aβ40, and F20P Aβ42), one from FIG. 3G (7.5 mo F19D/L34P), and one from FIG. 4B (F20P). Graphs display group mean±SEM.

All of the methods disclosed and claimed herein can be made and executed without undue experimentation in light of the present disclosure. While the compositions and methods of this invention have been described in terms of certain embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

1-156. (canceled)

157. A method of treating or preventing a neurodegenerative disease, disorder, or condition in a subject, comprising administering to the subject a therapeutically effective amount of a composition comprising a vector encoding an amyloid beta (Aβ) peptide variant, wherein the Aβ peptide variant comprises an amino acid sequence having at least 80% identity with:
(SEQ ID NO: 3)
DAEFRHDSGYEVHHQKLVDFAEDVGSNKGAIIGPMVGGVVIA,
or a fragment or functional derivative thereof;
(SEQ ID NO: 4)
DAEFRHDSGYEVHHQKLVFPAEDVGSNKGAIIGLMVGGVVIA,
or a fragment or functional derivative thereof;
(SEQ ID NO: 5)
DAEFRHDSGYEVHHQKLVDFAEDVGSNKGAIIGLMVGGVVIA,
or a fragment or functional derivative thereof,
or
(SEQ ID NO: 6)
DAEFRHDSGYEVHHQKLVPFAEDVGSNKGAIIGLMVGGVVIA,
or a fragment or functional derivative thereof.

158. The method of claim 157, wherein protein misfolding, endogenous Aβ peptide aggregation, amyloid plaque formation, neuroinflammation, neurodegeneration, neuronal loss, or synaptic loss is prevented or decreased; tau levels, phosphorylation of tau, or phosphorylated tau levels are decreased; seeding of tau or seeding of endogenous Aβ peptide are slowed; and/or cognitive improvement is promoted.

159. The method of claim 157, wherein formation of endogenous Aβ peptide oligomers, protofibrils, fibrils, or plaques are prevented or decreased.

160. The method of claim 157, wherein cytotoxicity of endogenous Aβ peptide aggregate is prevented or decreased.

161. The method of claim 157, wherein the neurodegenerative disease, disorder, or condition is Alzheimer's disease, Parkinson's disease, Parkinson's disease dementia, vascular dementia, cerebral amyloid angiopathy, dementia with Lewy bodies, chronic traumatic encephalopathy, Down syndrome, or pathological aging.

162. The method of claim 157, wherein the Aβ peptide variant comprises an amino acid sequence having at least 85, 90, 91, 92, 93, 94, 95, 96, 97, 98, or 99% identity with SEQ ID NO:3, or a fragment or functional derivative thereof.

163. The method of claim 157, wherein the Aβ peptide variant comprises an amino acid sequence having at least 85, 90, 91, 92, 93, 94, 95, 96, 97, 98, or 99% identity with SEQ ID NO:4, or a fragment or functional derivative thereof.

164. The method of claim 157, wherein the Aβ peptide variant comprises an amino acid sequence having at least 85, 90, 91, 92, 93, 94, 95, 96, 97, 98, or 99% identity with SEQ ID NO:5, or a fragment or functional derivative thereof.

165. The method of claim 157, wherein the Aβ peptide variant comprises an amino acid sequence having at least 85, 90, 91, 92, 93, 94, 95, 96, 97, 98, or 99% identity with SEQ ID NO:6, or a fragment or functional derivative thereof.

166. The method of claim 157, wherein the vector that encodes the Aβ peptide variant encodes a nucleotide sequence corresponding to an amino acid sequence comprising a truncated beta-carboxyl-terminal fragment (β-CTF) of amyloid precursor protein.

167. The method of claim 157, wherein the truncated β-CTF is fused to a signal peptide sequence, wherein the signal peptide sequence comprises a nucleotide sequence corresponding to an amino acid sequence comprising a Gaussia luciferase signal peptide or a nucleotide sequence corresponding to an amino acid sequence comprising a mouse immunoglobulin heavy chain signal peptide.

168. The method of claim 166, wherein the truncated β-CTF comprises the Aβ peptide variant sequence, a transmembrane domain sequence, and a cytosolic sequence.

169. The method of claim 168, wherein the cytosolic sequence comprises a nucleotide sequence corresponding to an amino acid sequence for membrane anchoring, promotion of gamma-secretase cleavage, and/or extracellular release of Aβ peptide variants.

170. The method of claim 157, wherein the vector comprises an amino acid sequence having at least 85, 90, 91, 92, 93, 94, 95, 96, 97, 98, or 99% identity with the one selected from the group consist of SEQ ID NO:8, SEQ ID NO:35, SEQ ID NO:36, SEQ ID NO:37, SEQ ID NO:38, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, and SEQ ID NO: 80.

171. The method of claim 157, wherein expression of the vector is regulated by a constitutive promoter, a tissue-specific promoter such as a human synapsin I or a choroid plexus specific promoter, or cell-specific promoter, such as prlr, Spint2, or F5.

172. The method of claim 157, wherein the vector is an adenoviral, lentiviral, retroviral, or adeno-associated viral vector.

173. The method of claim 157, wherein the vector is an AAV vector.

174. The method of claim 157, wherein the vector is AAV1, AAV2, AAV3, AAV4, AAV5, AAV6, AAV7, AAV8, AAV9, AAV10, AAV11, AAV12, AAV2.5, AAvDJ, AAVrhlO.XX, AAVrh.8, AAVrh.10, AAVrh.43, AAVpi.2, AAVhu.11, AAVhu.32, AAVhu.37, or PHP.eB AAV.

175. The method of claim 157, wherein the composition further comprises a pharmaceutically acceptable carrier.

176. The method of claim 157, wherein the composition is delivered systemically or locally.
